SEQUENCE LISTING (80 sequences)

SEQ ID NO: 1 - DNA, 52 bp, Artificial Sequence
  Synthetic Polynucleotide
aacaccgtaagtcggagtactgtcctgttttagagctagaaacggtgctttt

SEQ ID NO: 2 - RNA, 100 nt, Artificial Sequence
  Synthetic Polynucleotide
guaagucggaguacuguccuguuuuagagcuagaaauagcaaguuaaaauaaggcuaguccguuaucaacuugaaaaaguggcaccgagucggugcuuuu

SEQ ID NO: 3 - DNA, 20 bp, Artificial Sequence
  Synthetic Polynucleotide
gtaagtcggagtactgtcct

SEQ ID NO: 4 - DNA, 52 bp, Artificial Sequence
  Synthetic Polynucleotide
  misc_feature (27)..(27): n is a, c, g, or t
aacaccgtaagtcggagtactgtcctnggttagagctagaaacggtgctttt

SEQ ID NO: 5 - RNA, 100 nt, Artificial Sequence
  Synthetic Polynucleotide
  misc_feature (21)..(21): n is a, c, g, or u
guaagucggaguacuguccungguuagagcuagaaauagcaaguuaaaauaaggcuaguccguuaucaacuugaaaaaguggcaccgagucggugcuuuu

SEQ ID NO: 6 - DNA, 31 bp, Artificial Sequence
  Synthetic Polynucleotide
gcagagatccagtttggggggttccgcgcac

SEQ ID NO: 7 - DNA, 32 bp, Artificial Sequence
  Synthetic Polynucleotide
cccggtagaattcctcgacgtctaatgccaac

SEQ ID NO: 8 - DNA, 421 bp, Artificial Sequence
  Synthetic Polynucleotide
tgtacaaaaaagcaggctttaaaggaaccaattcagtcgactggatccggtaccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacggaacaccgtaagtcggagtactgtcctgttttagagctagaaatagcaagttaaaataaggctagtccgttatcaacttgaaaaagtggcaccgagtcggtgcttttttt

SEQ ID NO: 9 - DNA, 420 bp, Artificial Sequence
  Synthetic Polynucleotide
tgtacaaaaaagcaggctttaaaggaaccaattcagtcgactggatccggtaccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtaagtcggagtactgtcctgggttagagctagaaatagcaagttaaaataaggctagtccgttatcaacttgaaaaagtggcaccgagtcggtgctttttt

SEQ ID NO: 10 - DNA, 420 bp, Artificial Sequence
  Synthetic Polynucleotide
tgtacaaaaaagcaggctttaaaggaaccaattcagtcgactggatccggtaccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtaagtcggagtactgtcctgggttagagctagaaatagcaagttaacctaaggctagtccgttatcaacttgaaaaagtggcaccgagtcggtgctttttt

SEQ ID NO: 11 - DNA, 420 bp, Artificial Sequence
  Synthetic Polynucleotide
tgtacaaaaaagcaggctttaaaggaaccaattcagtcgactggatccggtaccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtaagtcggagtactgtcctcggttagagctagaaatagcaagttaaccgaaggctagtccgttatcaacttgaaaaagtggcaccgagtcggtgctttttt

SEQ ID NO: 12 - DNA, 422 bp, Artificial Sequence
  Synthetic Polynucleotide
tgtacaaaaaagcaggctttaaaggaaccaattcagtcgactggatccggtaccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtaagtcggagtactgtcctcggttttagagctagaaatagcaagttaaaataaggctagtccgttatcaacttgaaaaagtggcaccgagtcggtgctttttt

SEQ ID NO: 13 - DNA, 422 bp, Artificial Sequence
  Synthetic Polynucleotide
tgtacaaaaaagcaggctttaaaggaaccaattcagtcgactggatccggtaccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtaagtcggagtactgtcctgggttttagagctagaaatagcaagttaaaataaggctagtccgttatcaacttgaaaaagtggcaccgagtcggtgctttttt

SEQ ID NO: 14 - DNA, 5301 bp, Artificial Sequence
  Synthetic Polynucleotide
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttatgaaccgtcagatccgagctcatcaccggtgcgctgccaccatggacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaagcgtcctgctgctactaagaaagctggtcaagctaagaaaaagaaagctagcggcagcggcgccggatccccaaagaagaaaaggaaggttgaagaccccaagaaaaagaggaaggtgatacccgggtaagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagacgccattggggccaatacgcccgcgtttcttccttttccccaccccaccccccaagttcgggtgaaggcccagggctcgcagccaacgtcggggcggcaggccctgccatagcctcag

SEQ ID NO: 15 - DNA, 429 bp, Artificial Sequence
  Synthetic Polynucleotide
tgtacaaaaaagcaggctttaaaggaaccaattcagtcgactggatccggtaccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgcggtctgcgataagtcggagtactgtcctgggttagagctagaaatagcaagttaacctaaggctagtccgttatcaacttgaaaaagtggcaccgagtcggtgcttttt

SEQ ID NO: 16 - DNA, 430 bp, Artificial Sequence
  Synthetic Polynucleotide
tgtacaaaaaagcaggctttaaaggaaccaattcagtcgactggatccggtaccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgcaaatacctcacacactcccaatacatgaagggttagagctagaaatagcaagttaacctaaggctagtccgttatcaacttgaaaaagtggcaccgagtcggtgcttttt

SEQ ID NO: 17 - DNA, 441 bp, Artificial Sequence
  Synthetic Polynucleotide
tgtacaaaaaagcaggctttaaaggaaccaattcagtcgactggatccggtaccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtcaccacattatatcaattacttcttaaatcacacaatcagggttagagctagaaatagcaagttaacctaaggctagtccgttatcaacttgaaaaagtggcaccgagtcggtgctttttt

SEQ ID NO: 18 - DNA, 471 bp, Artificial Sequence
  Synthetic Polynucleotide
tgtacaaaaaagcaggctttaaaggaaccaattcagtcgactggatccggtaccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacggaacaccgcaaatacctcacacactcccaatacatgaatcaccacattatatcaattacttcttaaatcacacaatcagggttagagctagaaatagcaagttaacctaaggctagtccgttatcaacttgaaaaagtggcaccgagtcggtgctttttt

SEQ ID NO: 19 - DNA, 6165 bp, Artificial Sequence
  Synthetic Polynucleotide
gcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcgggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagcttgggctgcaggtcgactctagaggatccccgggtaccggtcgccaacgcgtgccaccatggacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaagcgtcctgctgctactaagaaagctggtcaagctaagaaaaagaaagctagcggcagcggcgccggatccccaaagaagaaaaggaaggttgaagaccccaagaaaaagaggaaggtgataagcgctggaagcggagctactaacttcagcctgctgaagcaggctggagacgtggaggagaaccctggacctaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacatccgcgccccgcaacctccccttctacgagcggctcggcttcaccgtcaccgccgacgtcgaggtgcccgaaggaccgcgcacctggtgcatgacccgcaagcccggtgcctga

SEQ ID NO: 20 - DNA, 2746 bp, Artificial Sequence
  Synthetic Polynucleotide
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctctagaggatccccgggtaccggtcgccaccatgccgaaaagtgccaccttgtacaaaaaagcaggctttaaaggaaccaattcagtcgactggatccggtaccaaggtcgggcaggaagagggcctatttcccatgattccttcatatttgcatatacgatacaaggctgttcgagagataatttgaatttatttgactgtaaacacaaagatattagtacaaaatacgtgacgtcgaaagtaataatttcttgggtagtttgcagttttaaaattatgtttttaaatggactatcatatgcttaccgtaacttgaaagtatttcgatttcttggctttatatatcttgtggaaaggacgaaacaccgattcatctcatctatcagaaacaacagggttggagcaagaaattgcaagtcaacctaaggctagtccgttatcaacttgcaaaagtggcaccgagtcggtgcttttttaccggaagcggagctactcacttcagcctgctgaagcaggctggagacgtggaggagaaccctggacctgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaggccggccagccacggcttcccccctgaggtggccgctcaggacgatggcaccctgcccatgagctgcgcccaggagagcggcatggacaggcaccccgccgcttgcgccagcgctaggatcaacgtgggtgagggcagaggaagtcttctaacatgcggtgacgtggaggagaatccgggccctgtgagcaagggcgaggaggataactccgccatcatcaaggagttcctgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctctctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtga

SEQ ID NO: 21 - DNA, 2745 bp, Artificial Sequence
  Synthetic Polynucleotide
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctctagaggatccccgggtaccggtcgccaccatgccgaaaagtgccaccttgtacaaaaaagcaggctttaaaggaaccaattcagtcgactggatccggtaccaaggtcgggcaggaagagggcctatttcccatgattccttcatatttgcatatacgatacaaggctgttcgagagataatttgaatttatttgactgtaaacacaaagatattagtacaaaatacgtgacgtcgaaagtaataatttcttgggtagtttgcagttttaaaattatgtttttaaatggactatcatatgcttaccgtaacttgaaagtatttcgatttcttggctttatatatcttgtggaaaggacgaaacaccgttcatctcatctatcagaaacaacagggttggagcaagaaattgcaagtcaacctaaggctagtccgttatcaacttgcaaaagtggcaccgagtcggtgcttttttaccggaagcggagctactcacttcagcctgctgaagcaggctggagacgtggaggagaaccctggacctgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaggccggccagccacggcttcccccctgaggtggccgctcaggacgatggcaccctgcccatgagctgcgcccaggagagcggcatggacaggcaccccgccgcttgcgccagcgctaggatcaacgtgggtgagggcagaggaagtcttctaacatgcggtgacgtggaggagaatccgggccctgtgagcaagggcgaggaggataactccgccatcatcaaggagttcctgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctctctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtga

SEQ ID NO: 22 - DNA, 2744 bp, Artificial Sequence
  Synthetic Polynucleotide
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctctagaggatccccgggtaccggtcgccaccatgccgaaaagtgccaccttgtacaaaaaagcaggctttaaaggaaccaattcagtcgactggatccggtaccaaggtcgggcaggaagagggcctatttcccatgattccttcatatttgcatatacgatacaaggctgttcgagagataatttgaatttatttgactgtaaacacaaagatattagtacaaaatacgtgacgtcgaaagtaataatttcttgggtagtttgcagttttaaaattatgtttttaaatggactatcatatgcttaccgtaacttgaaagtatttcgatttcttggctttatatatcttgtggaaaggacgaaacaccgtcatctcatctatcagaaacaacagggttggagcaagaaattgcaagtcaacctaaggctagtccgttatcaacttgcaaaagtggcaccgagtcggtgcttttttaccggaagcggagctactcacttcagcctgctgaagcaggctggagacgtggaggagaaccctggacctgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaggccggccagccacggcttcccccctgaggtggccgctcaggacgatggcaccctgcccatgagctgcgcccaggagagcggcatggacaggcaccccgccgcttgcgccagcgctaggatcaacgtgggtgagggcagaggaagtcttctaacatgcggtgacgtggaggagaatccgggccctgtgagcaagggcgaggaggataactccgccatcatcaaggagttcctgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctctctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtga

SEQ ID NO: 23 - DNA, 2838 bp, Artificial Sequence
  Synthetic Polynucleotide
tgtacaaaaaagcaggctttaaaggaaccaattcagtcgactggatccggtaccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgattcatctcatctatcagaaacaacagttttagagctagaaatagcaagttaaaataaggctagtccgttatcaacttgaaaaagtggcaccgagtcggtgctttttttctagacccagctttcttgtacaaagttggcattagacgtcgaggctagcccagacttaattaatagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctctagaggatccccgggtaccggtcgccaccatgccgaaaagtgccaccgattcatctcatctatcagaaacaacagggccggaagcggagctactcacttcagcctgctgaagcaggctggagacgtggaggagaaccctggacctgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaggccggccagccacggcttcccccctgaggtggccgctcaggacgatggcaccctgcccatgagctgcgcccaggagagcggcatggacaggcaccccgccgcttgcgccagcgctaggatcaacgtgggtgagggcagaggaagtcttctaacatgcggtgacgtggaggagaatccgggccctgtgagcaagggcgaggaggataactccgccatcatcaaggagttcctgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctctctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtga

SEQ ID NO: 24 - DNA, 2837 bp, Artificial Sequence
  Synthetic Polynucleotide
tgtacaaaaaagcaggctttaaaggaaccaattcagtcgactggatccggtaccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgattcatctcatctatcagaaacaacagttttagagctagaaatagcaagttaaaataaggctagtccgttatcaacttgaaaaagtggcaccgagtcggtgctttttttctagacccagctttcttgtacaaagttggcattagacgtcgaggctagcccagacttaattaatagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctctagaggatccccgggtaccggtcgccaccatgccgaaaagtgccaccgttcatctcatctatcagaaacaacagggccggaagcggagctactcacttcagcctgctgaagcaggctggagacgtggaggagaaccctggacctgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaggccggccagccacggcttcccccctgaggtggccgctcaggacgatggcaccctgcccatgagctgcgcccaggagagcggcatggacaggcaccccgccgcttgcgccagcgctaggatcaacgtgggtgagggcagaggaagtcttctaacatgcggtgacgtggaggagaatccgggccctgtgagcaagggcgaggaggataactccgccatcatcaaggagttcctgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctctctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtga

SEQ ID NO: 25 - DNA, 2836 bp, Artificial Sequence
  Synthetic Polynucleotide
tgtacaaaaaagcaggctttaaaggaaccaattcagtcgactggatccggtaccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgattcatctcatctatcagaaacaacagttttagagctagaaatagcaagttaaaataaggctagtccgttatcaacttgaaaaagtggcaccgagtcggtgctttttttctagacccagctttcttgtacaaagttggcattagacgtcgaggctagcccagacttaattaatagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctctagaggatccccgggtaccggtcgccaccatgccgaaaagtgccaccgtcatctcatctatcagaaacaacagggccggaagcggagctactcacttcagcctgctgaagcaggctggagacgtggaggagaaccctggacctgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaggccggccagccacggcttcccccctgaggtggccgctcaggacgatggcaccctgcccatgagctgcgcccaggagagcggcatggacaggcaccccgccgcttgcgccagcgctaggatcaacgtgggtgagggcagaggaagtcttctaacatgcggtgacgtggaggagaatccgggccctgtgagcaagggcgaggaggataactccgccatcatcaaggagttcctgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctctctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtga

SEQ ID NO: 26 - DNA, 450 bp, Artificial Sequence
  Synthetic Polynucleotide
  misc_feature (429)..(444): n is a, c, g, or t
tgtacaaaaaagcaggctttaaaggaaccaattcagtcgactggatccggtaccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtaagtcggagtactgtcctgggttagagctagaaatagcaagttaacctaaggctagtccgttatcaacttgaaaaagtggcaccgagtcggtgcttttttgcaagcagnnnnnnnnnnnnnnnntctaga

SEQ ID NO: 27 - DNA, 450 bp, Artificial Sequence
  Synthetic Polynucleotide
  misc_feature (429)..(444): n is a, c, g, or t
tgtacaaaaaagcaggctttaaaggaaccaattcagtcgactggatccggtaccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggtggctttaccaacagtacgggttagagctagaaatagcaagttaacctaaggctagtccgttatcaacttgaaaaagtggcaccgagtcggtgcttttttgcaagcagnnnnnnnnnnnnnnnntctaga

SEQ ID NO: 28 - DNA, 461 bp, Artificial Sequence
  Synthetic Polynucleotide
  misc_feature (440)..(455): n is a, c, g, or t
tgtacaaaaaagcaggctttaaaggaaccaattcagtcgactggatccggtaccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgattcatctcatctatcagaaaataaataaagggttagagctagaaatagcaagttaacctaaggctagtccgttatcaacttgaaaaagtggcaccgagtcggtgcttttttgcaagcagnnnnnnnnnnnnnnnntctaga

SEQ ID NO: 29 - DNA, 461 bp, Artificial Sequence
  Synthetic Polynucleotide
  misc_feature (440)..(455): n is a, c, g, or t
tgtacaaaaaagcaggctttaaaggaaccaattcagtcgactggatccggtaccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgcaaatacctcacacactcccaatacatgaagggttagagctagaaatagcaagttaacctaaggctagtccgttatcaacttgaaaaagtggcaccgagtcggtgcttttttgcaagcagnnnnnnnnnnnnnnnntctaga

SEQ ID NO: 30 - DNA, 471 bp, Artificial Sequence
  Synthetic Polynucleotide
  misc_feature (450)..(465): n is a, c, g, or t
tgtacaaaaaagcaggctttaaaggaaccaattcagtcgactggatccggtaccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtcaccacattatatcaattacttcttaaatcacacaatcagggttagagctagaaatagcaagttaacctaaggctagtccgttatcaacttgaaaaagtggcaccgagtcggtgcttttttgcaagcagnnnnnnnnnnnnnnnntctaga

SEQ ID NO: 31 - DNA, 471 bp, Artificial Sequence
  Synthetic Polynucleotide
  misc_feature (450)..(465): n is a, c, g, or t
tgtacaaaaaagcaggctttaaaggaaccaattcagtcgactggatccggtaccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgttacaaaatacaattaattaaaactacatcaaaacacacagggttagagctagaaatagcaagttaacctaaggctagtccgttatcaacttgaaaaagtggcaccgagtcggtgcttttttgcaagcagnnnnnnnnnnnnnnnntctaga

SEQ ID NO: 32 - DNA, 662 bp, Artificial Sequence
  Synthetic Polynucleotide
tgtacaaaaaagcaggctttaaaggaaccaattcagtcgactggatccggtaccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtaagtcggagtactgtcctgttttagagctagaaatagcaagttaaaataaggctagtccgttatcaacttgaaaaagtggcaccgagtcggtgctttttttctagaatcgctaaactgcgtcgcggagccttatggcataggtcgtccgcggagcattccggtaacgcttatggtccatagcacattcatcgcatccgggcgtgcgctctatttgacgatcccttggcgcagaggtgctggccacgtgctaaattaaagcggctgcactactgtaaggtccgtcggccgtcgatccaccgattcgcgtcgtgcgtaagtcggagtactgtcctggggctagc

SEQ ID NO: 33 - DNA, 681 bp, Artificial Sequence
  Synthetic Polynucleotide
tgtacaaaaaagcaggctttaaaggaaccaattcagtcgactggatccggtaccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgcaaatacctcacacactcccaatacatgaagttttagagctagaaatagcaagttaaaataaggctagtccgttatcaacttgaaaaagtggcaccgagtcggtgctttttttctagaatcgctaaactgcgtcgcggagccttatggcatagtcgtccgcggagcattccggtaacgcttatggtccatagcacattcatcgcatccgggcgtgcgctctatttgacgatcccttggcgcagagggctggccagtgctaaattaaagcggctgcactactgtaaggtccgtcggccgtcgatccaccgattcgcgtcgtgcgcaaatacctcacacactcccaatacatgaaggggctagc

SEQ ID NO: 34 - DNA, 703 bp, Artificial Sequence
  Synthetic Polynucleotide
tgtacaaaaaagcaggctttaaaggaaccaattcagtcgactggatccggtaccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtcaccacattatatcaattacttcttaaatcacacaatcagttttagagctagaaatagcaagttaaaataaggctagtccgttatcaacttgaaaaagtggcaccgagtcggtgctttttttctagaatcgctaaactgcgtcgcggagccttatggcatagtcgtccgcggagcattccggtaacgcttatggtccatagcacattcatcgcatccgggcgtgcgctctatttgacgatcccttggcgcagaggtgctggccacgtgctaaattaaagcggctgcactactgtaaggtccgtcggccgtcgatccaccgattcgcgtcgtgcgtcaccacattatatcaattacttcttaaatcacacaatcaggggctagc

SEQ ID NO: 35 - DNA, 3474 bp, Artificial Sequence
  Synthetic Polynucleotide
gcgccgggttttggcgcctcccgcgggcgcccccctcctcacggcgagcgctgccacgtcagacgaagggcgcaggagcgttcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagttgcgggctgctgggctggccggggctttcgtggccgccgggccgctcggtgggacggaagcgtgtggagagaccgccaagggctgtagtctgggtccgcgagcaaggttgccctgaactgggggttggggggagcgcacaaaatggcggctgttcccgagtcttgaatggaagacgcttgtaaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcgggtttgtcgtctggttgcgggggcggcagttatgcggtgccgttgggcagtgcacccgtacctttgggagcgcgcgcctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacaggatccccgggtaccggtcgccaccatgtctcggttggacaaatctaaagtaatcaactctgcactggaattgctgaacgaggtaggcatagagggcctcacaacgaggaagctggcccaaaagctgggcgtcgaacagccaaccctgtactggcacgtcaagaataaaagggctctcctggacgcgctggcaattgagatgctcgacagacaccatacacacttttgcccccttgaaggggaatcctggcaggacttcctgcgaaacaatgccaagtcatttagatgcgctcttctgtctcatcgggacggtgctaaggtgcatctgggtacaagacccacggaaaagcagtatgagacactggaaaatcaactggcctttttgtgtcagcagggcttctctctcgaaaacgcgctttacgcgctgtcagccgtgggtcattttaccctgggctgcgtgctggaggaccaggagcatcaagtggctaaggaggaacgggaaacccctaccaccgactctatgccacctctcttgcggcaggcaattgagttgttcgaccaccagggtgccgagccggccttcctgttcggcttggagcttatcatctgcggcctggagaagcagctgaagtgtgagagtggaagtcgtacgggaagcggagctactaacttcagcctgctgaagcaggctggagacgtggaggagaaccctggacctaaaccagtaacattgtatgatgtcgcagagtatgccggtgtctcttatcagactgtttccagagtggtgaaccaggccagccatgtttctgccaaaaccagggaaaaagtggaagcagccatggcagagctgaattacattcccaacagagtggcacaacaactggcaggcaaacagagcttgctgattggagttgccacctccagtctggccctgcatgcaccatctcaaattgtggcagccattaaatctagagctgatcaactgggagcctctgtggtggtgtcaatggtagaaagaagtggagttgaagcctgtaaagctgctgtgcacaatcttctggcacaaagagtcagtgggctgatcattaactatccactggatgaccaggatgccattgctgtggaagctgcctgcactaatgttccagcactctttcttgatgtctctgaccagacacccatcaacagtattattttctcccatgaagatggtacaagactgggtgtggagcatctggttgcattgggacaccagcaaattgcactgcttgcgggcccactcagttctgtctcagcaaggctgagactggctggctggcataaatatctcactaggaatcaaattcagccaatagctgaaagagaaggggactggagtgccatgtctgggtttcaacaaaccatgcaaatgctgaatgagggcattgttcccactgcaatgctggttgccaatgatcagatggcactgggtgcaatgagagccattactgagtctgggctgagagttggtgcagatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgccagtctcactggtgaagagaaaaaccaccctggcacccaatacacaaactgcctctccccgggcattggctgattcactcatgcagctagcaagacaggtttccagactggaaagtgggcagagcagcctgaggcctcctaagaagaagaggaaggttggctctggtgcaaccaatttctctcttcttaaacaagccggtgatgtggaggagaaccccggacccgccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactga

SEQ ID NO: 36 - DNA, 338 bp, Artificial Sequence
  Synthetic Polynucleotide
gaatcctatgcttcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagtccctatcagtgatagagatcccaagtcgcgtgtagcgaagcagggttagagctagaaatagcaagttaacctaaggctagtccgttatcaacttgaaaaagtggcaccgagtcggtgctttttt

SEQ ID NO: 37 - DNA, 452 bp, Artificial Sequence
  Synthetic Polynucleotide
tgtacaaaaaagcaggctttaaaggaaccaattcagtcgactggatccggtaccaaggtcgggcaggaagagggcctatttcccatgattccttcatatttgcatatacgatacaaggctgttagagagataattagaattaatttgactgtaaacacaaagatattagtacaaaaaattgtgagcggataacaattatttcttgggtagtttgcagttttaaaattatgttttaaaatggactatcatatgcttaccgtaacttgaaagtaattgtgagcgctcacaattatatatcttgtggaaaggacgaaacaccgagtcgcgtgtagcgaagcagggttagagctagaaatagcaagttaacctaaggctagtccgttatcaacttgaaaaagtggcaccgagtcggtgcttttttctagacccagcaattgtgagcgctcacaatt

SEQ ID NO: 38 - DNA, 826 bp, Artificial Sequence
  Synthetic Polynucleotide
gaatcctatgcttcgaacgctcacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagtccctatcagtgatagagatcccagtggctttaccaacagtacgggttagagctagaaatagcaagttaacctaaggctagtccgttatcaacttgaaaaagtggcaccgagtcggtgctttttttcacgaggcggacactgattgacacggtttgctagctgtacaaaaaagcaggctttaaaggaaccaattcagtcgactggatccggtaccaaggtcgggcaggaagagggcctatttcccatgattccttcatatttgcatatacgatacaaggctgttagagagataattagaattaatttgactgtaaacacaaagatattagtacaaaaaattgtgagcggataacaattatttcttgggtagtttgcagttttaaaattatgttttaaaatggactatcatatgcttaccgtaacttgaaagtaattgtgagcgctcacaattatatatcttgtggaaaggacgaaacaccgagtcgcgtgtagcgaagcagggttagagctagaaatagcaagttaacctaaggctagtccgttatcaacttgaaaaagtggcaccgagtcggtgcttttttctagacccagcaattgtgagcgctcacaatt

SEQ ID NO: 39 - DNA, 1649 bp, Artificial Sequence
  Synthetic Polynucleotide
ggggactttccgggaatttccggggactttccgggaatttccgggaatttccggggactttccgggaatttccggggactttccgggaatttccagatctggcctcggcggccaagcttgctagcggggggctataaaagggggtgggggcgttcgtcctcactctagttctgcgatctaagtaagcttggcattaccggtcgccaacgcgtgccaccatggtgagcgagctgattaaggagaacatgcacatgaagctgtacatggagggcaccgtgaacaaccaccacttcaagtgcacatccgagggcgaaggcaagccctacgagggcacccagaccatgagaatcaaggcggtcgagggcggccctctccccttcgccttcgacatcctggctaccagcttcatgtacggcagcaaaaccttcatcaaccacacccagggcatccccgacttctttaagcagtccttccccgagggcttcacatgggagagagtcaccacatacgaagatgggggcgtgctgaccgctacccaggacaccagcctccaggacggctgcctcatctacaacgtcaagatcagaggggtgaacttcccatccaacggccctgtgatgcagaagaaaacactcggctgggaggcctccaccgagacactgtaccccgctgacggcggcctggaaggcagagccgacatggccctgaagctcgtgggcgggggccacctgatctgcaaccttaagaccacatacagatccaagaaacccgctaagaacctcaagatgcccggcgtctactatgtggacaggagactggaaagaatcaaggaggccgacaaagagacatacgtcgagcagcacgaggtggctgtggccagatactgcgacctccctagcaaactggggcacaaacttaattccggatccccaaagaagaaaaggaaggttgaagaccccaagaaaaagaggaaggtgataagcgctggaagcggagctactaacttcagcctgctgaagcaggctggagacgtggaggagaaccctggacctaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacatccgcgccccgcaacctccccttctacgagcggctcggcttcaccgtcaccgccgacgtcgaggtgcccgaaggaccgcgcacctggtgcatgacccgcaagcccggtgcctga

SEQ ID NO: 40 - DNA, 5114 bp, Artificial Sequence
  Synthetic Polynucleotide
ggggactttccgggaatttccggggactttccgggaatttccgggaatttccggggactttccgggaatttccggggactttccgggaatttccagatctggcctcggcggccaagcttgctagcggggggctataaaagggggtgggggcgttcgtcctcactctagttctgcgatctaagtaagcttggcattaccggtcgccaacgcgtgccaccatggacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaagcgtcctgctgctactaagaaagctggtcaagctaagaaaaagaaagctagcggcagcggcgccggatccccaaagaagaaaaggaaggttgaagaccccaagaaaaagaggaaggtgataagcgctggaagcggagctactaacttcagcctgctgaagcaggctggagacgtggaggagaaccctggacctaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacatccgcgccccgcaacctccccttctacgagcggctcggcttcaccgtcaccgccgacgtcgaggtgcccgaaggaccgcgcacctggtgcatgacccgcaagcccggtgcctga

SEQ ID NO: 41 - DNA, 29 bp, Artificial Sequence
  Synthetic Polynucleotide
aatgatacggcgaccaccgagatctacac

SEQ ID NO: 42 - DNA, 24 bp, Artificial Sequence
  Synthetic Polynucleotide
caagcagaagacggcatacgagat

SEQ ID NO: 43 - DNA, 52 bp, Artificial Sequence
  Synthetic Polynucleotide
aaaagcaccgtttctagctctaaaacaggacagtactccgacttacggtgtt

SEQ ID NO: 44 - DNA, 52 bp, Artificial Sequence
  Synthetic Polynucleotide
  misc_feature (26)..(26): n is a, c, g, or t
aaaagcaccgtttctagctctaaccnaggacagtactccgacttacggtgtt

SEQ ID NO: 45 - RNA, 99 nt, Artificial Sequence
  Synthetic Polynucleotide
uaagucggaguacuguccuguuuuagagcuagaaauagcaaguuaaaauaaggcuaguccguuaucaacuugaaaaaguggcaccgagucggugcuuuu

SEQ ID NO: 46 - RNA, 99 nt, Artificial Sequence
  Synthetic Polynucleotide
uaagucggaguacuguccuggguuagagcuagaaauagcaaguuaaccuaaggcuaguccguuaucaacuugaaaaaguggcaccgagucggugcuuuu

SEQ ID NO: 47 - DNA, 57 bp, Artificial Sequence
  Synthetic Polynucleotide
ggaaaggacgaaacaccgtaagtcggagtactgtcctgggttagagctagaaatagc

SEQ ID NO: 48 - DNA, 56 bp, Artificial Sequence
  Synthetic Polynucleotide
ggaaaggacgaaacaccgtaagtcggagtactgcctgggttagagctagaaatagc

SEQ ID NO: 49 - DNA, 52 bp, Artificial Sequence
  Synthetic Polynucleotide
ggaaaggacgaaacaccgtaagtcggagtactgggttagagctagaaatagc

SEQ ID NO: 50 - DNA, 50 bp, Artificial Sequence
  Synthetic Polynucleotide
ggaaaggacgaaacaccgtaagtcggagtactgttagagctagaaatagc

SEQ ID NO: 51 - DNA, 39 bp, Artificial Sequence
  Synthetic Polynucleotide
ggaaaggacgaaacaccgtgggttagagctagaaatagc

SEQ ID NO: 52 - DNA, 23 bp, Artificial Sequence
  Synthetic Polynucleotide
taagtcggagtactgctcctggg

SEQ ID NO: 53 - DNA, 47 bp, Artificial Sequence
  Synthetic Polynucleotide
ttgacttgcaatttcttgctccaaccctgatgagatgaatcggtgtt

SEQ ID NO: 54 - DNA, 41 bp, Artificial Sequence
  Synthetic Polynucleotide
ttgacttgcaatttcttgctccgatgagatgaatcgatgtt

SEQ ID NO: 55 - DNA, 59 bp, Artificial Sequence
  Synthetic Polynucleotide
ttgacttgcaatttcttgctccaaccctgtgtttctgatagatgagatgaatcggtgtt

SEQ ID NO: 56 - DNA, 37 bp, Artificial Sequence
  Synthetic Polynucleotide
ttgacttgcaatttcttgctccaaccctgttggtgtt

SEQ ID NO: 57 - DNA, 61 bp, Artificial Sequence
  Synthetic Polynucleotide
ttgacttgcaatttcttgctccaaccctgtttgtttctgatagatgagatgaatcggtgtt

SEQ ID NO: 58 - DNA, 49 bp, Artificial Sequence
  Synthetic Polynucleotide
ttgacttgcaatttcttgctccatctgatagatgagatgaatcggtgtt

SEQ ID NO: 59 - DNA, 32 bp, Artificial Sequence
  Synthetic Polynucleotide
ttgacttgcaatagatgagatgaatcggtgtt

SEQ ID NO: 60 - DNA, 50 bp, Artificial Sequence
  Synthetic Polynucleotide
ttgacttgcaatttcttgctccaaccctgttgatgagatgaatcgatgtt

SEQ ID NO: 61 - DNA, 41 bp, Artificial Sequence
  Synthetic Polynucleotide
ttgacttgcaatttcttgctccaatgagatgaatcggtgtt

SEQ ID NO: 62 - DNA, 46 bp, Artificial Sequence
  Synthetic Polynucleotide
ttgacttgcaatttcttgctccaaccctgtgagatgaatcggtgtt

SEQ ID NO: 63 - DNA, 46 bp, Artificial Sequence
  Synthetic Polynucleotide
ttgacttgcaatttcttgtttctgatagatgagatgaatcggtgtt

SEQ ID NO: 64 - DNA, 52 bp, Artificial Sequence
  Synthetic Polynucleotide
ttgacttgcaatttcttgctccaaccctgtttagatgagatgaatcggtgtt

SEQ ID NO: 65 - DNA, 44 bp, Artificial Sequence
  Synthetic Polynucleotide
ttgacttgcaatttcttgctccaaccctgagatgaatcggtgtt

SEQ ID NO: 66 - DNA, 56 bp, Artificial Sequence
  Synthetic Polynucleotide
ttgacttgcaatttcttgctccaaccctgttgtttctgatagaacgaatcggtgtt

SEQ ID NO: 67 - DNA, 32 bp, Artificial Sequence
  Synthetic Polynucleotide
  misc_feature (20)..(20): n is a, c, g, or t
ttgacttgcaatttctgggncgaatcggggtt

SEQ ID NO: 68 - DNA, 52 bp, Artificial Sequence
  Synthetic Polynucleotide
ttgacttgcaatttcttgctcctgtttctgatagatgagatgaatcggtgtt

SEQ ID NO: 69 - DNA, 58 bp, Artificial Sequence
  Synthetic Polynucleotide
ttgacttgcaatttcttgctccaaccctgttttctgatagatgagatgaatcggtgtt

SEQ ID NO: 70 - DNA, 46 bp, Artificial Sequence
  Synthetic Polynucleotide
ttgacttgcaatttcttgtttctgatagatgacatgaatcggtgtt

SEQ ID NO: 71 - DNA, 47 bp, Artificial Sequence
  Synthetic Polynucleotide
  misc_feature (23)..(23): n is a, c, g, or t
  misc_feature (31)..(31): n is a, c, g, or t
ttgacttgcaatttcttgctccngccctgangagatgaatcggtgtt

SEQ ID NO: 72 - DNA, 46 bp, Artificial Sequence
  Synthetic Polynucleotide
ttgacttgcaatttcttgctccaaccctgtgagatgaatcggtgtg

SEQ ID NO: 73 - DNA, 56 bp, Artificial Sequence
  Synthetic Polynucleotide
ttgacttgcaatttcttgctccaaccaatttcttgctccaacaacgaatcggtgtt

SEQ ID NO: 74 - DNA, 62 bp, Artificial Sequence
  Synthetic Polynucleotide
ttgacttgcaatttcttgctccaaccctgttttgtttctgatagatgagatgaatcggtgtt

SEQ ID NO: 75 - DNA, 56 bp, Artificial Sequence
  Synthetic Polynucleotide
ttgacttgcaatttcttgctccaaccctgttctgatagatgaaatgaatcggtgtt

SEQ ID NO: 76 - DNA, 56 bp, Artificial Sequence
  Synthetic Polynucleotide
ttgacttgcaatttcttgctccaaccctgttctgataaatgagaggaatcggtgtt

SEQ ID NO: 77 - DNA, 36 bp, Artificial Sequence
  Synthetic Polynucleotide
ttgacttgcaatttcttgctccaatgcagcggggtt

SEQ ID NO: 78 - DNA, 68 bp, Artificial Sequence
  Synthetic Polynucleotide
ttgacttgcaatttcttgctccaacaacatctatctattgtttctgatagatgagatgaatcggtgtt

SEQ ID NO: 79 - DNA, 16 bp, Artificial Sequence
  Synthetic Polynucleotide
gacattccaataaaaa

SEQ ID NO: 80 - DNA, 16 bp, Artificial Sequence
  Synthetic Polynucleotide
taaacaccacaaaaaa

(19) United States
(12) Patent Application Publication
(10) Pub. No.: US 2018/0291372 A1
(43) Pub. Date: Oct. 11, 2018

(54) SELF-TARGETING GENOME EDITING SYSTEM

(71) Applicant: Massachusetts Institute of Technology, Cambridge, MA (US)

(72) Inventors: Timothy Kuan-Ta Lu, Cambridge, MA (US); Samuel David Perli, Cambridge, MA (US); Hao Cui, Boston, MA (US)

(73) Assignee: Massachusetts Institute of Technology, Cambridge, MA (US)

(21) Appl. No.: 15/573,879

(22) PCT Filed: May 13, 2016
(86) PCT No.: PCT/US16/32348

§ 371 Date: Nov. 14, 2017

Related U.S. Application Data
(60) Provisional application No. 62/161,766, filed on May 14, 2015.

(51) Int. Cl. C12N 15/11; A61K 35/22; C12N 9/22; C12N 15/86

(52) U.S. Cl. CPC C12N 15/11; A61K 35/22; C12N 9/22; C12N 2740/15043; C12N 2310/20; C12N 2800/80; C12N 15/86

(57) ABSTRACT

The present disclosure is directed, in some embodiments, to engineered nucleic acids comprising a promoter operably linked to a nucleotide sequence encoding a guide ribonucleic acid (gRNA) that comprises a specificity determining sequence (SDS) and a protospacer adjacent motif (PAM). The present disclosure is directed, in some embodiments, to cells comprising, vectors comprising, and methods of producing the engineered nucleic acids.

### RELATED APPLICATIONS

This application claims the benefit under 35 U.S.C. § 119(e) of U.S. provisional application No. 62/161,766, filed May 14, 2015, which is incorporated by reference herein in its entirety.

### FIELD OF THE INVENTION

Aspects of the present disclosure relate to the general field of biotechnology and, more particularly, to engineered nucleic acid technology.

### BACKGROUND OF THE INVENTION

Clustered Regularly Interspaced Short Palindromic Repeats (CRISPR) systems for editing, regulating and targeting genomes comprise at least two distinct components: (1) a guide RNA (gRNA) and (2) the CRISPR-associated (Cas) nuclease, Cas9 (an endonuclease). A gRNA is a single chimeric transcript that combines the targeting specificity of endogenous bacterial CRISPR targeting RNA (crRNA) with the scaffolding properties of trans-activating crRNA (tracrRNA). Typically, a gRNA used for genome editing is transcribed from either a plasmid or a genomic locus within a cell (FIG. 1). The gRNA transcript forms a complex with Cas9, and then the gRNA/Cas9 complex is recruited to a target sequence as a result of the base-pairing between the crRNA sequence and its complementary target sequence in genomic DNA, for example.

### SUMMARY OF THE INVENTION

In a typical synthetic CRISPR/Cas9 genome editing system, a genomic target sequence is modified by designing a gRNA complementary to that sequence of interest, which then directs the gRNA/Cas9 complex to the target (Sander J D et al., Nature Biotechnology 32, 247-355, 2014, incorporated by reference herein). The Cas9 endonuclease “cuts” the genomic target DNA upstream of a protospacer adjacent motif (PAM), resulting in double-strand breaks. Repair of the double-strand breaks often results in inserts or deletions (collectively referred to as “indels”) at the double-strand break site. This CRISPR/Cas9 system is often used to “edit” the genome of a cell, each iteration requiring the design and introduction of a new gRNA sequence specific to a target sequence of interest.

Provided herein is a “self-targeting” (e.g., iterative self-targeting) genome editing platform whereby a gRNA transcribed from a deoxyribonucleic acid (DNA) template (e.g., an episomal vector) within a cell and designed to target, for example, a genomic sequence of interest forms a complex with Cas9, and then guides the complex to the DNA template from which the gRNA was transcribed. Once recruited, Cas9 modifies the DNA template, introducing, for example, an insertion or a deletion. A subsequent round of transcription produces another gRNA having a sequence different from the sequence of the gRNA initially transcribed from the DNA template. This “self-targeting,” in some embodiments, continues in an iterative manner, generating gRNAs, each targeting the nucleic acid from which it was transcribed (and, in some embodiments, targeting a genomic sequence), permitting, for example, a form of “continuous evolution.”

The present disclosure is based, at least in part, on unexpected results showing that introduction of a PAM sequence into DNA encoding gRNA results in gRNA/Cas9 targeting of the DNA, and following Cas9 cleavage of the DNA, the PAM sequence is often preserved, allowing for subsequent rounds of Cas9 cleavage.

Thus, some aspects of the present disclosure provide engineered nucleic acids comprising a promoter operably linked to a nucleotide sequence encoding a gRNA that comprises a specificity determining sequence (SDS) and a protospacer adjacent motif (PAM).

In some embodiments, the PAM is a wild-type PAM. In some embodiments, the PAM is downstream (3′) from the SDS. In some embodiments, the PAM is adjacent to the SDS.

In some embodiments, the nucleotide sequence of the PAM is selected from the group consisting of NGG, NNGRR(T/N), NNNNGATT, NNAGAAW and NAAAAC.

In some embodiments, the length of the SDS is 15 to 30 nucleotides. In some embodiments, the length of the SDS is 20 nucleotides.

In some embodiments, the promoter is inducible.

Some aspects of the present disclosure are directed to cells comprising an (e.g., at least one) engineered nucleic acid as described herein. In some embodiments, the cells comprise at least two engineered nucleic acids.

In some embodiments, the engineered nucleic acid is located in the genome of the cell.

Some aspects of the present disclosure are directed to episomal vectors comprising an (e.g., at least one) engineered nucleic acid as described herein. In some embodiments, an episomal vector is a lentiviral vector.

Some aspects of the present disclosure are directed to cells comprising an (e.g., at least one) episomal vector as described herein.

Some aspects of the present disclosure are directed to methods that comprise introducing into a cell an (e.g., at least one) engineered nucleic acid as described herein. In some embodiments, at least two engineered nucleic acids are introduced into a cell.

Some aspects of the present disclosure are directed to methods that comprise introducing into a cell an (e.g., at least one) episomal vector as described herein. In some embodiments, at least two episomal vectors are introduced into a cell.

Also provided herein are a self-contained analog memory device, comprising an engineered nucleic acid comprising an inducible promoter operably linked to a nucleotide sequence encoding a guide ribonucleic acid (gRNA) that comprises a specificity determining sequence (SDS) and a protospacer adjacent motif (PAM).

In some embodiments, the inducible promoter is regulated by a cell signaling protein. In some embodiments, the cell signaling protein is a cytokine (e.g., a tumor necrosis factor or an interleukin).

Also provided herein are cells comprising the foregoing device and Cas9 nuclease. The cell may be, in some embodiments, a mammalian cell, such as a human cell.

In some embodiments, the Cas9 is a catalytically inactive dCas9.

In some embodiments, the Cas9 (e.g., dCas9) is fused to a DNA modifying protein or protein domain. Proteins with DNA-modifying enzymatic activity are known. Such enzymatic activity may nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity or glycosylase activity. Examples of proteins having DNA modifying domains include, but are not limited to, transferases (e.g., terminal deoxynucleotidyl transferase), RNases (e.g., RNase A, ribonuclease H), DNases (e.g., DNase I), ligases (e.g., T4 DNA ligase, E. coli DNA ligase), nucleases (e.g., 51 nuclease), kinases (e.g., T4 polynucleotide kinase), phoshatases (e.g., calf intestinal alkaline phosphatase, bacterial alkaline phosphatase), exonucleases (e.g., X exonuclease), endonucleases, glycosylases (e.g., uracil DNA glycosylases), deaminases and the like. A variety of proteins having one or more DNA modifying domains are commercially available (e.g., New England Biolabs, Beverly, Mass.; Invitrogen, Carlsbad, Calif.; Sigma-Aldrich, St. Louis, Mo.).

In some embodiments, Cas9 (e.g., dCas9) is fused to a DNA-modifying nuclease, such as FokI nuclease, WT Cas9, ZNF, or nickase. In some embodiments, Cas9 (e.g., dCas9) is fused to a DNA-modifying deaminase, such as cytidine deaminase (e.g., APOBEC1, APOBEC3, APOBEC2, AID) or adenosine deaminase. In some embodiments, Cas9 (e.g., dCas9) is fused to a DNA-modifying epigenetic modifier, such as methyltransferase, acetyltransferase, kinases, phosphorylases, methylase, acetylase or glycosylase.

The present disclosure also provides methods comprising maintaining a cell comprising a self-contained analog memory device under conditions that result in recording of molecular stimuli (e.g., cell signaling protein or other stimuli that regulates an inducible promoter of interest) in the form of DNA mutations in the cell.

Also provided herein are methods comprising delivering the cell to a subject (e.g., a human subject). In some embodiments, the subject has an inflammatory condition (e.g., ankylosing spondylitis, antiphospholipid antibody syndrome, gout, inflammatory arthritis, myositis, rheumatoid arthritis, schleroderma, Sjorgen's syndrome, systemic lupus, erythematosus, inflammatory bowel disease, Crohn's disease, multiple sclerosis, and vasculitis).

The invention is not limited in its application to the details of construction and the arrangement of components set forth in the following description or illustrated in the drawings. The invention is capable of other embodiments and of being practiced or of being carried out in various ways. Each of the above embodiments and aspects may be linked to any other embodiment or aspect. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

### BRIEF DESCRIPTION OF THE DRAWINGS

The accompanying drawings are not intended to be drawn to scale. For purposes of clarity, not every component may be labeled in every drawing.

FIG. 1 depicts a conventional CRISPR/Cas system. A wild-type gRNA is transcribed, which associates with Cas9 to form a Cas9-gRNA complex. The gRNA has perfect homology in the specificity determining sequence (SDS, highlighted in pink) to a target DNA locus in the host genome. Once a double-strand break is introduced in the target DNA by the Cas9-gRNA complex, indels (insertion/deletions) or point mutations are introduced by the non-homologous end joining (NHEJ) error-prone DNA repair pathway on the target DNA.

FIG. 2 depicts one embodiment of a self-targeting genome editing system of the present disclosure. A self-targeting guide RNA (stgRNA) is first transcribed and then associates with Cas9 to form a Cas9-stgRNA complex. The Cas9-stgRNA complex targets the DNA from which the stgRNA was originally transcribed. This is followed by NHEJ-mediated error prone DNA repair. After the error-prone repair, a new, mutated version of the original stgRNA is transcribed, which can once again target the modified DNA from which the mutated version the stgRNA is transcribed. Multiple rounds of transcription and DNA cleavage can occur, resulting in a self-evolving CRISPR-Cas system. The mutated self-targeting gRNAs (stgRNAs) are illustrated to contain white dots (representing mutations) on a dark grey line (representing the original SDS). Over time, mutations in the DNA encoding stgRNAs accumulate, providing a molecular record of the self-evolving action.

FIG. 3A depicts transcription of gRNA in mammalian cells. Immediately following the U6 promoter is the SDS of the gRNA (e.g., GTAAGTCGGAGTACTGTCCT; SEQ ID NO:3). Several RNA secondary structural features of the gRNA are illustrated, including the lower stem, which immediately follows the SDS. FIG. 3B depicts an example of transcription of a self-targeting gRNA (stgRNA), engineered by introducing a 5′-NGG-3′ PAM domain immediately downstream of the SDS. Similar to the wild-type gRNA, the stgRNA was transcribed from the U6 promoter. Introduction of the 5′-NGG-3′ PAM domain resulted in the modification of the gRNA nucleotides U23 and U24 to G23 and G24, respectively. The black arrow indicates the de-stabilization of the RNA secondary structure in the lower stem of the stgRNA resulting from the introduction of the PAM domain.

FIG. 4 depicts an example of an experimental design for assaying self-targeting activity of stgRNAs.

FIG. 5 depicts an example of a gRNA sequence modified to contain a PAM motif, which enables self-targeted cleavage via Cas9.

FIG. 6 depicts results from an experiment showing that in addition to U23→G23 and U24→G24 mutations, compensatory A49→C49 and A48→C48 mutations mediate self-targeting activity.

FIG. 7 depicts results from an experiment showing that additional Cas9 mutants did not improve self-targeting efficiency.

FIG. 8 depicts sample modified sequences from self-targeting activity.

FIG. 9 depicts the experimental design for a time course analysis of stgRNA evolution.

FIG. 10 depicts a time course characterization of control, wild-type gRNA sequences.

FIG. 11 depicts a time course characterization of stgRNA sequences.

FIG. 12 depicts a time course characterization of insertions per base position in DNA encoding a stgRNA.

FIG. 13 depicts a time course characterization of deletions per base position in the DNA encoding the stgRNA.

FIG. 14 depicts results obtained from T7 E1A assays for stable cell lines expressing stgRNAs with 20 nucleotide (nt) SDS or 70 nt SDS.

FIG. 15 depicts computationally designed 30, 40 and 70 nt SDS containing stgRNAs demonstrate self-targeted cleavage activity.

FIGS. 16A-16D depict Dox and TNFα inducible self-evolving CRISPR/Cas. FIGS. 16A and 16B are schematics illustrating the genetic constructs used for building Doxycycline (Dox) and Tumor Necrosis Factor-alpha (TNFα) Cas9 cell lines. FIG. 16C and FIG. 16D show a gel image of polymerase chain reaction (PCR)-amplified genomic DNA (see Example 11).

FIG. 17A-17E depict examples of continuously evolving self-targeting guide RNAs. FIG. 17A is a schematic of a self-targeting CRISPR-Cas system. The Cas9-stgRNA complex cleaves the DNA from which the stgRNA is transcribed, leading to error-prone DNA repair. Multiple rounds of transcription and DNA cleavage can occur, resulting in continuous mutagenesis of the DNA encoding the stgRNA. The light gray line in the stgRNA schematic represents the specificity-determining sequence (SDS) while mutations in the stgRNAs are illustrated as dark gray marks. When stgRNA or Cas9 expression is linked to cellular events of interest, accumulation of mutations at the stgRNA locus provides a molecular record of those cellular events. FIG. 17B shows multiple variants of sgRNAs that were built and tested for inducing mutations at their own encoding locus using a T7 endonuclease I DNA mutation detection assay. Introducing a PAM into the DNA encoding the S. pyogenes sgRNA (black arrows) renders the sgRNA self-targeting, as evidenced by cleavage of PCR amplicons into two fragments (380 bp and 150 bp) in mod2 sgRNA variant (stgRNA). HEK 293T cell lines expressing each of the variant sgRNAs were transfected with plasmids expressing Cas9 or mYFP. Cells were harvested 96 hours post transfection, and the genomic DNA was PCR amplified and subjected to T7 E1 assays. The gel picture is presented here. FIG. 17C shows further analysis via next-generation-sequencing confirming that the stgRNA can effectively generate mutations at its own DNA locus. HEK293T cells constitutively expressing the stgRNA were transfected with plasmids expressing Cas9 or mYFP. PCR amplified genomic DNA was sequenced via illumina MiSeq and percentage of mutated sequences is presented. Only the Cas9 transfected cells acquired specific mutations at the stgRNA locus whereas the mYFP transfected cells showed a basal level (˜1%) mutation rate corresponding to the next generation sequencing error rate. The error bars represent the s.e.m. of biological duplicates of the experiment. FIG. 17D shows that among mutated sequences, the percentage of specific mutation types (deletion or insertion) occurring at individual base pair position is presented. FIG. 17E shows that computationally designed stgRNAs with longer SDS regions (30nt-1, 40nt-1 and 70nt-1) demonstrate self-targeting activity. HEK293T cells expressing the 30nt-1, 40nt-1 and 70nt-1 were transfected with plasmids expressing Cas9 or mYFP. T7 Endonuclease I assays were performed on the PCR amplified genomic DNA and the gel picture presented. Also see FIG. 21, constructs 1 through 11 in Table 2.

FIGS. 18A-18E depict the tracking of repetitive and continuous self-targeting activity at the stgRNA locus. FIG. 18A is a schematic of the Mutation-Based Toggling Reporter system (MBTR system) with either a stgRNA in the Mutation Detection Region (MDR) or a regular sgRNA target sequence embedded in the MDR region. A table listing the potential read-out of the MBTR system depending on different indel sizes at the MDR is shown. In the self-targeting scenario, a U6 promoter driven stgRNA with a 27nt SDS is embedded between a constitutive human CMV promoter and modified GFP and RFP reporters. RNAP II mediated transcription starts upstream of the U6 promoter. Correct reading frames of each protein relative to the start codon are indicated in the superscript as F1, F2 and F3. Different sizes of indel formation at the stgRNA locus results in different peptides sequences being translated. Two self-cleaving 2A peptides, P2A and T2A, when translated in-frame, will cause splicing of the peptides and release the functional fluorescent protein from the nonsense peptides, thus result in the appropriate fluorescent output signal. The non-self-targeting construct consists of a U6 promoter driving expression of a regular sgRNA, and the MBTR system contains the target sequence of the regular sgRNA as the MDR. FIG. 18B shows an outline illustrating a double sorting experiment to track repetitive self-cleavage activity using the MBTR system. HEK293T cells stably expressing Cas9 (UBCp-Cas9 cells) were infected with MBTR constructs at low titre to ensure single copy integration. Five days after the initial infection, Gen 1 cells are sorted into GFP or RFP positive populations (Gen1:GFP and Gen1:RFP). The genomic DNA is extracted from a portion of the sorted cells. The rest of the sorted cells are allowed to grow to generate further mutations at the stgRNA loci. The cells initially sorted for GFP or RFP fluorescence, (Gen2R and Gen2G) are sorted again 7 days after the first sort. The genomic DNA of the sorted cells (Gen2R:RFP, Gen2R:GFP, Gen2G:RFP and Gen2G:GFP) is collected and sequenced. FIG. 18C shows the microscopy analysis and FIG. 18D shows flow cytometry data before the 1st and 2nd sort of the self-targeting and non self-targeting constructs. FIG. 18E shows the genomic DNA collected from sorted cells is amplified and cloned into E. coli, and subjected to bacterial colony sanger sequencing. Indels observed via sanger sequencing of the cloned, PCR amplified genomic DNA from sorted cells is presented. SEQ ID NOs: 53-67, 57, 57, and 68 appear in this figure from top to bottom, respectively. Also see FIG. 23.

FIGS. 19A-19F depict the stgRNA sequence evolution analysis. FIG. 19A shows the plasmid map schematizes the DNA construct(s) used in building barcode libraries encoding stgRNA loci. A randomized 16p barcode placed immediately downstream of the stgRNA expression cassette is used to tag unique stgRNA loci when integrated in to the genome of UBCp-Cas9 cells. FIG. 19B shows the time course schematic illustrates the experimental workflow undertaken to perform sequence evolution analysis of stgRNA loci. FIG. 19 C show that by lentivirally infecting UBCp-Cas9 cells at ˜0.3 MOI, a single genomic copy of 16 bp barcode tagged stgRNA locus is introduced per each cell. Multiple such transduced cells constitute parallel but independently evolving stgRNA loci. FIG. 19D shows the number of 16 bp barcodes that are associated with any particular 30nt-1 stgRNA sequence variant is plotted for three different time points (day 2, day 6 and day 14). Each unique, aligned sequence (in the ‘MIXD’ format, methods) is identified by an integer index along the x-axis. The starting sequence is indexed by Index #1. FIG. 19E shows a transition probability matrix for the top 100 most frequent sequence variants of the 30nt-1 stgRNA. The color intensity at each (x, y) position in the matrix indicates the likelihood of an stgRNA sequence variant y transitioning to an stgRNA sequence variant x within a sample collection time point (2 days). Since the non-self targeting sequence variants do not participate in self-targeting action, the y-axis is shown to consist only of self-targeting states. The integer index of an stgRNA sequence variant is provided along with a graphical representation of the stgRNA sequence variant wherein a deletion is illustrated using a blank space, an insertion using a red box and an un mutated base pair using a gray box. Left to right and bottom to top, the stgRNA sequence variants are arranged in order of increasing lengths of deletions away from the PAM. FIG. 19F shows percent mutated stgRNA metric plotted for each of the stgRNAs as a function of time. Also see FIGS. 24-29.

FIGS. 20A-20G depict self-targeting CRISPR-Cas as a memory recording device in vitro and in vivo. FIG. 20A shows a schematic of multiplexed doxycycline and IPTG inducible stgRNA cassettes. By introducing small molecule inducible stgRNA expression constructs into UBCp-Cas9 cells which also express TetR and LacI, the stgRNA expression and its self-targeting activity can be regulated by the respective small molecules. Doxycycline regulated stgRNA and the IPTG regulated stgRNA are placed on the same construct to enable multiplexed recording in single cells. FIG. 20B shows the cleavage fragments observed from T7 endonuclease mutation detection assay under independent regulation of doxycycline and IPTG are presented. Briefly, UBCp-Cas9 cells which also express TetR and Lad were transduced with the inducible stgRNA cassette and the cells were grown either in the presence or absence of 500 ng/mL doxycycline and/or 2 mM IPTG. The cells were harvested 96 hrs post induction and PCR amplified genomic DNA was subject to a T7 E1 assay. FIG. 20C shows plasmid constructs used to build a HEK293T derived clonal NFκBp-Cas9 cell line that expresses Cas9 in response to NFκB activation. The 30nt-1 stgRNA construct is placed on a lentiviral backbone which expresses EBFP2 constitutively. FIG. 20D shows in vitro T7 assay testing for TNF-α inducible stgRNA activity of the NFκBp-Cas9 cells. NFκBp-Cas9 cells containing the 30nt-1 stgRNA were grown either in the presence or absence of 1 ng/mL TNFα for 4 days. The genomic DNA was PCR amplified and assayed for the presence of mutations via the T7 E1 assay. FIG. 20E shows NFκBp-Cas9 cells containing the 30nt-1 stgRNA were grown in media containing different amounts of TNF-α or no TNF-α and cell samples were collected at 36 hr time points for each of the concentrations. Genomic DNA from the samples was PCR amplified, sequenced via next generation sequencing and the percent mutated stgRNA metric was calculated. FIG. 20F shows the experimental outline of the acute inflammation memory recorder in a living animal. Stable NFκBp-Cas9 cells containing the 30nt-1 stgRNA construct were implanted in the flank of three cohorts of four mice each. The three different cohorts of mice were treated either with one or two dosage(s) of LPS on days 7 and 10 or no LPS. After harvesting the samples on day 13 and PCR amplifying the genomic DNA followed by next-generation sequencing analysis, the percent mutated stgRNA metric was calculated. FIG. 20G shows the percent mutated stgRNA metric calculated for the three cohorts of four mice is presented. The height of the dark bar represents the mean while the error bars represent the s.e.m for four mice each. Also see FIGS. 29-33.

FIG. 21 depicts Sanger sequencing of stgRNA locus confirming self-targeted activity. The stgRNA locus was amplified from the genomic DNA extracted via PCR. The purified PCR product was then digested by two restriction enzymes (NheI and KnpI) and cloned in to a bacterial plasmid, which was then transformed into E. coli. Bacterial colonies was picked next day and sequenced. The above indel formations were detected at the stgRNA loci. See also FIGS. 17C, 17D.

FIG. 22 depicts validation of the functionality of MBTR system with different mutation sizes at the MDR. We built constructs with stgRNAs containing indel mutations of sizes (−1 bp and −2 bp). The plasmids were transduced into HEK293T cells that do not express Cas9 and the expected correspondence between indel sizes and fluorescent outputs as shown in the flow cytometry analysis were observed, further confirmed with the fluorescent microscopy imaging. Also see FIG. 18A.

FIGS. 23A-23B depict Sanger Sequencing of stgRNA locus of sorted cells expressing Mutation based toggling reporter system. HEK293T cells stably expressing Cas9 (UBCp-Cas9 cells) were transduced with MBTR construct. After 5 days, cells were sorted into RFP and GFP positive cells (Gen1:RFP and Gen1:GFP). The genomic DNA was extracted from the half of the sorted cells, and the stgRNA locus were amplified and cloned into E. coli. Individual bacterial colonies were then sequenced via Sanger sequencing. (refer to methods). The other half of the sorted cell were allowed to grow and after a week from the initial sort, the cells were sorted again. The stgRNA loci of the harvested cells (Gen2R:RFP, Gen2R:GFP, Gen2G:RFP and Gen2G:GFP) were sequenced accordingly. FIG. 23A shows the sanger sequencing data of each cell population is shown in the figure above. FIG. 23B shows a summary of the percentage match between the observed stgRNA sequence variant and the corresponding fluorescent phenotype.

FIG. 24 depicts workflow illustrating the computational analysis employed in FIG. 19. Illumina NextSeq paired end reads for each of the six stgRNAs (20nt-1, 20nt-2, 30nt-1, 30nt-2, 40nt-1, 40nt-2) was assembled using PEAR (1). For each of the stgRNAs, assembled reads were binned in to different time points after de-multiplexing using 8 bp indexing barcodes. The time point specific reads were then aligned to the reference DNA sequence using the SS2 affine-cost gap algorithm (2) implemented in C++.

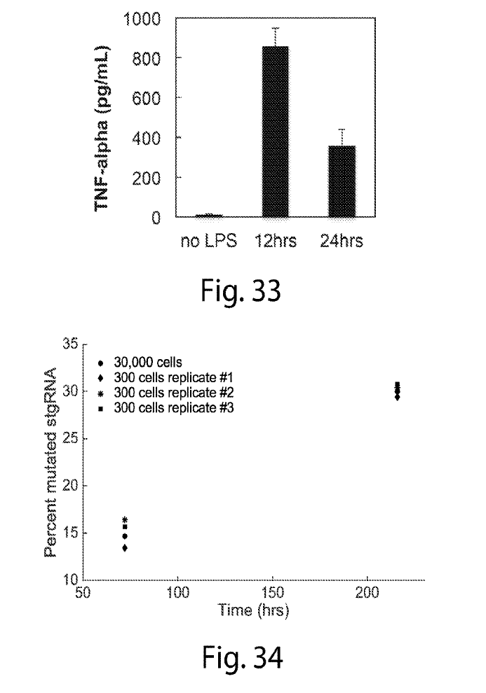

After aligning the sequences with the reference, 16 bp barcodes and the potentially modified upstream stgRNA sequences were extracted. The aligned sequences were represented using words comprised of a four-letter alphabet in the ‘MIXD’ format where ‘M’ represents a match, ‘I’ an insertion, ‘X’ a mismatch and ‘D’ a deletion (FIG. 24). Transition probabilities were computed using sequences belonging to the same barcode but consecutive time points. For each unique sequence variant in a future time point, a unique sequence variant bearing the least hamming distance from the immediate previous time point is assigned a parent. For computing transition probabilities across sequence variants, only the 16 bp barcodes that were represented across all the time points for each of the stgRNAs were considered. A cumulative score of parent-daughter associations is calculated across all barcodes and consecutive time points. Finally, to be a considered a true measure of probability, transition probabilities were normalized to sum to one.

The percent mutated stgRNA metric was computed from the above aligned sequences as the percentage fraction of sequences that contain mutations in the SDS encoding region amongst all the sequences that contain an intact PAM.

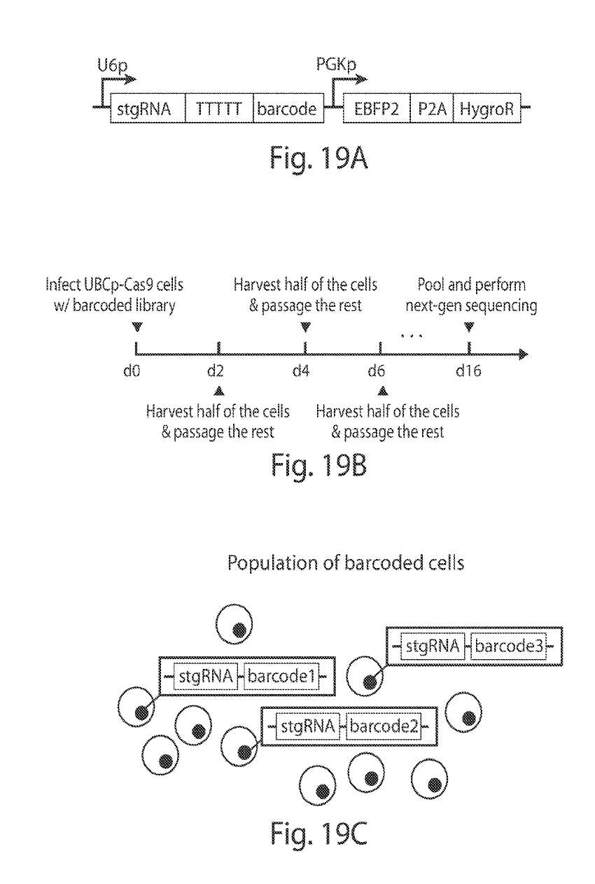

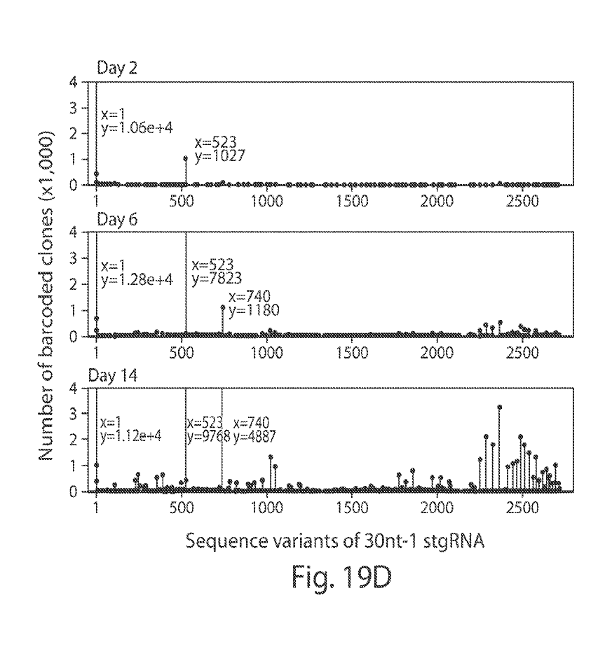

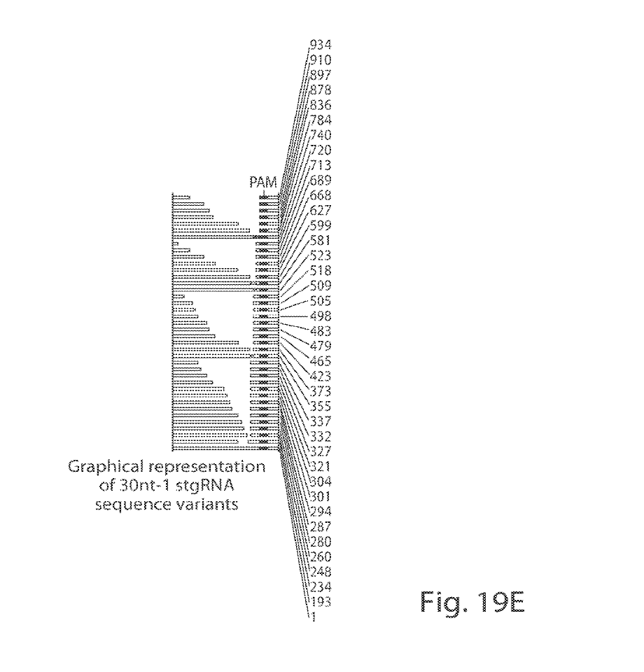

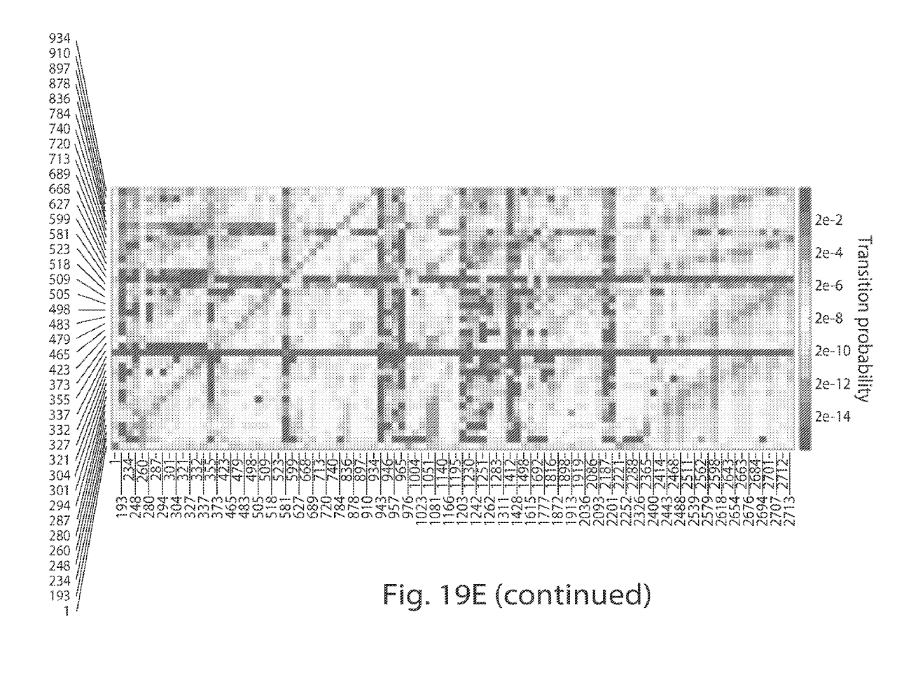

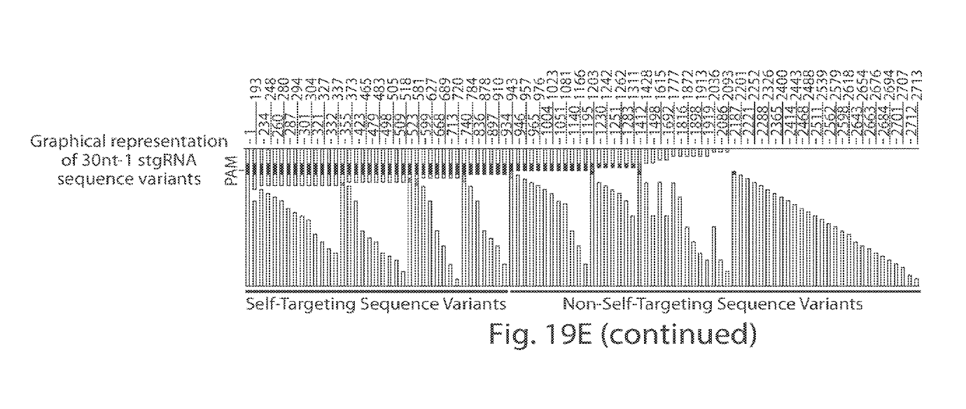

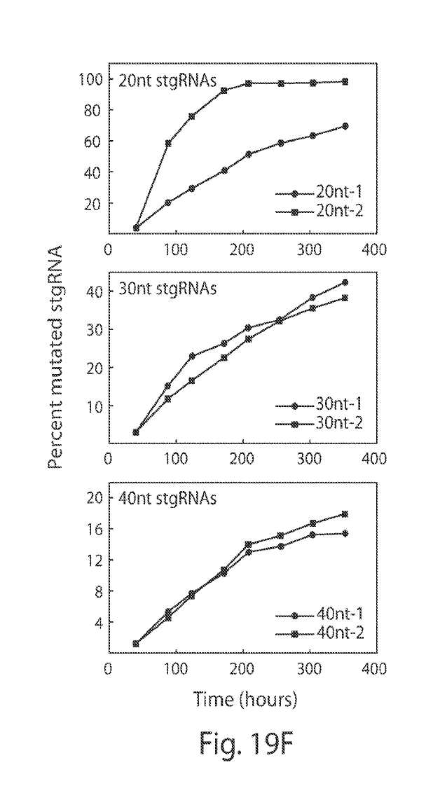

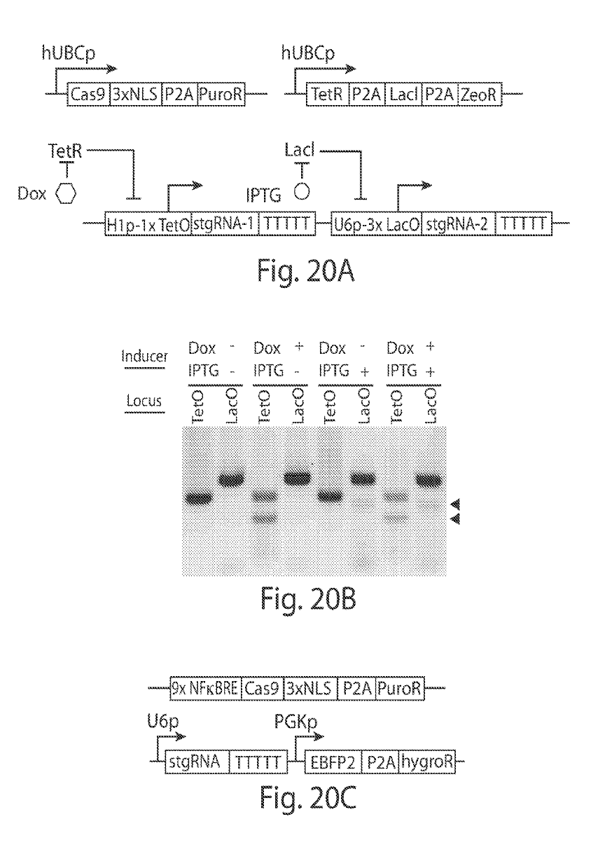

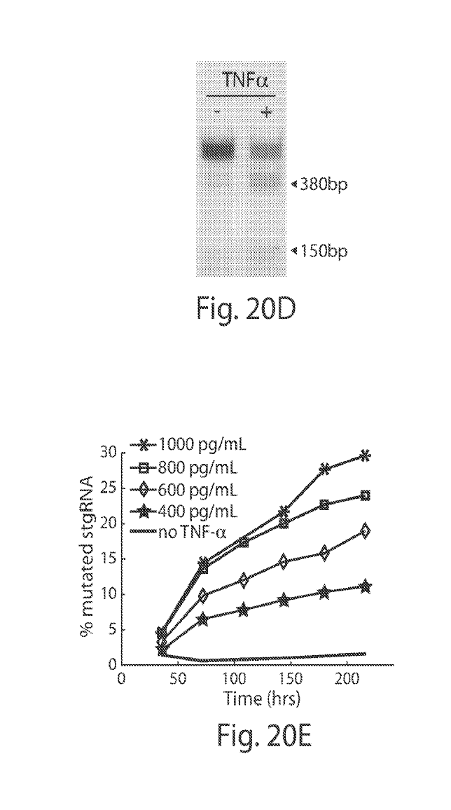

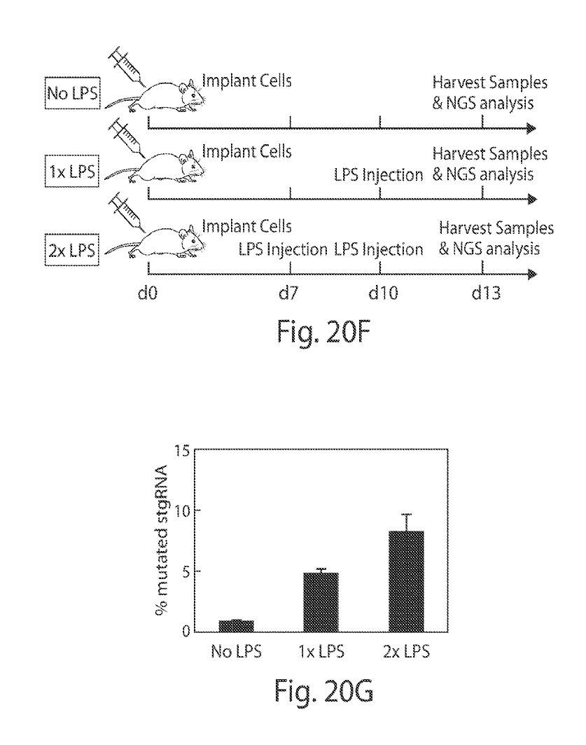

FIG. 25 depicts the top 7 most frequent 30nt-1 stgRNA sequence variants from three different experiments. After aligning the next generation sequencing reads to the reference DNA sequence, sequence variants of the 30nt-1 stgRNA were extracted and represented in the ‘MIXD’ format. A 37 letter word is used to represent the 30nt-1 stgRNA sequence variants where the 37 letters correspond to the first 30 bp of the SDS encoding region, followed by 3 bp of PAM and 4 bp of region encoding the stgRNA handle. The sequence variants presented above are the top 7 most frequently observed sequence variants of 30nt-1 stgRNA for three different experiments performed using two different HEK293T derived cell lines in two different contexts (in vitro or in vivo). A randomly chosen index (from 1 to 2715 in total) is assigned to denote each sequence variant of the 30nt-1 stgRNA. Six sequence variants highlighted above appear with in the list of top 7 sequence variants of the three different experiments. Also see FIGS. 19F, 20E and 20G

FIG. 26 the total number of stgRNA sequence variants in the ‘MIXD’ format observed for 20nt-1, 20nt-2, 30nt-1, 30nt-2, 40nt-1 and 40nt-2 stgRNAs in the barcoded stgRNA evolution experiment. The total number of observed sequence variants in the ‘MIXD’ format composed from all time points and barcodes are presented above for each of the stgRNA loci. The numbers with in the intersecting regions of the Venn diagrams are the number of sequence variants that are observed in common amongst 20nt-1 and 20nt-2 or 30nt-1 and 30nt-2 or 40nt-1 and 40nt-2 stgRNA loci. The numbers in the non-intersecting regions are the sequence variants observed specifically with the respective stgRNA loci. Also see FIG. 19D.

FIG. 27 depicts aligned sequences for two representative barcoded loci for the 30nt-1 stgRNA. For each barcode and each time point, unique sequence variants were identified. The parenthesis at the end of each of the sequence variants indicates the number of reads observed for that variant for the particular time point associated with the specific barcode. Two representative barcodes are presented above.

FIG. 28 depicts transition probability matrix for 30nt-1 stgRNA. In the plot, sequence variants are arranged such that the number of deletions in the sequence variant increases along the x or the y axis. The highlighted features Feature 1 and Feature 2 convey characteristic aspects of 30nt-1 stgRNA sequence evolution. In Feature 1, the transition probability values for transitions along the diagonal are higher than those that are off-diagonal, implying that the 30nt-1 stgRNA variants do not mutagenize much over a 48 hr time point. It was also observed that the transition probability values in the lower triangle (below the diagonal) are higher than the ones in the upper triangle (above the diagonal). This implies that 30nt-1 stgRNA sequence variants have a higher propensity to progressively gain deletions. In Feature 2, transition probability values are higher along the diagonal values. This implies that each of the mutated, self targeting stgRNA variants mutagenize in to non-self targeting variants by mutagenic events resulting in deletions of the downstream PAM sequences while retaining the upstream SDS encoding regions. It was also observed that that sequence variants containing insertions (highlighted by the red arrows) comparatively have a very narrow range of sequence variants they mutate in to.

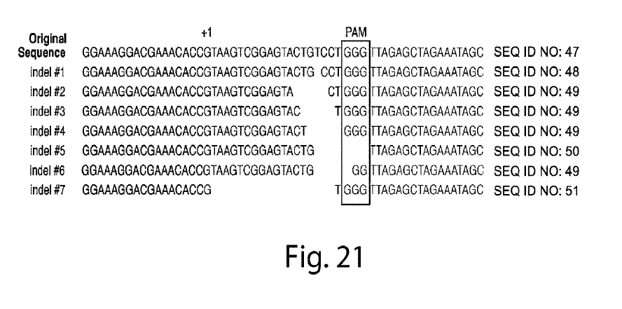

FIGS. 29A-29B depict regular sgRNAs as memory operators. FIG. 21A shows a schematic of the time course experiment in which a regular sgRNA targets a target locus placed downstream. The plasmid map is similar to the one used for building the stgRNA barcode libraries in FIG. 19A. The human U6 promoter drives expression of a regular sgRNA containing either a 20nt-1 or 30nt-2 or 40nt-1 SDS. An sgRNA target locus with its DNA sequence exactly homologous to the SDS and containing a downstream PAM (GGG, the identical PAM used in the sagRNA constructs) is placed 200 bp downstream of the RNAP III terminator ‘TTTTT’. The constructs encoding the 20nt-1, 30nt-2 and 40nt-1 SDSes were cloned in to a lentiviral plasmid backbone harboring a constitutively expressed EBFP2 which is used an infection marker to ensure a target MOI of ˜0.3. For each plasmid construct, ˜200,000 spCas9 cells were infected in separate wells of a 24 well plate on day 0 and cell samples were collected until day 16 at time points roughly spaced 48 hrs apart. At each time point, half of the cell population was harvested and the remaining half was passaged for processing at the next time point. All samples from eight different time points and three different SDSes were pooled together and sequenced in a high throughput fashion via the MiSeq platform. After aligning each of the next generation sequencing reads with the reference DNA sequences, the potentially modified sgRNA target loci were identified and the mutation rate was calculated. FIG. 29B shows the percentage of target sequences mutated is presented as a function of time for 20nt-1, 30nt-2 and 40nt-1 sgRNA target sites.

FIGS. 30A-30B depict small molecule inducible memory operators. By introducing small molecule inducible stgRNA into UBCp-Cas9 cells, the stgRNA expression and its self-targeting activity can be tuned with the respective small molecules. FIG. 29A shows a doxycycline inducible stgRNA construct is built by introducing a Tet operator downstream of a H1 promoter. The doxycycline inducible stgRNA cassette was introduced into UBCp-Cas9 cells also expressing TetR and LacI. The cells were grown in the presence or absence of 500 ng/mL of doxycycline for 5 days and then assayed for self-targeted mutagenesis. The cleavage fragments observed from T7 endonuclease mutation detection assay showed that the stgRNA expression is regulated by doxycycline. Similarly, FIG. 29B shows an IPTG inducible stgRNA construct was built by introducing three copies of Lac operator within the U6 promoter. The IPTG inducible stgRNA cassette was introduced into UBCp-Cas9 cells also expressing TetR and LacI. The cells were grown in the presence or absence of 2 mM IPTG for 5 days and then assayed for self-targeted mutagenesis. In the presence of IPTG, mutations were detected in the stgRNA locus by the T7 E1 assay. Also see FIGS. 20A, 20B and constructs 28-31 Table 2.

FIGS. 31A-31C depict characterization of mKate expression under NF-Kb responsive promoter with and without TNF-alpha stimulation. The mKate expression of HEK293T cell lines stably infected with NF-κB responsive promoter driven mKate construct were quantified. Fluorescence microscopy images of NF-kB responsive stable cell lines with and without TNFα are shown in FIG. 31A. Flow cytometry data show mKate expression histograms for cells under different conditions. FIGS. 31B and 31C show corresponding quantification of the flow cytometry data.

FIGS. 32A-32B depict LPS injection in mice results in elevated mKate expression in cells containing NF-κB responsive mKate reporter. Cells transduced with a NF-kb responsive mKate reporter constructs were implanted in the animal. The construct schematics is shown in FIG. 32A. FIG. 32B shows sample collected 48 hours after the intraperitoneal LPS injection shown significant elevation of mKate expression compare to samples collected from mice did not receive LPS injection.

FIG. 33 depicts tumor Necrosis Factor alpha (TNF-alpha) concentration in serum after LPS injection. After i.p. LPS injection, mice were sacrificed at different points and blood were collected via cardiac puncture. The serum TNF-alpha concentration quantified by mouse TNFα ELISA kit. An elevated TNF-alpha level is observed 12 hours after LPS injection.

FIG. 34 depicts percent mutated stgRNA metric calculated from sequencing genomic DNA corresponding to ˜300 cells, compared with that of 30,000 cells. Genomic DNA was harvested from inflammation recording cells exposed to 1000 pg/mL TNF-α in a 24-well plate. Half of the genomic DNA material (which corresponds to that of 30,000 cells) from the total genomic DNA per well was PCR amplified, sequenced via next generation sequencing and the percent mutated stgRNA metric was calculated and plotted. Three other 1/100 amounts of genomic DNA (corresponding to that of 300 cells) was PCR amplified, sequenced via next generation sequencing and the percent mutated stgRNA metric was also calculated and plotted. Also see FIG. 20E.

### DETAILED DESCRIPTION OF THE INVENTION

Cellular behavior is dynamic, responsive and regulated by the integration of multiple molecular signals. Biological memory devices that can record regulatory events are useful tools for investigating cellular behavior over the course of a biological process and further an understanding of signaling dynamics within cellular niches. Earlier generations of biological memory devices relied on digital switching between two or multiple quasi-stable states based on active transcription and translation of proteins. However, such systems do not maintain their memory after the cells are disruptively harvested. Encoding transient cellular events into genomic DNA memory using DNA recombinases enables the storage of heritable biological information even after gene regulation is disrupted. The capacity and scalability of these memory devices are limited by the number of orthogonal regulatory elements (e.g., transcription factors and recombinases) that can reliably function together. Furthermore, because they are limited to a small number of digital states, they cannot record dynamic (analog) biological information, such as the magnitude or duration of a cellular event. Provided herein, in some embodiments, is an analog memory system that enables the recording of cellular events within human cell populations in the form of DNA mutations by using self-targeting guide RNAs (stgRNAs) to repeatedly mutagenize the DNA that encodes them.

The S. pyogenes Cas9 system from the Clustered Regularly-Interspaced Short Palindromic Repeats-associated (CRISPR-Cas) family is an effective genome engineering enzyme that catalyzes double-stranded breaks and generates mutations at DNA loci targeted by a small guide RNA (sgRNA). The native sgRNA is comprised of a 20 nucleotide (nt) Specificity Determining Sequence (SDS), which specifies the DNA sequence to be targeted, and is immediately followed by a 80 nt scaffold sequence, which associates the sgRNA with Cas9. In addition to sequence homology with the SDS, targeted DNA sequences possess a Protospacer Adjacent Motif (PAM) (5′-NGG-3′) immediately adjacent to their 3′-end in order to be bound by the Cas9-sgRNA complex and cleaved. When a double-stranded break is introduced in the target DNA locus in the genome, the break is repaired by either homologous recombination (when a repair template is provided) or error-prone non-homologous end joining (NHEJ) DNA repair mechanisms, resulting in mutagenesis of targeted locus. Even though the normal DNA locus encoding the sgRNA sequence is perfectly homologous to the sgRNA, it is not targeted by the standard Cas9-sgRNA complex because it does not contain a PAM.

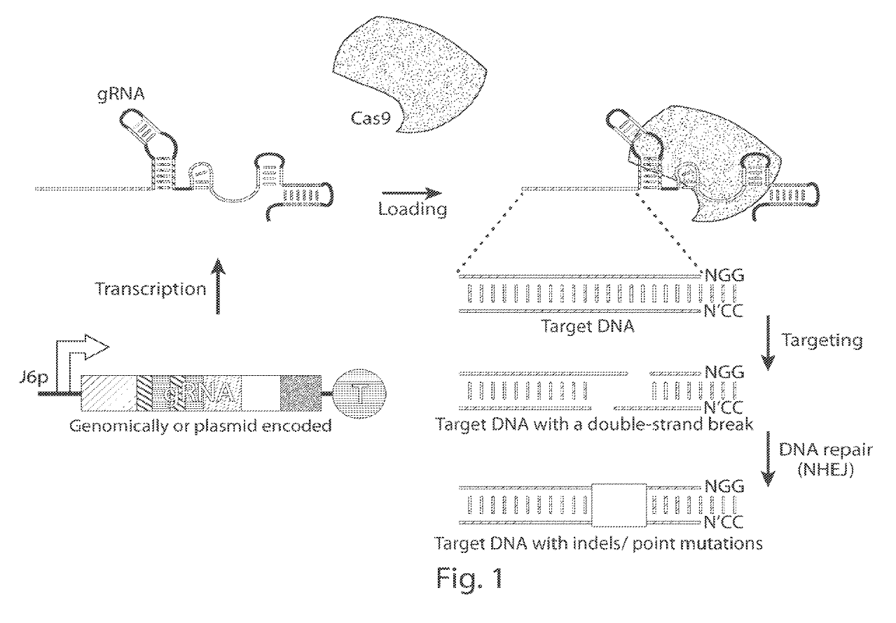

In a wild-type CRISPR/Cas system, guide RNA (gRNA) is encoded genomically or episomally (e.g., on a plasmid) (FIG. 1). Following transcription, the gRNA forms a complex with Cas9 endonuclease. This complex is then “guided” by the specificity determining sequence (SDS) of the gRNA to a DNA target sequence, typically located in the genome of a cell. For Cas9 to successfully bind to the DNA target sequence, a region of the target sequence must be complementary to the SDS of the gRNA sequence and must be immediately followed by the correct protospacer adjacent motif (PAM) sequence (e.g., “NGG”). Thus, in a wild-type CRISPR/Cas9 system, the PAM sequence is present in the DNA target sequence but not in the gRNA sequence (or in the sequence encoding the gRNA).

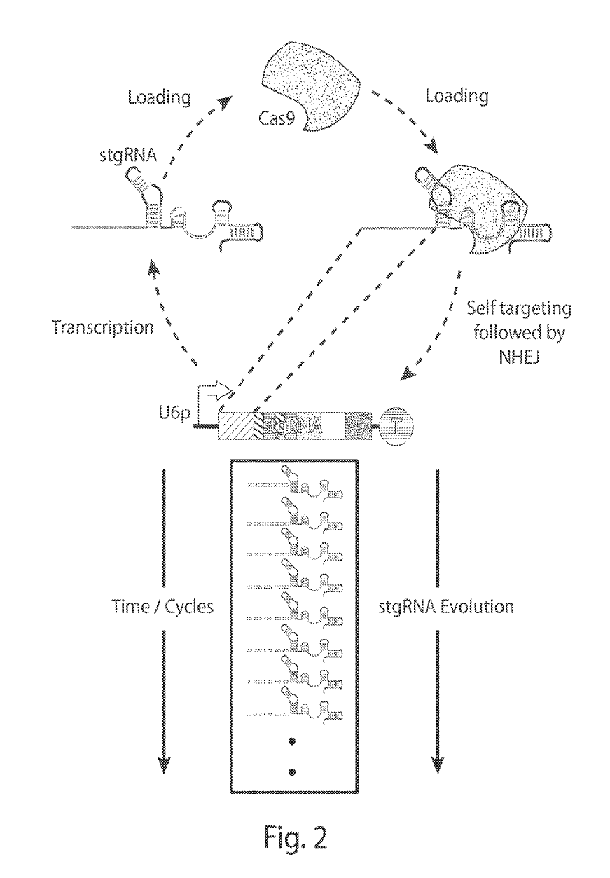

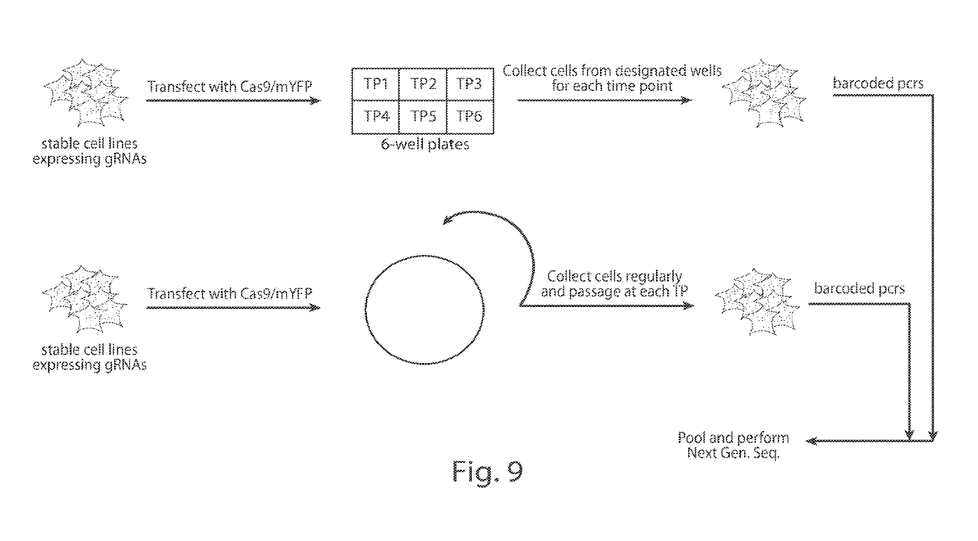

Unlike the wild-type CRISPR/Cas9 system, wherein a gRNA is specific for a single target, the genome editing system of the present disclosure, in some embodiments, provides an iterative self-targeting capability such that a single DNA encoding a gRNA, referred to as “template DNA,” can be used to generate an array of different gRNAs over time (e.g., different from one another). This can be achieved by introducing a PAM sequence into the template DNA, adjacent to an SDS sequence (FIG. 2). As shown in FIG. 9, introduction of a PAM sequence (in this example, “NGG”) into the template DNA resulted in deletions of sequence among different copies of the DNA and, surprisingly, the PAM sequence was preserved in most of copies. This preservation of the PAM sequence permits iterative self-targeting (FIG. 2): the gRNA transcribed from the mutated DNA template containing the PAM sequence and the deleted sequence (referred to herein, in some embodiments, as a self-targeting guide RNA (stgRNA)) complexes with Cas9 and binds to that mutated DNA template from which the stgRNA was transcribed. Cas9 then cleaves the mutated DNA template, creating additional deletions (or insertions). Subsequent transcription of the template produces in a new array of different stgRNAs, each capable of targeting (“self-targeting”) the template DNA from which it was transcribed. This process continues in an iterative manner, allowing for, for example, a form of “continuous evolution.”

In a wild-type CRISPR/Cas system, a gRNA/Cas9 complex does not target the DNA sequences from which the gRNAs are transcribed, the gRNA sequences are not actively modified by CRISPR/Cas, and transcription of the gRNAs within the cell is not required. By contrast, in the self-targeting system of the present disclosure, a gRNA/Cas9 complex targets the DNA sequence from which the gRNAs are transcribed, the gRNA sequences are typically modified by CRISPR/Cas in a targeted fashion, and the gRNAs are transcribed within the cell.

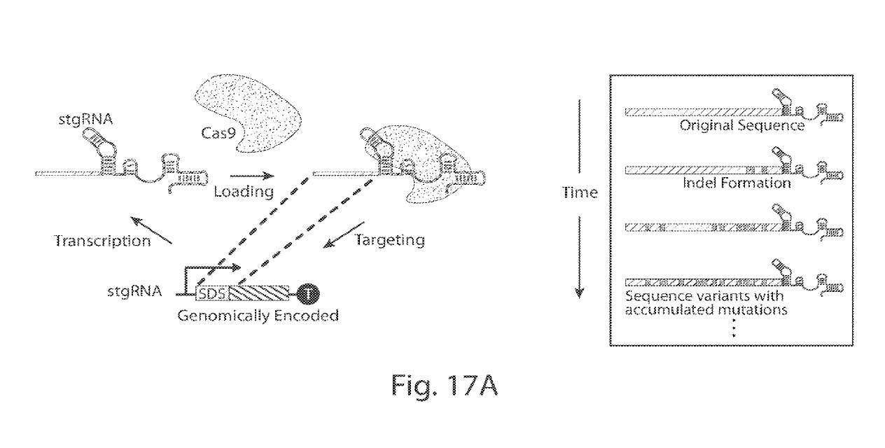

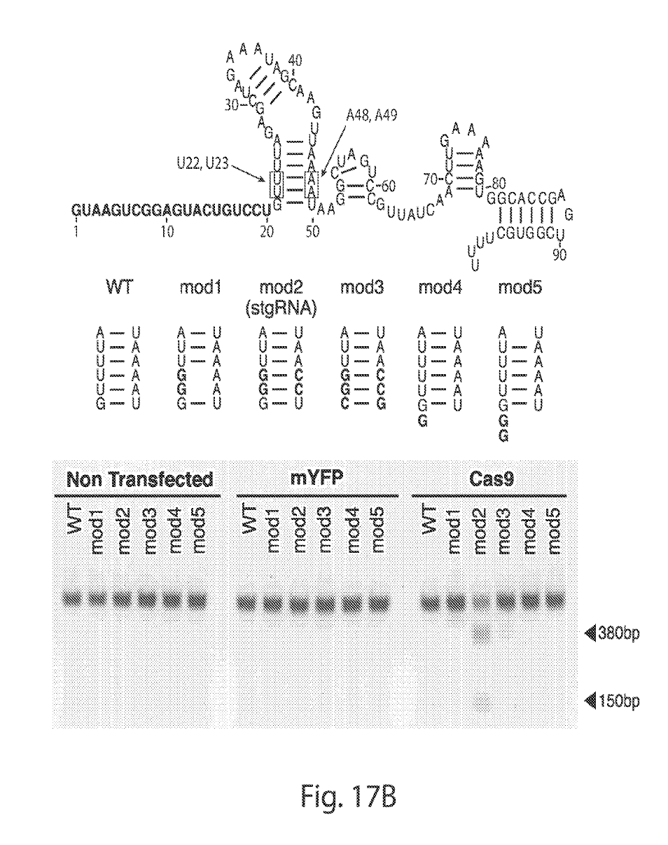

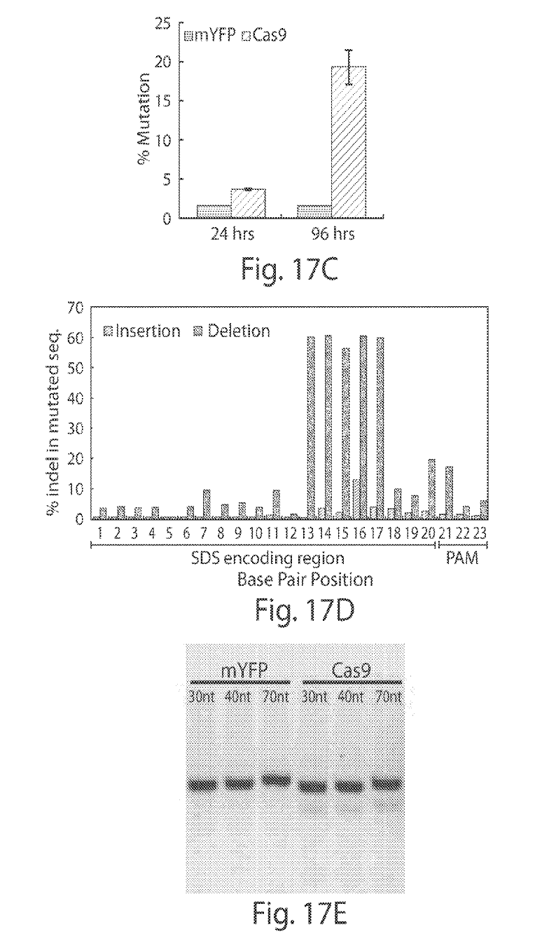

To enable continuous encoding of population-level memory in human cells, modular memory units that can be repeatedly written to generate new sequences and encode additional information over time are provided herein, in some embodiments. With a standard CRISPR-Cas9 system, once a genomic DNA target is repaired, resulting in a novel DNA sequence, it is unlikely to be targeted again by the original sgRNA, because the novel DNA sequence and the sgRNA would lack the necessary sequence homology. By contrast, provided herein is sgRNA architecture engineered so that it acts on the same DNA locus from which the sgRNA is transcribed, rather than a separate sequence elsewhere in the genome, yielding a self-targeting guide RNA (stgRNA) that repeatedly targets and mutagenizes the DNA that encodes it. This was achieved, in some instances, by modifying the DNA sequence from which a sgRNA is transcribed to include a 5′-NGG-3′ PAM immediately downstream of the region encoding the SDS such that the resulting PAM-modified stgRNA would direct Cas9 endonuclease activity towards the stgRNA's own DNA locus. After a double-stranded DNA break is introduced in the SDS and repaired via the NHEJ repair pathway, the resulting de novo mutated stgRNA locus continues to be transcribed as a mutated version of the original stgRNA and participates in another cycle of self-targeting mutagenesis. Multiple cycles of transcription followed by cleavage and error-prone repair occurs, resulting in a self-evolving Cas9-stgRNA system (see, e.g., FIG. 17A). By biologically linking the activity of this system with regulatory events of interest, the DNA locus encoding the stgRNA serves as a memory device that records information in the form of DNA mutations.

Thus, some aspects of the present disclosure are directed to an engineered nucleic acid comprising a promoter operably linked to a nucleotide sequence encoding a guide ribonucleic acid (gRNA) that comprises a specificity determining sequence (SDS) and a protospacer adjacent motif (PAM).

A gRNA is a component of the CRISPR/Cas system. A “gRNA” (guide ribonucleic acid) herein refers to a fusion of a CRISPR-targeting RNA (crRNA) and a trans-activation crRNA (tracrRNA), providing both targeting specificity and scaffolding/binding ability for Cas9 nuclease. A “crRNA” is a bacterial RNA that confers target specificity and requires tracrRNA to bind to Cas9. A “tracrRNA” is a bacterial RNA that links the crRNA to the Cas9 nuclease and typically can bind any crRNA. The sequence specificity of a Cas DNA-binding protein is determined by gRNAs, which have nucleotide base-pairing complementarity to target DNA sequences. Thus, Cas proteins are “guided” by gRNAs to target DNA sequences. The nucleotide base-pairing complementarity of gRNAs enables, in some embodiments, simple and flexible programming of Cas binding. Nucleotide base-pair complementarity refers to distinct interactions between adenine and thymine (DNA) or uracil (RNA), and between guanine and cytosine. In some embodiments, a gRNA is referred to as a stgRNA. A “stgRNA” is a gRNA that complexes with Cas9 and guides the stgRNA/Cas9 complex to the template DNA from which the stgRNA was transcribed.

The length of a gRNA may vary. In some embodiments, a gRNA has a length of 20 nucleotides to 200 nucleotides, or more. For example, a gRNA may have a length of 20 to 175, 20 to 150, 20 to 100, 20 to 95, 20 to 90, 20 to 85, 20 to 80, 20 to 75, 20 to 70, 20 to 65, 20 to 60, 20 to 55, 20 to 50, 20 to 45, 20 to 40, 20 to 35, or 20 to 30 nucleotides.

A “specificity determining sequence,” (SDS) is a nucleotide sequence present in template DNA (e.g., located episomally) or in a target DNA sequence (e.g., located genomically) that is complementary to a region of a gRNA. Typically, a SDS is perfectly (100%) complementary to a region of a gRNA, although, in some embodiments, the SDS may be less than perfectly complementary to a region of a gRNA. For example, the SDS may be 90%, 91%, 92%, 93%, 94%, 95%, 96%, 97%, 98%, or 99% complementary to a region of a gRNA. In some embodiments, the SDS of template DNA or target DNA may differ from a complementary region of a gRNA by 1, 2, 3, 4 or 5 nucleotides.

In some embodiments, an SDS has a length of 15 to 100 nucleotides, or more. For example, an SDS may have a length of 15 to 90, 15 to 85, 15 to 80, 15 to 75, 15 to 70, 15 to 65, 15 to 60, 15 to 55, 15 to 50, 15 to 45, 15 to 40, 15 to 35, 15 to 30, or 15 to 20 nucleotides. In some embodiments, the SDS has a length of 20 nucleotides. In some embodiments, the SDS has a length of 70 nucleotides. In some embodiments, the SDS has a length of 15, 16, 17, 18, 19, 20, 21, 22, 23, 24, or 25 nucleotides. In some embodiments, the SDS has a length of 70 nucleotides. In some embodiments, the SDS has a length of 60, 61, 62, 63, 64, 65, 66, 67, 68, 69, 70, 71, 72, 73, 74 or 75 nucleotides.

A “protospacer adjacent motif” (PAM) is typically a sequence of nucleotides located adjacent to (e.g., within 10, 9, 8, 7, 6, 5, 4, 3, 3, or 1 nucleotide(s) of) an SDS sequence). A PAM sequence is “immediately adjacent to” an SDS sequence if the PAM sequence is contiguous with the SDS sequence (that is, if there are no nucleotides located between the PAM sequence and the SDS sequence). In some embodiments, a PAM sequence is a wild-type PAM sequence. Examples of PAM sequences include, without limitation, NGG, NGR, NNGRR(T/N), NNNNGATT, NNAGAAW, NGGAG, and NAAAAC, AWG, CC. In some embodiments, a PAM sequence is obtained from Streptococcus pyogenes (e.g., NGG or NGR). In some embodiments, a PAM sequence is obtained from Staphylococcus aureus (e.g., NNGRR(T/N)). In some embodiments, a PAM sequence is obtained from Neisseria meningitidis (e.g., NNNNGATT). In some embodiments, a PAM sequence is obtained from Streptococcus thermophilus (e.g., NNAGAAW or NGGAG). In some embodiments, a PAM sequence is obtained from Treponema denticola NGGAG (e.g., NAAAAC). In some embodiments, a PAM sequence is obtained from Escherichia coli (e.g., AWG). In some embodiments, a PAM sequence is obtained from Pseudomonas auruginosa (e.g., CC). Other PAM sequences are contemplated.

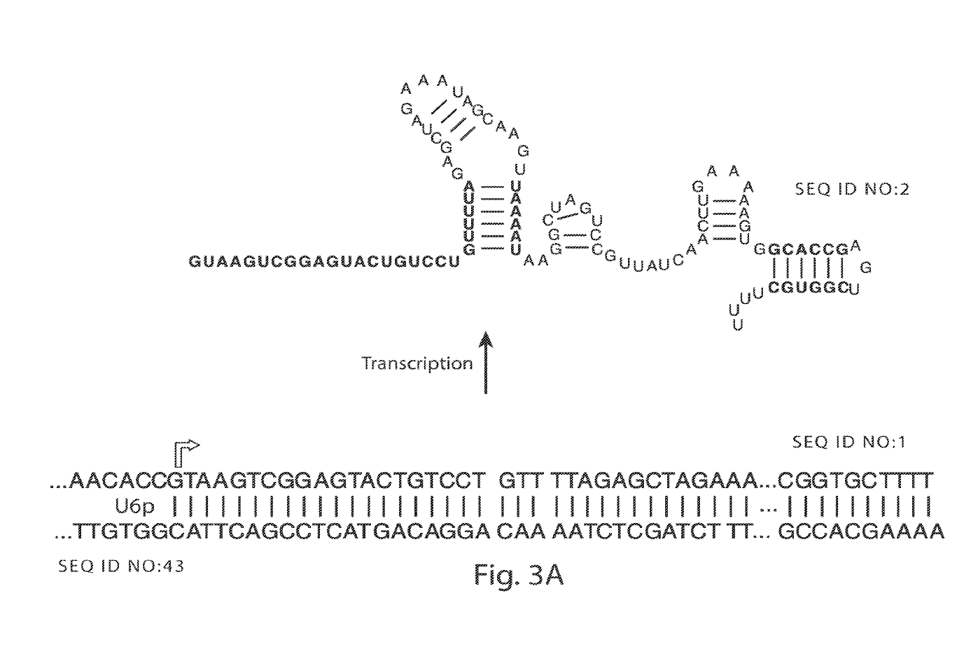

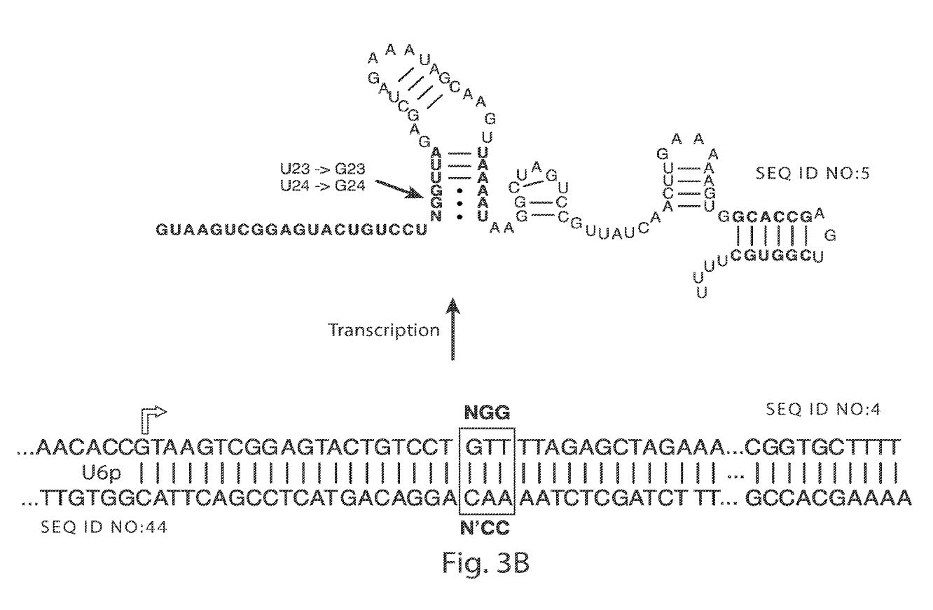

A PAM sequence is typically located downstream (i.e., 3′) from the SDS, although in some embodiments a PAM sequence may be located upstream (i.e., 5′) from the SDS. FIG. 3B shows an example of a PAM sequence (e.g., NGG) located downstream from as SDS (which is located downstream from a U6 promoter sequence, depicted by the arrow).

### Engineered Nucleic Acids

A “nucleic acid” is at least two nucleotides covalently linked together, and in some instances, may contain phosphodiester bonds (e.g., a phosphodiester “backbone”). An “engineered nucleic acid” is a nucleic acid that does not occur in nature. It should be understood, however, that while an engineered nucleic acid as a whole is not naturally-occurring, it may include nucleotide sequences that occur in nature. In some embodiments, an engineered nucleic acid comprises nucleotide sequences from different organisms (e.g., from different species). For example, in some embodiments, an engineered nucleic acid includes a murine nucleotide sequence, a bacterial nucleotide sequence, a human nucleotide sequence, and/or a viral nucleotide sequence. Engineered nucleic acids include recombinant nucleic acids and synthetic nucleic acids. A “recombinant nucleic acid” is a molecule that is constructed by joining nucleic acids (e.g., isolated nucleic acids, synthetic nucleic acids or a combination thereof) and, in some embodiments, can replicate in a living cell. A “synthetic nucleic acid” is a molecule that is amplified or chemically, or by other means, synthesized. A synthetic nucleic acid includes those that are chemically modified, or otherwise modified, but can base pair with naturally-occurring nucleic acid molecules. Recombinant and synthetic nucleic acids also include those molecules that result from the replication of either of the foregoing.

In some embodiments, a nucleic acid of the present disclosure is considered to be a nucleic acid analog, which may contain, at least in part, other backbones comprising, for example, phosphoramide, phosphorothioate, phosphorodithioate, O-methylphophoroamidite linkages and/or peptide nucleic acids. A nucleic acid may be single-stranded (ss) or double-stranded (ds), as specified, or may contain portions of both single-stranded and double-stranded sequence. In some embodiments, a nucleic acid may contain portions of triple-stranded sequence. A nucleic acid may be DNA, both genomic and/or cDNA, RNA or a hybrid, where the nucleic acid contains any combination of deoxyribonucleotides and ribonucleotides (e.g., artificial or natural), and any combination of bases, including uracil, adenine, thymine, cytosine, guanine, inosine, xanthine, hypoxanthine, isocytosine and isoguanine.

Engineered nucleic acids of the present disclosure may include one or more genetic elements. A “genetic element” refers to a particular nucleotide sequence that has a role in nucleic acid expression (e.g., promoter, enhancer, terminator) or encodes a discrete product of an engineered nucleic acid (e.g., a nucleotide sequence encoding a guide RNA, a protein and/or an RNA interference molecule). Examples of genetic elements of the present disclosure include, without limitation, promoters, nucleotide sequences that encode gRNAs and proteins, SDSs, PAMs and terminators.

Engineered nucleic acids of the present disclosure may be produced using standard molecular biology methods (see, e.g., Green and Sambrook, Molecular Cloning, A Laboratory Manual, 2012, Cold Spring Harbor Press).

In some embodiments, engineered nucleic acids are produced using GIBSON ASSEMBLY® Cloning (see, e.g., Gibson, D. G. et al. Nature Methods, 343-345, 2009; and Gibson, D. G. et al. Nature Methods, 901-903, 2010, each of which is incorporated by reference herein). GIBSON ASSEMBLY® typically uses three enzymatic activities in a single-tube reaction: 5′ exonuclease, the 3′ extension activity of a DNA polymerase and DNA ligase activity. The 5′ exonuclease activity chews back the 5′ end sequences and exposes the complementary sequence for annealing. The polymerase activity then fills in the gaps on the annealed regions. A DNA ligase then seals the nick and covalently links the DNA fragments together. The overlapping sequence of adjoining fragments is much longer than those used in Golden Gate Assembly, and therefore results in a higher percentage of correct assemblies.

Also provided herein are vectors comprising engineered nucleic acids. A “vector” is a nucleic acid (e.g., DNA) used as a vehicle to artificially carry genetic material (e.g., an engineered nucleic acid) into another cell where, for example, it can be replicated and/or expressed. In some embodiments, a vector is an episomal vector (see, e.g., Van Craenenbroeck K. et al. Eur. J. Biochem. 267, 5665, 2000, incorporated by reference herein). A non-limiting example of a vector is a plasmid. Plasmids are double-stranded generally circular DNA sequences that are capable of automatically replicating in a host cell. Plasmid vectors typically contain an origin of replication that allows for semi-independent replication of the plasmid in the host and also the transgene insert. Plasmids may have more features, including, for example, a “multiple cloning site,” which includes nucleotide overhangs for insertion of a nucleic acid insert, and multiple restriction enzyme consensus sites to either side of the insert. Another non-limiting example of a vector is a viral vector.

Promoters

Engineered nucleic acids of the present disclosure may comprise promoters operably linked to a nucleotide sequence encoding, for example, a gRNA. A “promoter” refers to a control region of a nucleic acid sequence at which initiation and rate of transcription of the remainder of a nucleic acid sequence are controlled. A promoter may also contain sub-regions at which regulatory proteins and molecules may bind, such as RNA polymerase and other transcription factors. Promoters may be constitutive, inducible, activatable, repressible, tissue-specific or any combination thereof.

A promoter drives expression or drives transcription of the nucleic acid sequence that it regulates. Herein, a promoter is considered to be “operably linked” when it is in a correct functional location and orientation in relation to a nucleic acid sequence it regulates to control (“drive”) transcriptional initiation and/or expression of that sequence.

A promoter may be one naturally associated with a gene or sequence, as may be obtained by isolating the 5′ non-coding sequences located upstream of the coding segment of a given gene or sequence. Such a promoter is referred to as an “endogenous promoter.”

In some embodiments, a coding nucleic acid sequence may be positioned under the control of a recombinant or heterologous promoter, which refers to a promoter that is not normally associated with the encoded sequence in its natural environment. Such promoters may include promoters of other genes; promoters isolated from any other cell; and synthetic promoters or enhancers that are not “naturally occurring” such as, for example, those that contain different elements of different transcriptional regulatory regions and/or mutations that alter expression through methods of genetic engineering that are known in the art. In addition to producing nucleic acid sequences of promoters and enhancers synthetically, sequences may be produced using recombinant cloning and/or nucleic acid amplification technology, including polymerase chain reaction (PCR) (see U.S. Pat. No. 4,683,202 and U.S. Pat. No. 5,928,906).

Contemplated herein, in some embodiments, are RNA pol II and RNA pol III promoters. Promoters that direct accurate initiation of transcription by an RNA polymerase II are referred to as RNA pol II promoters. Examples of RNA pol II promoters for use in accordance with the present disclosure include, without limitation, human cytomegalovirus promoters, human ubiquitin promoters, human histone H2A1 promoters and human inflammatory chemokine CXCL 1 promoters. Other RNA pol II promoters are also contemplated herein. Promoters that direct accurate initiation of transcription by an RNA polymerase III are referred to as RNA pol III promoters. Examples of RNA pol III promoters for use in accordance with the present disclosure include, without limitation, a U6 promoter, a H1 promoter and promoters of transfer RNAs, 5S ribosomal RNA (rRNA), and the signal recognition particle 7SL RNA.

Inducible Promoters

Promoters of an engineered nucleic acids may be “inducible promoters,” which are promoters that are characterized by regulating (e.g., initiating or activating) transcriptional activity when in the presence of, influenced by or contacted by an inducer signal. An inducer signal may be endogenous or a normally exogenous condition (e.g., light), compound (e.g., chemical or non-chemical compound) or protein that contacts an inducible promoter in such a way as to be active in regulating transcriptional activity from the inducible promoter. Thus, a “signal that regulates transcription” of a nucleic acid refers to an inducer signal that acts on an inducible promoter. A signal that regulates transcription may activate or inactivate transcription, depending on the regulatory system used. Activation of transcription may involve directly acting on a promoter to drive transcription or indirectly acting on a promoter by inactivation a repressor that is preventing the promoter from driving transcription. Conversely, deactivation of transcription may involve directly acting on a promoter to prevent transcription or indirectly acting on a promoter by activating a repressor that then acts on the promoter.

The administration or removal of an inducer signal results in a switch between activation and inactivation of the transcription of the operably linked nucleic acid sequence. Thus, the active state of a promoter operably linked to a nucleic acid sequence refers to the state when the promoter is actively regulating transcription of the nucleic acid sequence (i.e., the linked nucleic acid sequence is expressed). Conversely, the inactive state of a promoter operably linked to a nucleic acid sequence refers to the state when the promoter is not actively regulating transcription of the nucleic acid sequence (i.e., the linked nucleic acid sequence is not expressed).

An inducible promoter of the present disclosure may be induced by (or repressed by) one or more physiological condition(s), such as changes in light, pH, temperature, radiation, osmotic pressure, saline gradients, cell surface binding, and the concentration of one or more extrinsic or intrinsic inducing agent(s). An extrinsic inducer signal or inducing agent may comprise, without limitation, amino acids and amino acid analogs, saccharides and polysaccharides, nucleic acids, protein transcriptional activators and repressors, cytokines, toxins, petroleum-based compounds, metal containing compounds, salts, ions, enzyme substrate analogs, hormones or combinations thereof.

Examples of cytokines include, but are not limited to, eotaxin-2, MPIF-2, eotaxin-3, MIP-4-alpha, Fas Fas/TNFRSF6/Apo-1/CD95, FGF-4, FGF-6, FGF-7, FGF-9, Flt-3 Ligand fms-like tyrosine kinase-3, FKN or FK, GCP-2, GCSF, GDNF Glial, GITR, GITR, GM-CSF, GRO, GRO-α, HCC-4, hematopoietic growth factor, hepatocyte growth factor, 1-309, ICAM-1, ICAM-3, IFN-γ, IGFBP-1, IGFBP-2, IGFBP-3, IGFBP-4, IGFBP-6, IGF-I, IGF-I SR, IL-1α, IL-1β, IL-1, IL-1 R4, ST2, IL-3, IL-4, IL-5, IL-6, IL-8, IL-10, IL-11, IL-12 p40, IL-12p70, IL-13, IL-16, IL-17, I-TAC, alpha chemoattractant, lymphotactin, MCP-1, MCP-2, MCP-3, MCP-4, M-CSF, MDC, MIF, MIG, MIP-1α, MIP-1β, MIP-1δ, MIP-3α, MIP-3β, MSP-a, NAP-2, NT-3, NT-4, osteoprotegerin, oncostatin M, PARC, PDGF, P1GF, RANTES, SCF, SDF-1, soluble glycoprotein 130, soluble TNF receptor I, soluble TNF receptor II, TARC, TECK, TGF-beta 1, TGF-beta 3, TIMP-1, TIMP-2, TNF-α, TNF-β, thrombopoietin, TRAIL R3, TRAIL R4, uPAR, VEGF and VEGF-D.

Inducible promoters of the present disclosure include any inducible promoter described herein or known to one of ordinary skill in the art. Examples of inducible promoters include, without limitation, chemically/biochemically-regulated and physically-regulated promoters such as alcohol-regulated promoters, tetracycline-regulated promoters (e.g., anhydrotetracycline (aTc)-responsive promoters and other tetracycline-responsive promoter systems, which include a tetracycline repressor protein (tetR), a tetracycline operator sequence (tetO) and a tetracycline transactivator fusion protein (tTA)), steroid-regulated promoters (e.g., promoters based on the rat glucocorticoid receptor, human estrogen receptor, moth ecdysone receptors, and promoters from the steroid/retinoid/thyroid receptor superfamily), metal-regulated promoters (e.g., promoters derived from metallothionein (proteins that bind and sequester metal ions) genes from yeast, mouse and human), pathogenesis-regulated promoters (e.g., induced by salicylic acid, ethylene or benzothiadiazole (BTH)), temperature/heat-inducible promoters (e.g., heat shock promoters), and light-regulated promoters (e.g., light responsive promoters from plant cells).

Other inducible promoter systems are known in the art and may be used in accordance with the present disclosure.

In some embodiments, inducible promoters of the present disclosure function in prokaryotic cells (e.g., bacterial cells). Examples of inducible promoters for use prokaryotic cells include, without limitation, bacteriophage promoters (e.g. Pls1con, T3, T7, SP6, PL) and bacterial promoters (e.g., Pbad, PmgrB, Ptrc2, Plac/ara, Ptac, Pm), or hybrids thereof (e.g. PLlacO, PLtetO). Examples of bacterial promoters for use in accordance with the present disclosure include, without limitation, positively regulated E. coli promoters such as positively regulated σ70 promoters (e.g., inducible pBad/araC promoter, Lux cassette right promoter, modified lamdba Prm promote, plac Or2-62 (positive), pBad/AraC with extra REN sites, pBad, P(Las) TetO, P(Las) CIO, P(Rhl), Pu, FecA, pRE, cadC, hns, pLas, pLux), σS promoters (e.g., Pdps), σ32 promoters (e.g., heat shock) and σ54 promoters (e.g., glnAp2); negatively regulated E. coli promoters such as negatively regulated σ70 promoters (e.g., Promoter (PRM+), modified lamdba Prm promoter, TetR-TetR-4C P(Las) TetO, P(Las) CIO, P(Lac) IQ, RecA_DlexO_DLacO1, dapAp, FecA, Pspac-hy, pcI, plux-cI, plux-lac, CinR, CinL, glucose controlled, modified Pr, modified Prm+, FecA, Pcya, rec A (SOS), Rec A (SOS), EmrR_regulated, BetI_regulated, pLac_lux, pTet_Lac, pLac/Mnt, pTet/Mnt, LsrA/cI, pLux/cI, LacI, LacIQ, pLacIQ1, pLas/cI, pLas/Lux, pLux/Las, pRecA with LexA binding site, reverse BBa_R0011, pLacI/ara-1, pLacIq, rrnB P1, cadC, hns, PfhuA, pBad/araC, nhaA, OmpF, RcnR), σS promoters (e.g., Lutz-Bujard LacO with alternative sigma factor σ38), σ32 promoters (e.g., Lutz-Bujard LacO with alternative sigma factor σ32), and σ54 promoters (e.g., glnAp2); negatively regulated B. subtilis promoters such as repressible B. subtilis σA promoters (e.g., Gram-positive IPTG-inducible, Xyl, hyper-spank) and σB promoters. Other inducible microbial promoters may be used in accordance with the present disclosure.

In some embodiments, inducible promoters of the present disclosure function in eukaryotic cells (e.g., mammalian cells). Examples of inducible promoters for use eukaryotic cells include, without limitation, chemically-regulated promoters (e.g., alcohol-regulated promoters, tetracycline-regulated promoters, steroid-regulated promoters, metal-regulated promoters, and pathogenesis-related (PR) promoters) and physically-regulated promoters (e.g., temperature-regulated promoters and light-regulated promoters).

### Cells and Cell Expression

Engineered nucleic acids of the present disclosure may be expressed in a broad range of host cell types. In some embodiments, engineered nucleic acids are expressed in bacterial cells, yeast cells, insect cells, mammalian cells or other types of cells.

Bacterial cells of the present disclosure include bacterial subdivisions of Eubacteria and Archaebacteria. Eubacteria can be further subdivided into gram-positive and gram-negative Eubacteria, which depend upon a difference in cell wall structure. Also included herein are those classified based on gross morphology alone (e.g., cocci, bacilli). In some embodiments, the bacterial cells are Gram-negative cells, and in some embodiments, the bacterial cells are Gram-positive cells. Examples of bacterial cells of the present disclosure include, without limitation, cells from Yersinia spp., Escherichia spp., Klebsiella spp., Acinetobacter spp., Bordetella spp., Neisseria spp., Aeromonas spp., Franciesella spp., Corynebacterium spp., Citrobacter spp., Chlamydia spp., Hemophilus spp., Brucella spp., Mycobacterium spp., Legionella spp., Rhodococcus spp., Pseudomonas spp., Helicobacter spp., Salmonella spp., Vibrio spp., Bacillus spp., Erysipelothrix spp., Salmonella spp., Streptomyces spp., Bacteroides spp., Prevotella spp., Clostridium spp., Bifidobacterium spp., or Lactobacillus spp. In some embodiments, the bacterial cells are from Bacteroides thetaiotaomicron, Bacteroides fragilis, Bacteroides distasonis, Bacteroides vulgatus, Clostridium leptum, Clostridium coccoides, Staphylococcus aureus, Bacillus subtilis, Clostridium butyricum, Brevibacterium lactofermentum, Streptococcus agalactiae, Lactococcus lactis, Leuconostoc lactis, Actinobacillus actinobycetemcomitans, cyanobacteria, Escherichia coli, Helicobacter pylori, Selnomonas ruminatium, Shigella sonnei, Zymomonas mobilis, Mycoplasma mycoides, Treponema denticola, Bacillus thuringiensis, Staphylococcus lugdunensis, Leuconostoc oenos, Corynebacterium xerosis, Lactobacillus plantarum, Lactobacillus rhamnosus, Lactobacillus casei, Lactobacillus acidophilus, Streptococcus spp., Enterococcus faecalis, Bacillus coagulans, Bacillus ceretus, Bacillus popillae, Synechocystis strain PCC6803, Bacillus liquefaciens, Pyrococcus abyssi, Selenomonas nominantium, Lactobacillus hilgardii, Streptococcus ferus, Lactobacillus pentosus, Bacteroides fragilis, Staphylococcus epidermidis, Zymomonas mobilis, Streptomyces phaechromogenes, or Streptomyces ghanaenis. “Endogenous” bacterial cells refer to non-pathogenic bacteria that are part of a normal internal ecosystem such as bacterial flora.

In some embodiments, bacterial cells of the invention are anaerobic bacterial cells (e.g., cells that do not require oxygen for growth). Anaerobic bacterial cells include facultative anaerobic cells such as, for example, Escherichia coli, Shewanella oneidensis and Listeria monocytogenes. Anaerobic bacterial cells also include obligate anaerobic cells such as, for example, Bacteroides and Clostridium species. In humans, for example, anaerobic bacterial cells are most commonly found in the gastrointestinal tract.

In some embodiments, engineered nucleic acid constructs are expressed in mammalian cells. For example, in some embodiments, engineered nucleic acid constructs are expressed in human cells, primate cells (e.g., vero cells), rat cells (e.g., GH3 cells, OC23 cells) or mouse cells (e.g., MC3T3 cells). There are a variety of human cell lines, including, without limitation, human embryonic kidney (HEK) cells, HeLa cells, cancer cells from the National Cancer Institute's 60 cancer cell lines (NCI60), DU145 (prostate cancer) cells, Lncap (prostate cancer) cells, MCF-7 (breast cancer) cells, MDA-MB-438 (breast cancer) cells, PC3 (prostate cancer) cells, T47D (breast cancer) cells, THP-1 (acute myeloid leukemia) cells, U87 (glioblastoma) cells, SHSY5Y human neuroblastoma cells (cloned from a myeloma) and Saos-2 (bone cancer) cells. In some embodiments, engineered constructs are expressed in human embryonic kidney (HEK) cells (e.g., HEK 293 or HEK 293T cells). In some embodiments, engineered constructs are expressed in stem cells (e.g., human stem cells) such as, for example, pluripotent stem cells (e.g., human pluripotent stem cells including human induced pluripotent stem cells (hiPSCs)). A “stem cell” refers to a cell with the ability to divide for indefinite periods in culture and to give rise to specialized cells. A “pluripotent stem cell” refers to a type of stem cell that is capable of differentiating into all tissues of an organism, but not alone capable of sustaining full organismal development. A “human induced pluripotent stem cell” refers to a somatic (e.g., mature or adult) cell that has been reprogrammed to an embryonic stem cell-like state by being forced to express genes and factors important for maintaining the defining properties of embryonic stem cells (see, e.g., Takahashi and Yamanaka, Cell 126 (4): 663-76, 2006, incorporated by reference herein). Human induced pluripotent stem cell cells express stem cell markers and are capable of generating cells characteristic of all three germ layers (ectoderm, endoderm, mesoderm).

Additional non-limiting examples of cell lines that may be used in accordance with the present disclosure include 293-T, 293-T, 3T3, 4T1, 721, 9L, A-549, A172, A20, A253, A2780, A2780ADR, A2780cis, A431, ALC, B16, B35, BCP-1, BEAS-2B, bEnd.3, BHK-21, BR 293, BxPC3, C2C12, C3H-10T1/2, C6, C6/36, Cal-27, CGR8, CHO, CML T1, CMT, COR-L23, COR-L23/5010, COR-L23/CPR, COR-L23/R23, COS-7, COV-434, CT26, D17, DH82, DU145, DuCaP, E14Tg2a, EL4, EM2, EM3, EMT6/AR1, EMT6/AR10.0, FM3, H1299, H69, HB54, HB55, HCA2, Hepa1c1c7, High Five cells, HL-60, HMEC, HT-29, HUVEC, J558L cells, Jurkat, JY cells, K562 cells, KCL22, KG1, Ku812, KYO1, LNCap, Ma-Mel 1, 2, 3 . . . 48, MC-38, MCF-10A, MCF-7, MDA-MB-231, MDA-MB-435, MDA-MB-468, MDCK II, MG63, MONO-MAC 6, MOR/0.2R, MRCS, MTD-1A, MyEnd, NALM-1, NCI-H69/CPR, NCI-H69/LX10, NCI-H69/LX20, NCI-H69/LX4, NIH-3T3, NW-145, OPCN/OPCT Peer, PNT-1A/PNT 2, PTK2, Raji, RBL cells, RenCa, RIN-5F, RMA/RMAS, S2, Saos-2 cells, Sf21, Sf9, SiHa, SKBR3, SKOV-3, T-47D, T2, T84, THP1, U373, U87, U937, VCaP, WM39, WT-49, X63, YAC-1 and YAR cells.

Cells of the present disclosure, in some embodiments, are modified. A modified cell is a cell that contains an exogenous nucleic acid or a nucleic acid that does not occur in nature (e.g., an engineered nucleic acid encoding a gRNA). In some embodiments, a modified cell contains a mutation in a genomic nucleic acid. In some embodiments, a modified cell contains an exogenous independently replicating nucleic acid (e.g., an engineered nucleic acid present on an episomal vector). In some embodiments, a modified cell is produced by introducing a foreign or exogenous nucleic acid into a cell. A nucleic acid may be introduced into a cell by conventional methods, such as, for example, electroporation (see, e.g., Heiser W. C. Transcription Factor Protocols: Methods in Molecular Biology™ 2000; 130: 117-134), chemical (e.g., calcium phosphate or lipid) transfection (see, e.g., Lewis W. H., et al., Somatic Cell Genet. 1980 May; 6(3): 333-47; Chen C., et al., Mol Cell Biol. 1987 August; 7(8): 2745-2752), fusion with bacterial protoplasts containing recombinant plasmids (see, e.g., Schaffner W. Proc Natl Acad Sci USA. 1980 April; 77(4): 2163-7), transduction, conjugation, or microinjection of purified DNA directly into the nucleus of the cell (see, e.g., Capecchi M. R. Cell. 1980 November; 22(2 Pt 2): 479-88).

In some embodiments, a cell is modified to express a reporter molecule. In some embodiments, a cell is modified to express an inducible promoter operably linked to a reporter molecule (e.g., a fluorescent protein such as green fluorescent protein (GFP) or other reporter molecule).

In some embodiments, a cell is modified to overexpress an endogenous protein of interest (e.g., via introducing or modifying a promoter or other regulatory element near the endogenous gene that encodes the protein of interest to increase its expression level). In some embodiments, a cell is modified by mutagenesis (e.g., gRNA/Cas9-mediated mutagenesis). In some embodiments, a cell is modified by introducing an engineered nucleic acid into the cell in order to produce a genetic change of interest (e.g., via insertion or homologous recombination).

In some embodiments, an engineered nucleic acid construct may be codon-optimized, for example, for expression in mammalian cells (e.g., human cells) or other types of cells. Codon optimization is a technique to maximize the protein expression in living organism by increasing the translational efficiency of gene of interest by transforming a DNA sequence of nucleotides of one species into a DNA sequence of nucleotides of another species. Methods of codon optimization are well-known.

Engineered nucleic acid constructs of the present disclosure may be transiently expressed or stably expressed. “Transient cell expression” refers to expression by a cell of a nucleic acid that is not integrated into the nuclear genome of the cell. By comparison, “stable cell expression” refers to expression by a cell of a nucleic acid that remains in the nuclear genome of the cell and its daughter cells. Typically, to achieve stable cell expression, a cell is co-transfected with a marker gene and an exogenous nucleic acid (e.g., engineered nucleic acid) that is intended for stable expression in the cell. The marker gene gives the cell some selectable advantage (e.g., resistance to a toxin, antibiotic, or other factor). Few transfected cells will, by chance, have integrated the exogenous nucleic acid into their genome. If a toxin, for example, is then added to the cell culture, only those few cells with a toxin-resistant marker gene integrated into their genomes will be able to proliferate, while other cells will die. After applying this selective pressure for a period of time, only the cells with a stable transfection remain and can be cultured further. Examples of marker genes and selection agents for use in accordance with the present disclosure include, without limitation, dihydrofolate reductase with methotrexate, glutamine synthetase with methionine sulphoximine, hygromycin phosphotransferase with hygromycin, puromycin N-acetyltransferase with puromycin, and neomycin phosphotransferase with Geneticin, also known as G418. Other marker genes/selection agents are contemplated herein.

Expression of nucleic acids in transiently-transfected and/or stably-transfected cells may be constitutive or inducible. Inducible promoters for use as provided herein are described above.

Some aspects of the present disclosure provide cells that comprises 1 to 10 engineered nucleic acids (e.g., engineered nucleic acids encoding gRNAs). In some embodiments, a cell comprises 1, 2, 3, 4, 5, 6, 7, 8, 9, 10 or more engineered nucleic acids. It should be understood that a cell that “comprises an engineered nucleic acid” is a cell that comprises copies (more than one) of an engineered nucleic acid. Thus, a cell that “comprises at least two engineered nucleic acids” is a cell that comprises copies of a first engineered nucleic acid and copies of an engineered second nucleic acid, wherein the first engineered nucleic acid is different from the second engineered nucleic acid. Two engineered nucleic acids may differ from each other with respect to, for example, sequence composition (e.g., type, number and arrangement of nucleotides), length, or a combination of sequence composition and length. For example, the SDS sequences of two engineered nucleic acids in the same cells may differ from each other.

Some aspects of the present disclosure provide cells that comprises 1 to 10 episomal vectors, or more, each vector comprising, for example, an engineered nucleic acids (e.g., engineered nucleic acids encoding gRNAs). In some embodiments, a cell comprises 1, 2, 3, 4, 5, 6, 7, 8, 9, 10 or more vectors.

Also provided herein, in some aspects, are methods that comprise introducing into a cell an (e.g., at least one, at least two, at least three, or more) engineered nucleic acid or an episomal vector (e.g., comprising an engineered nucleic acid). As discussed elsewhere herein, an engineered nucleic acid may be introduced into a cell by conventional methods, such as, for example, electroporation, chemical (e.g., calcium phosphate or lipid) transfection, fusion with bacterial protoplasts containing recombinant plasmids, transduction, conjugation, or microinjection of purified DNA directly into the nucleus of the cell.

### Applications

Molecular Recording and Tracking

In some embodiments, a self-targeting genome editing system of the present disclosure can be used as a DNA recorder for biological event monitoring both in vitro and in vivo. For example, an engineered nucleic acid may comprise an inducible promoter operably linked to the nucleic acid encoding a gRNA that comprises an SDS and a PAM sequence.

In some embodiments, a self-targeting genome editing system can enable long-term population-wide and single-cell molecular recording/tracking both in vitro and in vivo.

In some embodiments, a self-targeting genome editing system is regulated by Cas9 and gRNA expression, each of which can be induced by cellular, molecular, chemical, or optical signals (e.g., gene expression reporter/sensor, cell surface receptor binding, small molecules, ultraviolet light, etc.).

In some embodiments, the duration of exposure and/or amplitude of exposure can be recorded on to the genome and encoded in the content of genetic diversity generated at the gRNA locus (or loci).

In some embodiments, a self-targeting genome editing system of the present disclosure can be extended to perform multi-input recording by utilizing multiple inducible gRNAs in single cells. In some embodiments, a self-targeting genome editing system can serve as a building block to build state machines inside cells to record cell states, and can be easily coupled with other synthetic biology tools.

In some embodiments, a self-targeting genome editing system of the present disclosure can be used for cellular barcoding and lineage tracing in vitro and in vivo. For example, by barcoding each cell with a unique genomic barcode, the self-targeting system can reveal cell lineage map by constructing phylogenetic trees based on the mutated gRNA sequences. Starting from progenitor cells, the self-targeting system can enable building a cell-fate map for single cells in a whole organism, which can be deciphered by analyzing the gRNA sequences.

In some embodiments, a self-targeting system can be used to introduce developmentally timed indels at target genes. For example, the self-targeted RNA only begin to target specific loci after certain developmental events.

Programmable Generation of Genomic Diversity

In some embodiments, a self-targeting genome editing system of the present disclosure can be used for protein engineering and directed evolution, as the system can provide a unique and efficient way to generate large genetic diversity continuously at a specific genetic locus (or loci). The system of the present disclosure can be used in the protein engineering context, for example, to generate wide genetic diversity over time to evolve superior proteins/biomolecules using directed evolution platforms.

In some embodiments, a self-targeting genome editing system may serve as a self-evolving molecular system that can be can be used to select/screen for useful molecular phenotypes.

In some embodiments, a deactivated Cas9 (dCas9) is fused to a DNA cleavage domains such as GIY-YIG homing endonucleases or single chain FokI nucleases so that dCas9 can be targeted to specific DNA loci with cleavage occurring away from the dCas9 binding site to reduce mutations in the dCas9 binding site. This way, generating new variants of stgRNAs that might target other sites in the genome can be avoided. Repeated targeting of the DNA locus can occur with mutagenesis happening at locations distal to the dCas9 binding site, hence serving as a continuous memory register.

In some embodiments, epigenetic strategies for memory storage by fusing DNA methyltransferases or demethylases to dCas9 including DNMT3a, DNMT3b or Tet1 respectively may are used. Programmable memory registers would then be comprised of CpG islands that are targeted by dCas9 fusion proteins to write and erase epigenetic memory by adding or removing methyl groups from the memory registers respectively. In some embodiments, methyl CpG binding proteins (MBPs) in which the methylated DNA binding domain is distinct from the transcriptional repression domain such as Kaiso and MBD1 are used to ‘read’ the epigenetic memory without disruptively harvesting the cells. This can be accomplished, for example, by fusing a transcriptional activation domain such as VP16 or p65 to the MBP and activating the expression of fluorescent proteins placed downstream of the epigenetic memory registers.

In some embodiments, using a ‘based-editing’ approach (A. C. Komor, Y. B. Kim, M. S. Packer, J. A. Zuris, D. R. Liu, Programmable editing of a target base in genomic DNA without double-stranded DNA cleavage. Nature. advance on (2016)) helps avoid issues with using mutagenesis via DNA double strand breaks towards memory storage. By fusing the cytidine deaminase APOBEC1 and Uracil DNA glycosylase inhibitor (UGI) to dCas9, one can effect ‘C’ to ‘T’ transitions in DNA loci without introducing a double stranded break. For example, the memory registers may be comprised of arrays of identical dCas9 target sites containing ‘TC’ repeats. The recording capacity of our system can be potentially increased by increasing the array size of identical ‘TC’ repeat containing target sites.

In addition to recording information, the technology disclosed herein, in some embodiments, may be used for lineage tracing in the context of organogenesis. Embryonic stem cells containing stgRNAs may be allowed to develop in to a whole organism and the resulting lineage relationships between multiple cell-types can be delineated via in situ RNA sequencing.

The self-targeting CRISPR-Cas-based memory described herein are applicable to a broad range of biological settings and can provide unique insights into signaling dynamics and regulatory events in cell populations within living animals.

The present invention is further illustrated by the following Examples, which in no way should be construed as further limiting. The entire contents of all of the references (including literature references, issued patents, published patent applications, and co-pending patent applications) cited throughout this application are hereby expressly incorporated by reference, in particular for the teachings that are referenced herein.

### EXAMPLES

The ability to longitudinally track and record molecular events in vivo provides a unique opportunity to monitor signaling dynamics within cellular niches, and to identify critical factors in orchestrating cellular behavior. A self-contained memory device that enables the recording of molecular stimuli in the form of DNA mutations in human cells is described herein. The memory unit includes a self-targeting guide RNA (stgRNA) cassette that repeatedly directs Streptococcus pyogenes Cas9 nuclease activity towards the DNA that encodes the stgRNA, thereby enabling localized, continuous DNA mutagenesis as a function of stgRNA expression. The temporal sequence evolution dynamics of stgRNAs containing 20, 30 and 40 nucleotide SDSes (Specificity Determining Sequences) were analyzed and a population-based recording metric that conveys information about the duration and/or intensity of stgRNA activity was created. By expressing stgRNAs from engineered, inducible RNA polymerase (RNAP) III promoters, programmable and multiplexed memory storage in human cells triggered by doxycycline and isopropyl β-D-1-thiogalactopyranoside (IPTG) was demonstrated. Finally, it was shown that stgRNA memory units encoded in human cells implanted in mice were able to record lipopolysaccharide (LPS) induced acute inflammation over time. The technology of the present disclosure provides a unique tool for investigating, for example, cell biology in vivo and in situ and drives further applications that leverage continuous evolution of targeted DNA sequences in mammalian cells.

### Example 1

Stable cell lines derived from HEK293T cells expressing different stgRNAs were built by infecting HEK293T cells with lentiviral particles containing the cassette expressing stgRNAs (U6p-stgRNA-PGKp-EBFP2-p2a-hgyroR) in their payload. Successfully transduced cells were selected with hygromycin at 300 mg/ml. Stable cell lines expressing stgRNAs were transfected with a plasmid expressing Cas9 (CMVp-Cas9-3xNLS) or with a control plasmid (expressing mYFP). The genomic DNA was harvested 96 hours post transfection and was PCR amplified in the region encoding the stgRNA. Indels and point mutations introduced onto the DNA encoding the stgRNA were detected via a T7 Endonuclease I (T7 E1A) assay. DNA containing indels and point mutations resulted in multiple bands on the gel.

### Example 2

Stable cell lines derived from HEK293T cells expressing different variants of stgRNAs (mod1, mod3, mod4 and mod5) or the wild type gRNA were transfected with a plasmid expressing Cas9 (CMVp-Cas9-3xNLS) or with a control plasmid (expressing mYFP). The genomic DNA was harvested 96 hours post transfection, was PCR amplified in the region encoding the stgRNA, and T7 Endonuclease I (T7 E1A) assays were performed and reported. Incorporation of the 5′-NGG-3′ PAM motif results in the modification of U23, U24, A48 and A49 nucleotides in each of the variant gRNAs.

The mod1 variant demonstrates robust self-targeting activity as evidenced by the lower size band on the gel. The mod3 variant demonstrates self-targeting activity as well, however at lower efficiency.

### Example 3

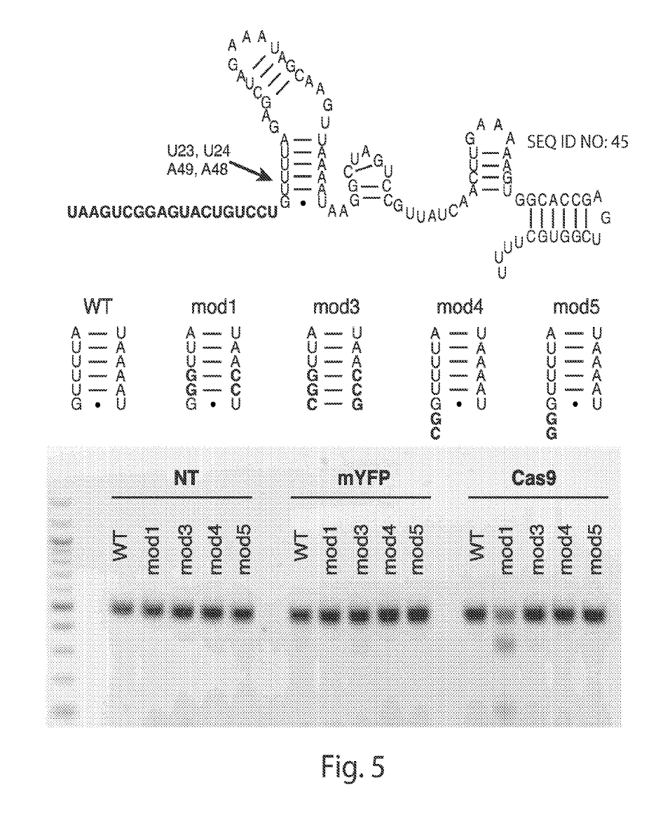

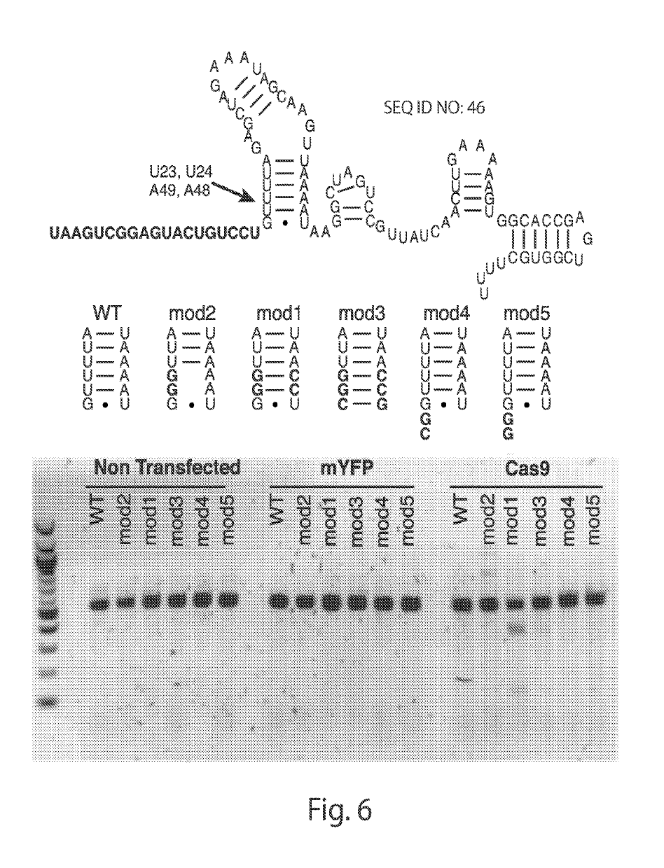

The experimental design is similar to the one in Example 2, FIG. 5. The mod2 variant that contained only the U23→G23 and U24→G24 mutations did not demonstrate self-targeting activity, while the mod1 and mod3 variants that contained additional compensatory A49→C49 and A48→C48 mutations demonstrate self-targeting activity.

### Example 4

Stable cell line expressing stgRNA (mod1 variant) was transfected with plasmids expressing the wild-type Cas9, multiple mis-sense mutant Cas9s, or GFP and was assayed for targeting efficiency via the T7E1 assay 96 hours post transfection. Targeting efficiency calculated from the DNA stain intensity in each gel lane for each of the proteins is also indicated.

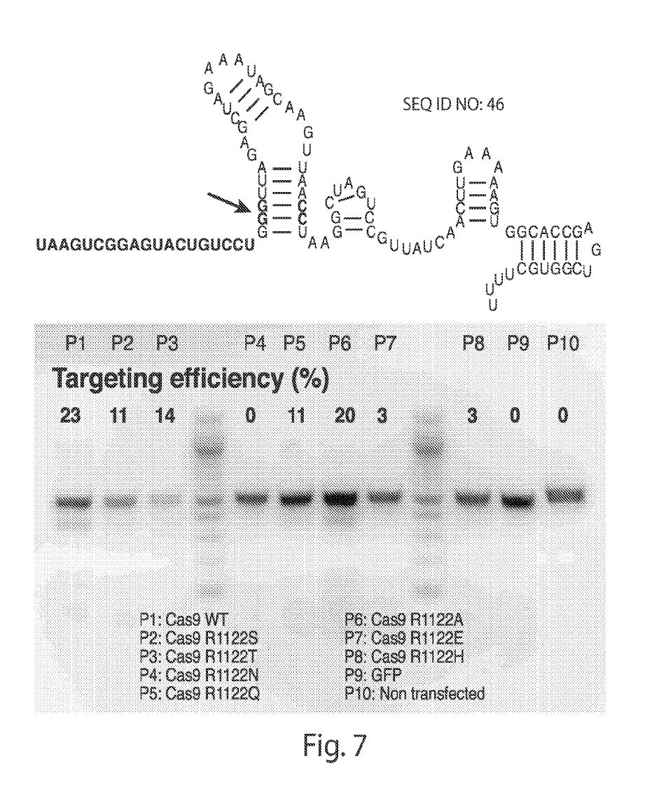

The crystal structure of Cas9 in complex with gRNA and target DNA (Nishimasu H., et. al., Cell 2014) identified that Cas9 amino acid residue Arg1122 stabilizes the lower stem of gRNA by hydrogen bond interactions with U23/A49. FIG. 7 shows results from an assay for the ability of Cas9 containing substitutions of Arg1122 with polar, non-polar and aromatic amino acid residues to enhance self-targeting efficiency missense mutants. The wild-type Cas9 has the highest efficiency of self-targeting activity.

### Example 5

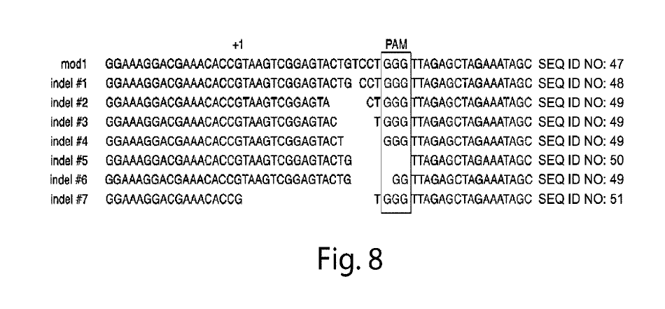

A stable cell line encoding the stgRNA (mod1 variant) was transfected with Cas9. Genomic DNA was harvested 96 hours post transfection, PCR amplified and cloned in to plasmids in E. coli. Individual E. coli colonies were subsequently sanger sequenced, and the modified DNA sequences encoding the stgRNA are shown in FIG. 8. Most of the sequences retain the PAM motif, which enables multiple rounds of self-targeting activity.

### Example 6

Stable cell lines expressing with wild type gRNA or stgRNA were transfected with a plasmid expressing mYFP or Cas9 in two replicates. The experiment was performed in two different configurations—without splitting (FIG. 9A) or with splitting (FIG. 9B).

Without Splitting:

Multiple aliquots of 200,000 cells, each from a larger transfection, were plated in to multiple wells of a six well plate at time 0. The cells were harvested from the corresponding wells for each different time point and barcoded genomic PCRs were performed to extract DNA encoding the stgRNA.

Several different barcoded DNA samples for each time point were pooled along with those from the configuration with splitting and subjected to high throughput sequencing on the MiSeq platform.

With Splitting:

A single aliquot of 200,000 cells was plated at time 0. The cells were harvested at different time points by collecting half of the cell pool and plating the remain half for future time points. Barcoded genomic PCRs were performed to extract DNA encoding the stgRNA, pooled along with the DNA from the configuration without splitting and subjected to high throughput sequencing on MiSeq platform.

### Example 7

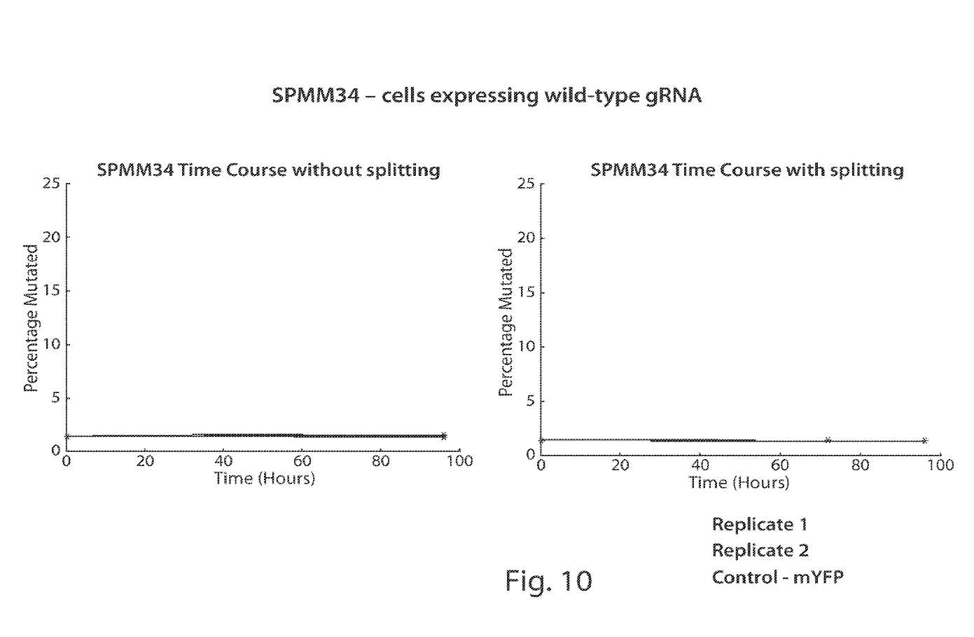

High throughput sequenced data was analyzed for the control cells expressing wild-type gRNA and transfected with a plasmid expressing Cas9 or mYFP. The percentage of gRNA encoding sequences mutated with reference to the unmodified gRNA were plotted as a function of time (FIG. 10). The experiment was performed as described in Example 6, FIG. 10, for two replicates transfected with Cas9 encoding plasmid and one replicate transfected with mYFP expressing plasmid. There were no appreciable mutation of the sequences.

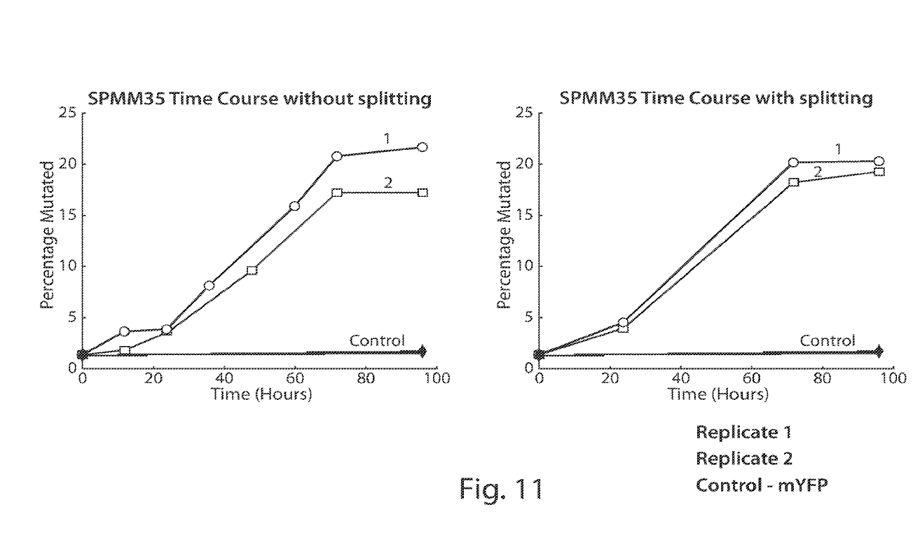

Next, high throughput sequenced data was analyzed for cells expressing stgRNA and transfected with a plasmid expressing Cas9 or mYFP. The percentage of stgRNA encoding sequences mutated with reference to the unmodified gRNA were plotted as a function of time (FIG. 11). The experiment was performed as described above, for two replicates transfected with Cas9 encoding plasmid and one replicate transfected with mYFP expressing plasmid. There was a linear increase in the percentage of mutated sequences as a function of time up until 72 hrs.

### Example 8

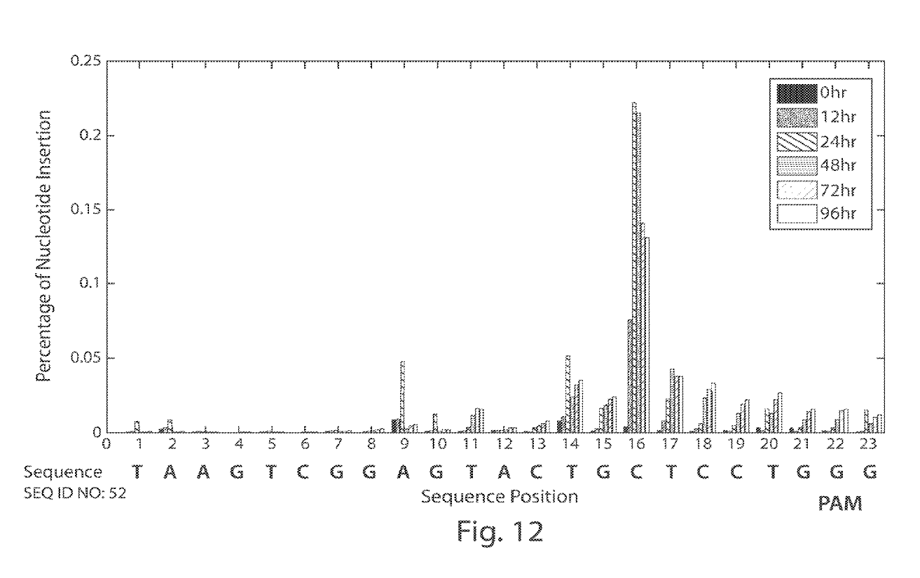

Indel metrics for stgRNA as a function of the base position and time post transfection with Cas9 are plotted in FIG. 12. The 5′-NGG-3′ PAM sequence is located in base positions 21, 22 and 23, while the bases 1 through 20 comprise the 20 bp SDS. The number of insertions observed at each base position normalized to the total number of sequencing reads for each time point is indicated. For each base position, an initial increase in insertion frequency was noticed, reaching a peak at the 24-hour time point, which continued to decrease for further time points. Moreover, there was an increased preference for insertions for bases 14 through 17.

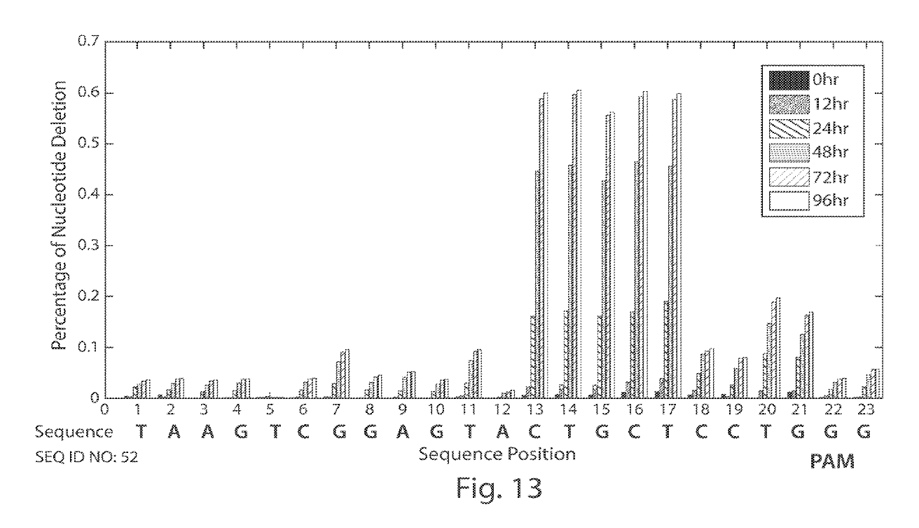

Indel metrics for stgRNA as a function of the base position and time post transfection with Cas9 are plotted in FIG. 13. The 5′-NGG-3′ PAM sequence is located in base positions 21, 22 and 23 while the bases 1 through 20 comprise the 20 base pair (bp) SDS. The number of deletions observed at each base position normalized to the total number of sequencing reads for each time point is indicated. The deletion rate was in general higher than the insertion rate at each base position and continued to increase with time, plateauing at the 72 hour time point. Similar to the bias observed with insertions, there was a marked preference for deletions at bases 13 through 17.

### Example 9

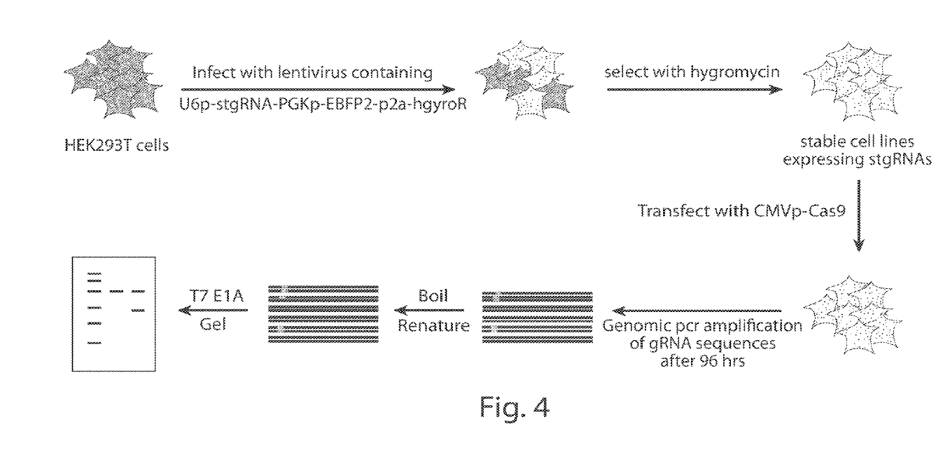

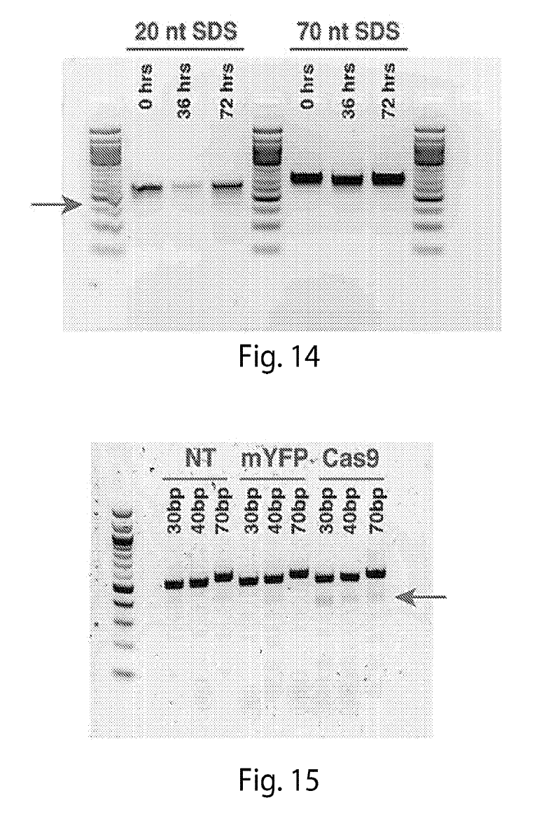

Stable cell lines expressing stgRNAs containing 20 nucleotide (nt) SDS or 70 nt SDS were built similar to the design illustrated in FIG. 4. The 70 nt SDS containing stgRNA was designed by extending the 5′ sequence of the 20 nt SDS containing stgRNA with 50 randomly chosen nucleotides. T7E1 A assays were performed at different time points following transfection with a plasmid expressing Cas9. The arrow indicates the rough estimated size of the product resulting from T7E1 assays of DNA containing indels following self-targeting action (FIG. 14).

There was no (observed) self-targeting activity by the 70 nt SDS containing stgRNA designed by a randomly chosen 50 nt extension of the 20 nt SDS containing stgRNA.

### Example 10

T7E1 assays were conducted using PCR amplified genomic DNA from stable cell lines encoding stgRNAs with computationally designed 30, 40 and 70 nt SDS transfected with plasmids either expressing mYFP or Cas9, 96 hours post transfection. stgRNAs were designed to contain 30, 40 and 70 nt SDS such that they did not fold into any undesired secondary structures while containing the desired nucleotides and secondary structures recognized by Cas9. The Fold software from the ViennaRNA Package was used for this design.

The arrow indicates the estimated size of the product resulting from T7E1 assays of DNA containing indels following self-targeting action (FIG. 15). There was robust self-targeting activity for these computationally designed stgRNAs that contain SDSs of longer lengths.

### Example 11

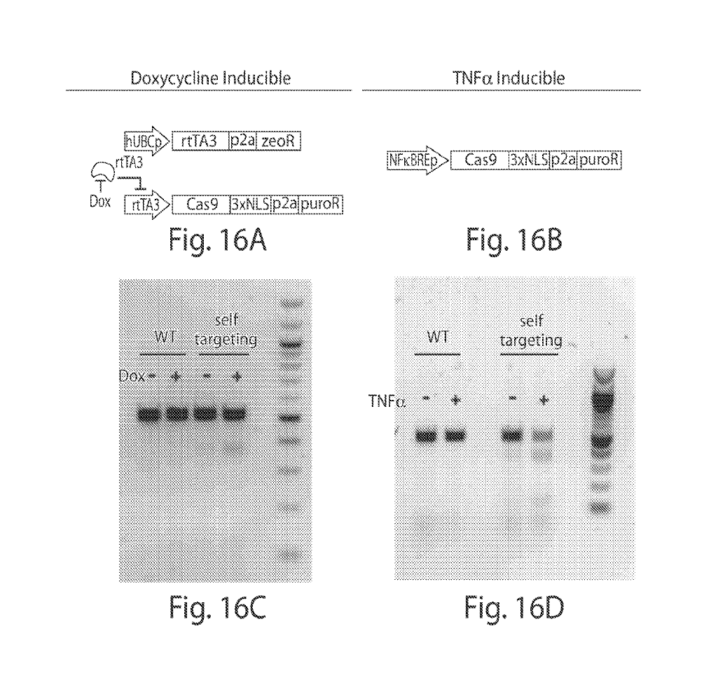

A Dox-inducible Cas9 cell line (FIG. 16A) was transduced with lentiviral vectors (LVs) encoding wild-type gRNA or stgRNA containing 20 nt SDS and induced with or without Dox for 96 hrs. T7E1 assays on PCR-amplified genomic DNA were performed, and gel images are shown in FIG. 16C.

A TNFα inducible Cas9 cell line (FIG. 16B) was transduced with LVs encoding wild-type gRNA or stgRNA containing 20 nt SDS and induced with or without TNFα for 96 hrs. T7E1 assays on PCR-amplified genomic DNA were performed, and gel images are shown in FIG. 16D.

### Example 12

Multiple variants of a S. pyogenes sgRNA-encoding DNA sequence were built with a 5′-GGG-3′ PAM located immediately downstream of the region encoding the 20 nt SDS. The variants were tested for their ability to generate mutations at their own DNA locus. HEK293T-derived stable cell lines were built to express either the wild-type (WT) or each of the variant sgRNAs shown in FIG. 17B (constructs 1-6, SEQ ID NOs: 8-13, Table 2). Plasmids encoding either spCas9 (construct 7, SEQ ID NO: 14, Table 2) or mYFP (negative control) driven by the CMV promoter (CMVp) were transfected into cells stably expressing the depicted sgRNAs, and the sgRNA loci were inspected for mutagenesis using T7 Endonuclease I assays three days after transfection. A straightforward variant sgRNA (mod1) with guanine substitutions at U23 and U24 positions did not exhibit any noticeable self-targeting activity. This was likely due to the presence of bulky guanine and adenine residues facing each other in the stem region, resulting in a de-stabilization of the secondary structure. Thus, compensatory adenosine to cytidine mutations were introduced within the stem region (A48, A49 position) of the mod2 sgRNA variant and robust mutagenesis at the modified sgRNA locus was observed (FIG. 17B). Additional variant sgRNAs (mod3, mod4 and mod5) did not exhibit noticeable self-targeting activity. Thus, the mod2 sgRNA was hereafter used as the stgRNA architecture.

Further, the mutagenesis pattern of the stgRNA was characterized by sequencing the DNA locus encoding it. Cell lines expressing the stgRNA were transfected with a plasmid expressing either Cas9 (construct 7, SEQ ID NO: 14, Table 2) or mYFP driven by the CMV promoter. Genomic DNA was harvested from the cells at either 24 hours or 96 hours post-transfection and subjected to targeted PCR amplification of the region encoding the stgRNAs. The PCR amplicons were either sequenced by MiSeq or cloned into E. coli for clonal Sanger sequencing (FIG. 21). Cells transfected with the Cas9-expressing plasmid exhibited significant mutation frequencies in the stgRNA loci and those frequencies increased over time, compared to cells transfected with the control mYFP expressing plasmid (FIG. 17C). By using high throughput sequencing, the mutated sequences generated by stgRNAs were inspected to determine the probability of insertions or deletions occurring at specific base pair positions (FIG. 17D). Higher rates of deletions were observed compared to insertions at each nucleotide position. Moreover, an elevated percentage of mutated sequences exhibited deletions consecutively spanning nucleotide positions 13-17 for this specific stgRNA (20nt-1). A more thorough analysis was carried out into the sequence evolution patterns of stgRNAs, as described later in FIG. 19.

Given the observation that deletions are preferred over insertions, it was suspected that stgRNAs would be shortened over time with repeated self-targeting, ultimately rendering them ineffective. To enable multiple cycles of self-targeting, stgRNAs that were made up of longer SDSes were designed. A cell line was built initially expressing an stgRNA containing randomly chosen 30 nt SDS (construct 8, SEQ ID NO: 15, Table 2) but no noticeable self-targeting activity was detected when the cell lines were transfected with plasmids expressing Cas9 (data not shown). StgRNAs with longer than 20nt SDSes might contain undesirable secondary structures that result in loss of activity. Therefore, stgRNAs that are predicted to maintain the scaffold fold of regular sgRNAs with out any undesirable secondary structures within the SDS were computationally designed. Stable cell lines encoding stgRNAs containing these computationally designed 30, 40 and 70 nt SDS (constructs 9-11, SEQ ID NOs: 16-18, Table 2) were transfected with a plasmid expressing Cas9 driven by the CMV promoter. T7 Endonuclease I assays of PCR amplified genomic DNA demonstrated robust indel formation in the respective stgRNA loci (FIG. 17E).

### Example 13

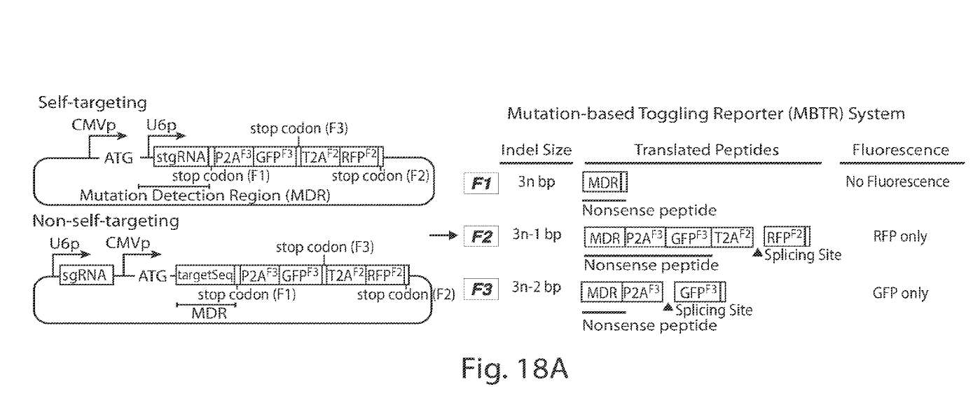

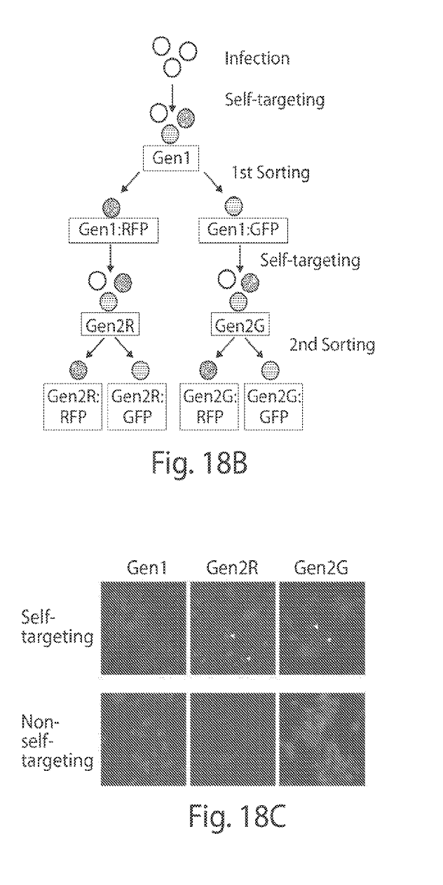

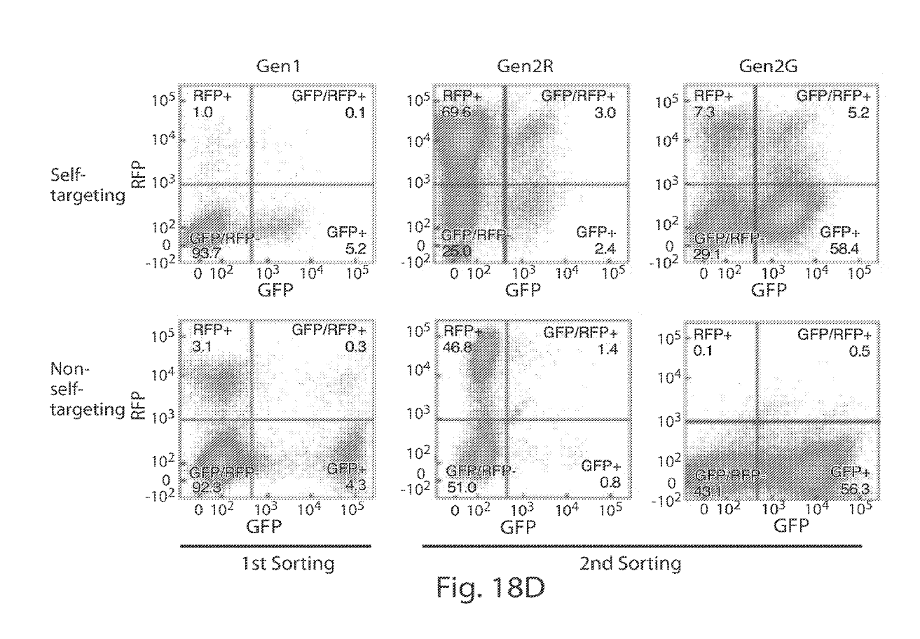

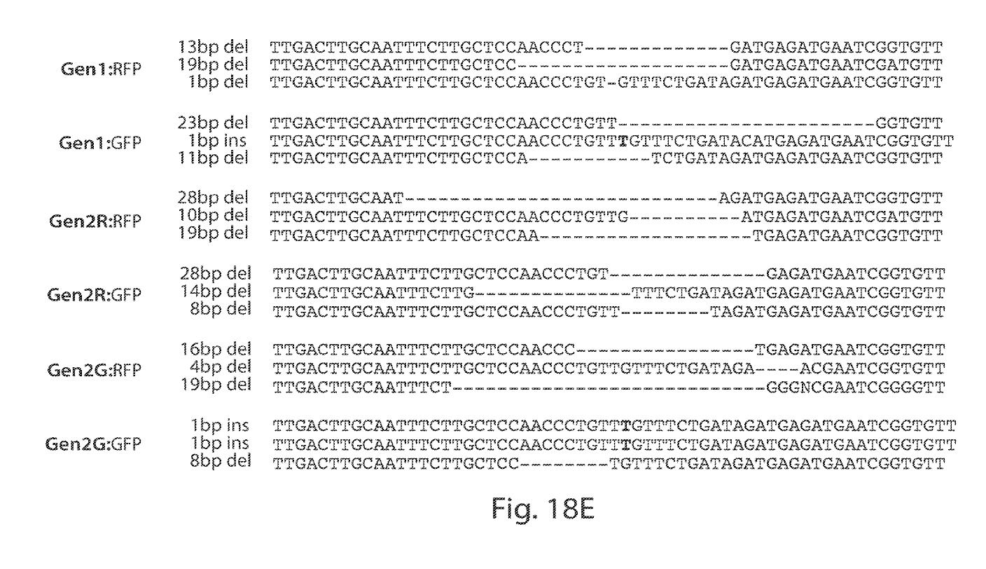

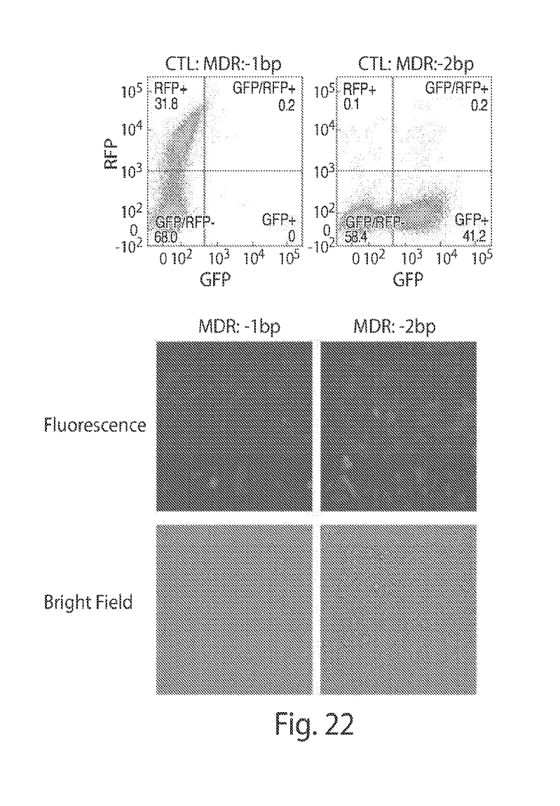

The present disclosure also demonstrates that stgRNA-encoding DNA loci in individual cells undergo multiple rounds of self-targeted mutagenesis. To track genomic mutations in single cells over time, a Mutation-Based Toggling Reporter (MBTR) system that generates distinct fluorescent outputs based on indel sizes at the stgRNA-encoding locus was developed, which was inspired by a design previously described for tracking DNA mutagenesis outcomes. Downstream of a CMV promoter and a canonical ATG start codon, the Mutation Detection Region (MDR) was embedded, which contains a modified U6 promoter followed by a stgRNA. The MDR is immediately followed by out-of-frame green (GFP) and red (RFP) fluorescent proteins, which are separated by ‘2A self-cleaving peptides’ (P2A and T2A) (FIG. 18A, construct 13, SEQ ID NO: 20, Table 2). Different reading frames are expected to be in-frame with the start codon depending on the size of the indels in MDR. In the starting state (reading frame 1, F1), no fluorescence is expected. In reading frame 2 (F2), which corresponds to any −1 bp frameshift mutation, an in-frame RFP is translated along with the T2A self-cleaving peptide, which enables release of the functional RFP from the upstream nonsense peptides. In reading frame 3 (F3) which corresponds to any −2 bp frameshift mutation, GFP is properly expressed downstream of an in-frame P2A and followed with a stop codon. The functionality of this design was confirmed by manually building constructs with stgRNAs containing indel mutations of various sizes (0 bp, −1 bp and −2 bp, constructs 13-15, SEQ ID NOs: 20-22, Table 2), introducing them in to HEK293T cells, and observing the expected correspondence between indel sizes and fluorescent outputs (FIG. 22).

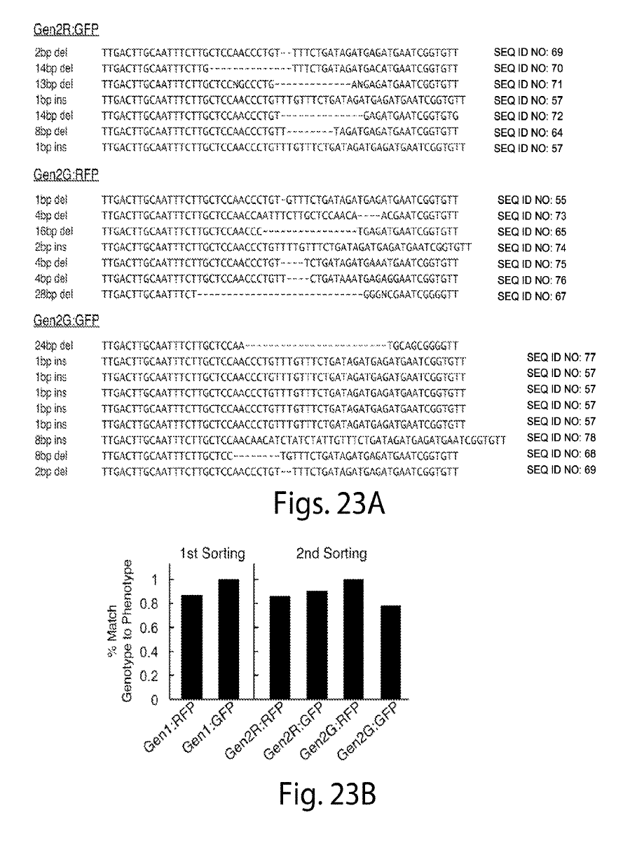

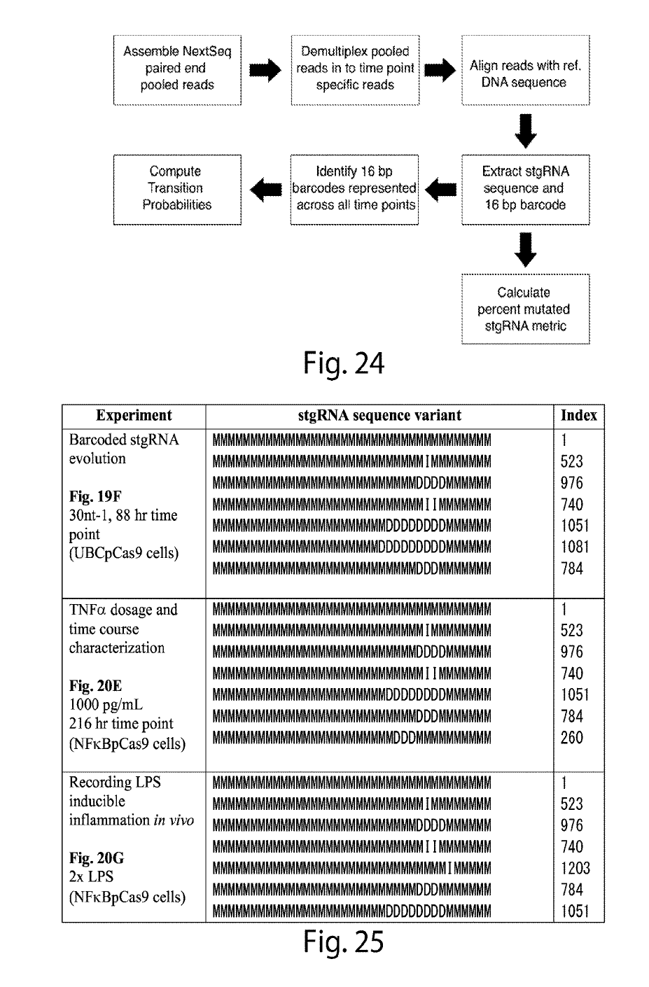

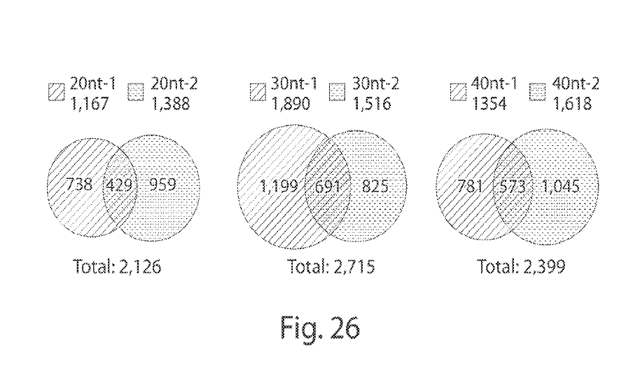

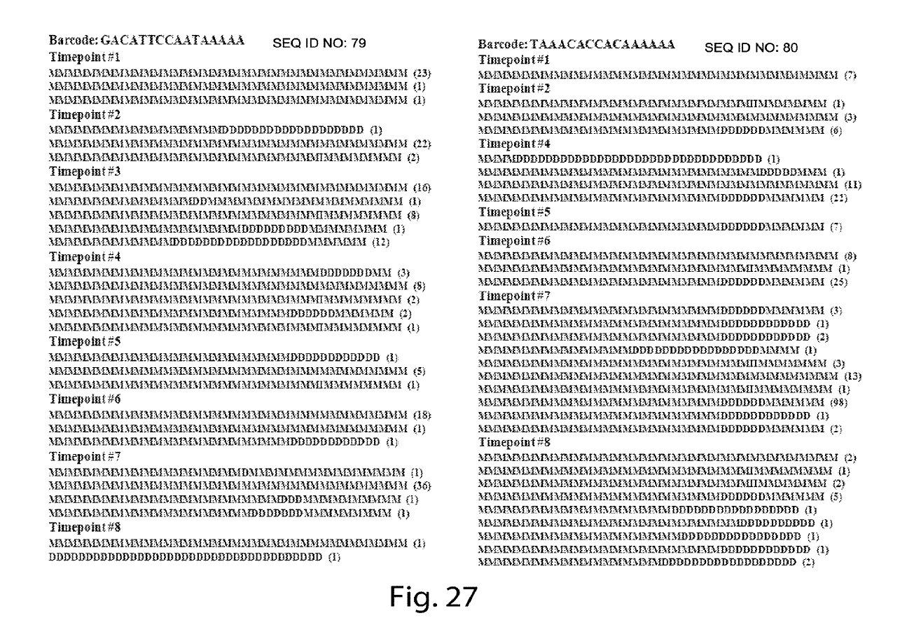

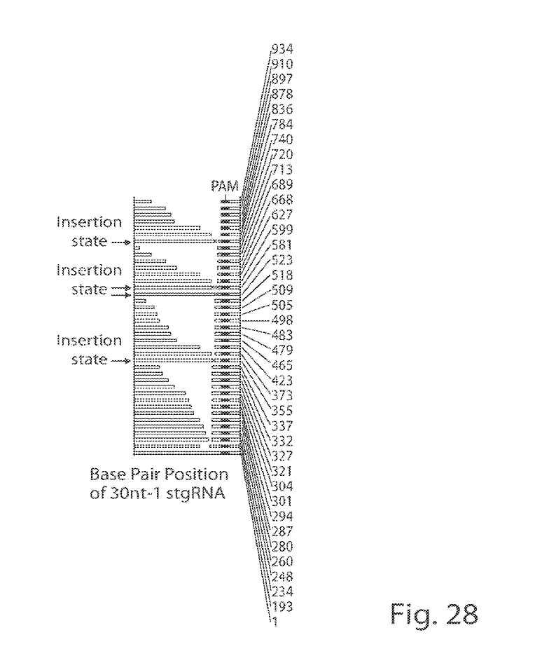

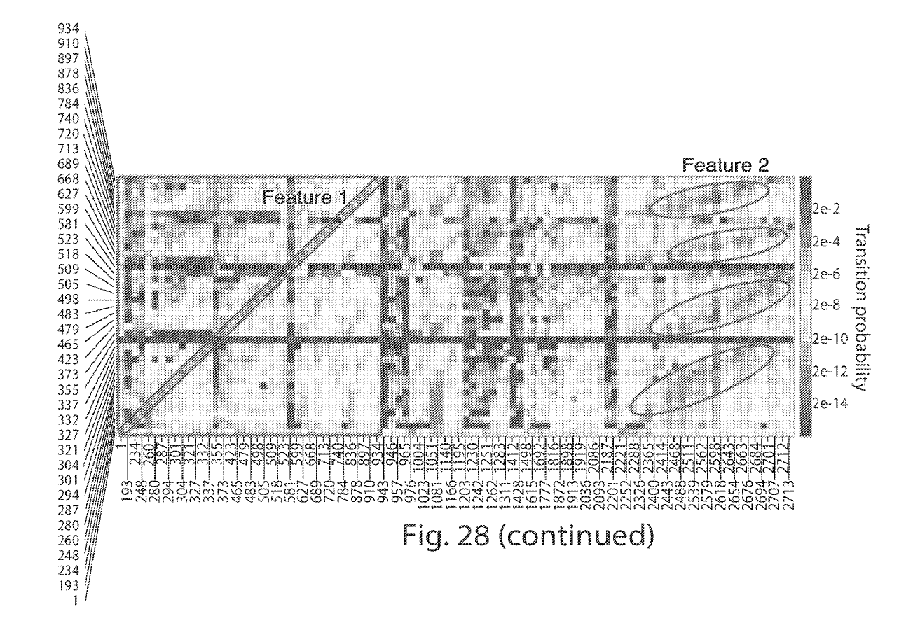

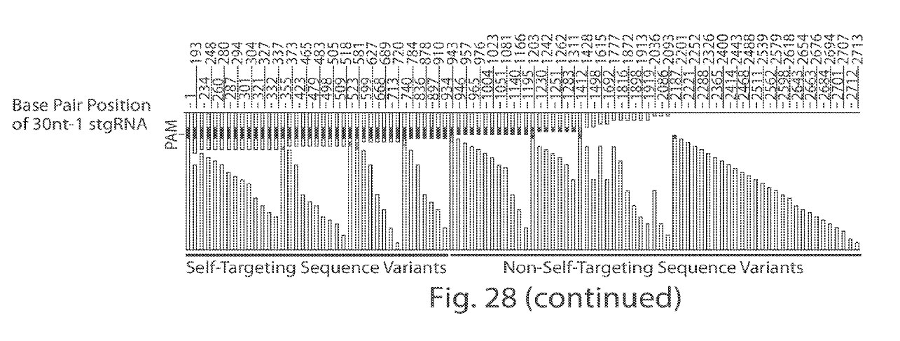

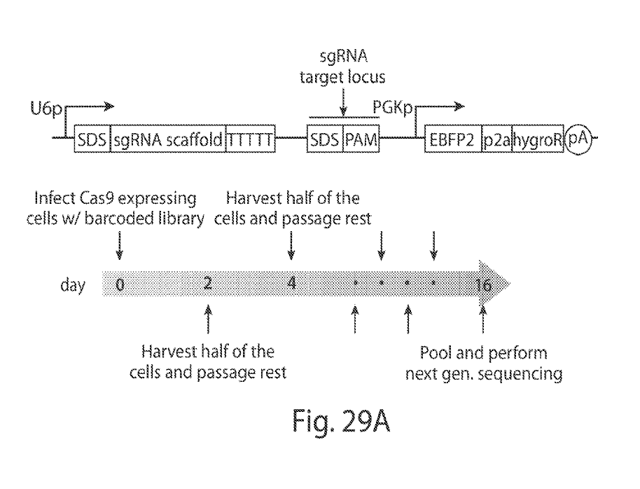

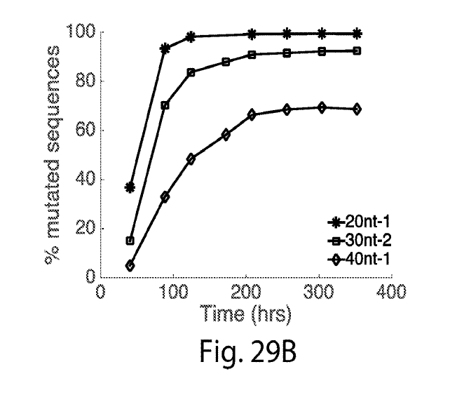

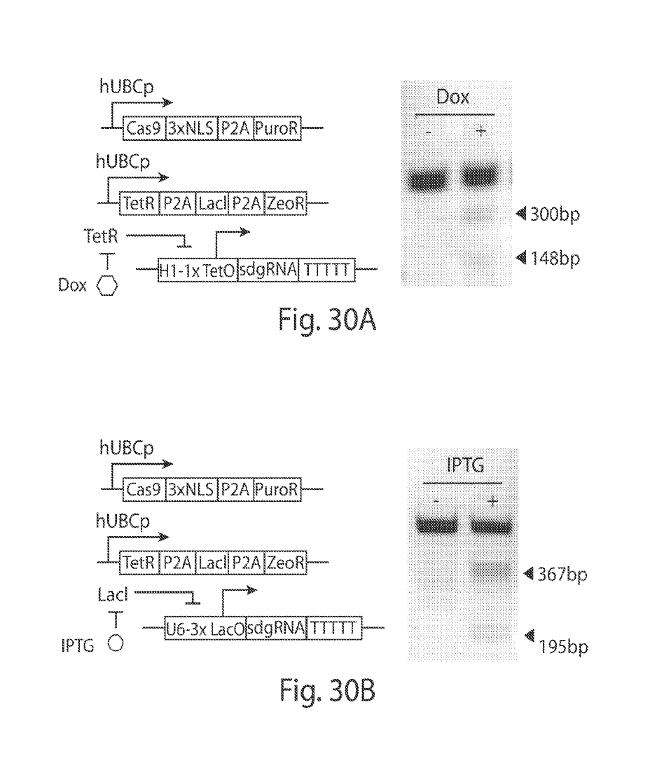

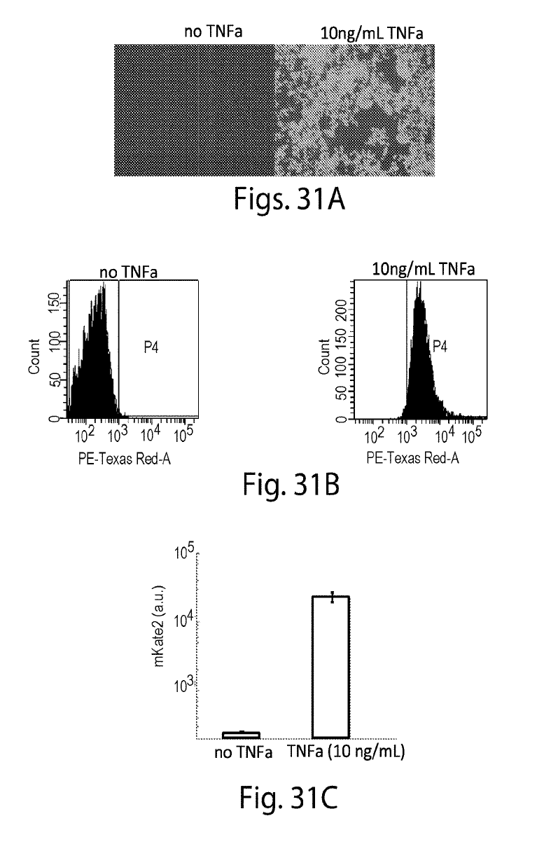

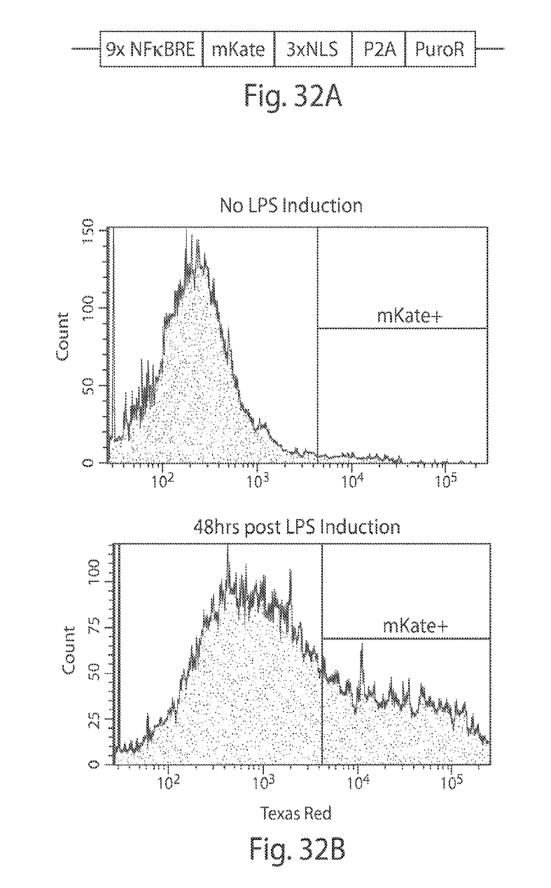

The MBTR system was subsequently used to assess changes in fluorescent gene expression within cells expressing Cas9 to track repeated mutagenesis at the stgRNA locus over time. A self-targeting construct containing a computationally designed 27 nt stgRNA driven by a modified U6 promoter was built and embedded in the MDR (construct 13, SEQ ID NO: 20, Table 2). As a control, a non-self-targeting MBTR construct with a regular sgRNA that targets a DNA sequence was built and embedded in the MDR (construct 16, SEQ ID NO: 23, Table 2). The stgRNA or control sgRNA MBTR construct (via lentiviral transduction at ˜0.3 MOI) was integrated into the genome of clonally derived Cas9-expressing HEK293T cells (hereafter called UBCp-Cas9 cells). And the cells were analyzed by two rounds of FACS sorting based on RFP and GFP levels (FIG. 18B). In both cases, we found that ˜1-5% of the cells were RFP+/GFP− or RFP−/GFP+ which were sorted into Gen1:RFP and Gen1:GFP populations, respectively) (FIGS. 18C, 18D) and <0.3% cells expressed both GFP and RFP. The Gen1:RFP and Gen1:GFP cells were cultured for 7 days, resulting in Gen2R and Gen2G populations, respectively. The Gen2R and Gen2G populations were then subjected to a 2nd round of FACS sorting. For cells with the stgRNA MBTR, a subpopulation of Gen2R cells toggled into being GFP positive, and a subpopulation of Gen2G cells toggled into being RFP positive. In contrast, cells containing the non-self-targeting sgRNA MBTR did not exhibit significant toggling of Gen1R cells into GFP positive ones, or Gen1G cells into RFP positive ones (FIGS. 18C, 18D). The toggling of fluorescent outputs observed in UBCp-Cas9 cells transduced with the stgRNA MBTR suggests that repeated nuclease cleavage at the stgRNA locus occurred within single cells. To further corroborate this finding, the stgRNA locus in individual cells from post-sorted populations in both rounds were sequenced by cloning PCR amplicons into E. coli and performing Sanger sequencing on individual bacterial colonies (FIGS. 18E and 23A-23B). We found strong correlations (75%-100% accuracy) between the sequenced genotype and observed fluorescent phenotype in all of the sorted cell populations (FIGS. 18E and 23A-23B). Together, these results confirm that repetitive mutagenesis can occur at the stgRNA locus within single cells.

### Example 14

Having established that stgRNA loci are capable of undergoing multiple rounds of targeted mutagenesis, their sequence evolution patterns over time was delineated. The characteristic properties associated with stgRNA sequence evolution may be inferred by simultaneously investigating many independently evolving genomic loci, all of which contain an exactly identical stgRNA sequence to start with (FIG. 19C). Barcoded plasmid DNA libraries were synthesized, in which the stgRNA sequence was maintained constant while a chemically randomized 16 bp barcode was placed immediately downstream of the stgRNA (FIG. 19A). Six separate DNA libraries were synthesized with stgRNAs with six unique SDSes of different lengths: 20nt-1, 20nt-2, 30nt-1, 30nt-2, 40nt-1, or 40nt-2 (constructs 19-24, SEQ ID NOs: 26-30, Table 2). A constitutively expressed EBFP2 was used as an infection marker to ensure a multiplicity of infection (MOI) of ˜0.3.

On day 0, lentiviral particles encoding each of the six stgRNA libraries were used to infect 200,000 UBCp-Cas9 cells in six separate wells of a 24 well plate. At a target MOI of ˜0.3, the infections resulted in ˜60,000 successfully transduced cells per well. For each stgRNA library, eight cell samples were collected at time points approximately spaced 48 hours apart until day 16 (FIG. 19B). All samples from eight different time points across the six different libraries were pooled together and sequenced via Illumina NextSeq. After aligning the next-generation sequencing reads to reference DNA sequences (methods), 16 bp barcodes that were observed across all the time points and the corresponding upstream stgRNA sequences were identified (FIGS. 24, 27). For each of the stgRNA libraries, it was found that >104 unique 16 bp barcoded loci that were observed across all of the eight time points (Table 1). The aligned stgRNA sequence variants were represented with words composed of a four-letter alphabet (at each bp position, the stgRNA sequence is represented by one of the letters M, I, X or D which stand for match, insertion, mismatch, deletion respectively, FIG. 25). Over 1000 unique sequence variants that were observed in any of the time points and any of the barcoded loci for each stgRNA were identified (FIG. 26). Although some sequence variants are found in common across the stgRNAs, majority of the sequence variants are unique to each stgRNA.

In FIG. 19D, the number of barcoded loci associated with each unique sequence variant derived from the original 30nt-1 stgRNA for three different time points were plotted. Although the majority of the barcoded loci corresponded to the original un-mutated stgRNA sequence for all three time points, a sequence variant containing an insertion at bp 29 and another sequence variant containing insertions at bps 29 and 30 gained significant representation by day 14. Most of the barcoded stgRNA loci evolved into just a few major sequence variants and thus these specific sequences were likely to dominate across different experimental conditions. In FIG. 25, the top seven most abundant sequence variants of the 30nt-1 stgRNA observed in three different experiments discussed in this disclosure were presented. The three experiments were performed either in vitro or in vivo with the 30nt-1 stgRNA encoded in different HEK293T-derived cell lines (UBCp-Cas9 cells) or cells in which Cas9 was regulated by the NFkappaB responsive promoter from FIGS. 19F, 20E and 20G, respectively. Six sequence variants were represented in the top seven sequence variants for all three different experiments we performed with the 30nt-1 stgRNA. Thus, stgRNA activity can result in very specific and consistent mutations.

Given the observation that stgRNAs may have characteristic sequence evolution patterns, the likelihood of an stgRNA locus transitioning from any given sequence variant to another variant due to self-targeted mutagenesis was investigated. Such likelihood was computed in the form of a transition probability matrix, which captures the probability of a sequence variant transitioning to any sequence variant within a time point (FIG. 19E). Briefly, in computing the transition probability matrix, for every sequence variant observed in a future time point (daughter), a sequence variant from the immediately preceding time point is chosen as a likely parent based on a minimal hamming distance metric. Such parent-daughter associations were computed and normalized across all time points and barcodes to result in the transition probability matrix. Since it was assumed that only stgRNA sequence variants that contain an intact PAM can self-target, transition probabilities only for states that can be self-targeting were presented. In FIG. 19E, it was found that self-targeting sequence variants are generally more likely to remain unchanged than mutagenizing within a time point (2 days), as indicated by high probabilities along the diagonal (also see FIG. 28). In addition, transition probability values are typically higher for sequence transitions below the diagonal versus for those above the diagonal, implying that sequence variants tend to progressively gain deletions. Moreover, when compared with deletion(s) containing sequence variants, insertion(s) containing sequence variants tend to have a very narrow range of sequence variants they are likely to mutagenize in to. Finally, it was noticed that prior mutated self-targeting sequence variants predominantly mutagenize in to non-self targeting sequence variants by mutagenic activities wherein the SDS encoding region remains intact but the PAM containing region is mutagenized (also see FIG. 28).

### Example 15

Having analyzed the sequence evolution characteristics of stgRNAs, a metric was computed based on the relative abundance of stgRNA sequence variants as a measure of stgRNA activity. Such a metric would enable the use of stgRNAs as intracellular recording devices in a population to store biologically relevant, time-dependent information that could be reliably interpreted after events were recorded. From the analysis of stgRNA sequence evolution, novel self-targeting sequence variants at a given time point should have arisen from prior self-targeting sequence variants and not from non-self-targeting sequence variants. Thus, the percentage of sequences that contain mutations only in the SDS-encoding region amongst all the sequences that contain an intact PAM was calculated and was designated the % mutated stgRNA metric. Such metric can serve as an indicator of stgRNA activity. In FIG. 19F, the % mutated stgRNA metric was plotted as a function of time for the six different stgRNAs. Except for the 20nt-2 stgRNA, which saturated to ˜100% by 10 days, non-saturating and reasonably linear responses of the metric for all stgRNAs over the entire 16-day experimentation period was observed. Based on the rate of increase of the % stgRNA metric (% s mutated stgRNA/time), stgRNAs encoding SDSes of longer length might have a greater capacity to maintain a linear increase in the recording metric for longer durations of time and hence are more suitable for longer-term recording applications.

A time course experiment with regular sgRNAs targeting a DNA target sequence to test their ability to serve as memory registers was also conducted (FIGS. 29A-29B). SgRNAs encoding the same 20nt-1, 30nt-2 and 40nt-1 SDSes were tested in FIG. 19F (constructs 25-27, SEQ ID NOs: 32-34 Table 2) and it was found that unlike stgRNA loci, sgRNA target loci quickly saturate the % mutated stgRNA metric at values less than 100% and do not exhibit a significant linear range.

### Example 16

StgRNA loci were placed under the control of small-molecule inducers to record chemical inputs into genomic memory registers. Soxycycline-inducible and isopropyl-β-D-thiogalactoside (IPTG)-inducible RNAP III promoters to express stgRNAs were designed, similar to previous work with shRNAs (FIG. 20A). The RNAP III H1 promoter was engineered to contain a Tet-operator, allowing for tight repression of promoter activity in the presence of the TetR protein, which can be rapidly and efficiently relieved by the addition of doxycycline (construct 29, SEQ ID NO: 36, Table 2). Similarly, An IPTG-inducible stgRNA locus was built by introducing three LacO sites into the RNAP III U6 promoter so that Lad can repress transcription of the stgRNA, which is relieved by the addition of IPTG (construct 30, SEQ ID NO: 37, Table 2). The doxycycline and IPTG-inducible stgRNAs were verified to work independently when integrated in to the genome of cells UBCp-Cas9 cells also expressing TetR and Lad (construct 28, SEQ ID NO: 35, Table 2) (FIGS. 30A-30B). Next, the doxycyline and IPTG-inducible stgRNA loci were placed on to a single lentiviral backbone (FIG. 20A, construct 31, SEQ ID NO: 38, Table 2) and integrated them into the genome of UBCp-Cas9 cells that also expressed TetR and LacI. The induction of stgRNA expression by doxycycline or IPTG led to efficient self-targeting mutagenesis at the cognate loci as detected by the T7 endonuclease I assay, while cells without exposure to doxycycline or IPTG did not (FIG. 20B). Moreover, when cells were exposed to both doxycycline and IPTG, we detected simultaneous mutation acquisition at both the loci demonstrating inducible and multiplexed molecular recording.

### Example 17

Next, stgRNA memory units that record signaling events in cells within live animals were built. A well-established acute inflammation model involving repetitive intraperitoneal (i.p.) injection of lipopolysaccharide (LPS) in mice was adapted. The activation of the NF-κB pathway plays an important role in coordinating responses to inflammation In conditions of inflammation induced by LPS, cells that sense LPS release tumor necrosis factor alpha (TNF-α which is a potent activator of the NF-κB pathway. To sense activation of the NF-κB pathway, a construct containing an NF-κB responsive promoter driving the expression of the red fluorescent protein mKate was built and stably integrated in to HEK293T cells. A >50-fold difference in expression levels when these cells were exposed to TNF-α in vitro was observed (FIGS. 31A-31C). Next, these cells were implanted into the flank of immunodeficient nude mice. After implanted cells reached a palpable volume, i.p. injection of LPS was performed and significant mKate expression (FIGS. 32A-32B) and elevated TNF-α concentrations in the serum 48 hours post LPS injection were observed (FIG. 33).

A clonal HEK293T cell line was built with an NF-κB-inducible Cas9 expression cassette and infected the cells with lentiviral particles encoding the 30nt-1 stgRNA at ˜0.3 MOI. These cells (hereafter referred to as inflammation-recording cells) accumulated stgRNA mutations, as detected with the T7 Endonuclease I assay, when induced with TNF-α (FIG. 20D). The stgRNA memory unit in inflammation-recording cells was characterized by varying the concentration (within patho-physiologically relevant concentrations and duration of exposure to TNF-α in vitro and measuring the % mutated stgRNA metric (FIG. 20E). Graded increases in the % mutated stgRNA metric as a function of time was observed, thus demonstrating that stgRNA-based memory can record temporal information on signaling events in human cells. Furthermore, higher TNF-α concentrations resulted in cells that had higher values for the % mutated stgRNA metric, indicating that signal magnitude can modulate the memory register.

### Example 18

After characterizing the in vitro time and dosage sensitivity of our inflammation recording cells, they were implanted in to mice. The implanted mice were split in to three cohorts: four mice that received no LPS injection over 13 days, four mice that received an LPS injection on day 7, and four mice that received an LPS injection on day 7 followed by another LPS injection on day 10 (FIG. 20F). The genomic DNA of implanted cells was extracted from all cohorts on day 13 and the 30nt-1 stgRNA locus was PCR amplified and sequenced via next-generation sequencing. A direct correlation between the LPS dosage and the % mutated stgRNA metric was observed, with increasing numbers of LPS injections resulting in increased % mutated stgRNA (FIG. 20G). The results indicate that stgRNA memory registers can be used in vivo to record physiologically relevant biological signals

In FIGS. 19E and 20F, PCR was used to amplify the stgRNA loci from ˜30,000 cells and then calculated the % mutated stgRNA metric as a readout of genomic memory. However, access to tissues or biological samples could be limited in certain in vivo contexts. To investigate the sensitivity of our stgRNA-encoded memory when the input biological material is restricted, 1:100 dilutions of the genomic DNA extracted from the TNFα-treated inflammation-recording cells in FIG. 4E were sampled, which corresponds to ˜300 cells, in triplicate followed by PCR amplification, sequencing, and calculation of the % mutated stgRNA metric (FIG. 34). Very little deviation were found between the % mutated sgRNA metric between samples with ˜300 cells versus those from ˜30,000 cells. The tight correspondence may be due to stgRNA evolution towards very few, dominating sequence variants, as was observed in FIGS. 19D and 25.

Provided herein are architectures for self-targeting guide RNAs (stgRNAs) that can repeatedly direct Cas9 activity against the DNA loci that encode the stgRNAs. This technology enables the creation of self-contained genomic memory units in human cell populations. stgRNAs can be engineered by introducing a PAM into the sgRNA sequence, and mutations accumulate repeatedly in stgRNA-encoding loci over time with the MBTR system. Furthermore, a computational metric that can be used to map the extent of stgRNA mutagenesis in a cell population to the duration or magnitude of the recorded input signal is provided. Results demonstrate that percent mutated stgRNAs increases with the magnitude and duration of input signals, thus resulting in long-lasting analog memory stored in the genomic DNA of human cell populations. Because the stgRNA loci can be multiplexed for memory storage and function in vivo, this approach for analog memory in human cells can used to map dynamical and combinatorial sets of gene regulatory events without the need for continuous cell imaging or destructive sampling. For example, cellular records can be used to monitor the spatiotemporal heterogeneity of molecular stimuli that cancer cells are exposed to within tumor microenvironments, such as exposure to hypoxia, pro-inflammatory cytokines, and other soluble factors. One can also track the extent to which specific signaling pathways are activated during disease progression or development, such as the mitogen-activated protein kinase (MAPK), Wnt, Sonic Hedehog (SHH), TGF-α regulated signaling pathways in normal development and disease.

To enhance the controllability of mutations that arise over time, small molecule inhibitors of the components of aNHEJ, including ligase III and PARP1, respectively, may be used. Engineering and characterizing a larger library of stgRNA sequences may help to identify additional efficient memory registers.

### Methods

Plasmids

The Cas9 expressing plasmid CMVp-Cas9-3xNLS was built by PCR extension of 3x SV40 Nuclear Localization Signal (NLS) to the 3′ end of S. pyogenes Cas9 amplified from LentiCRISPRv1 (Addgene #49535). The resulting Cas9-3xNLS amplicon was cloned in to the SacI/XmaI digested CMVp-HHRibo-gRNA1-HDVRibopA (Construct 15, Nissim L, et al. 2014) plasmid via Gibson assembly.

The gRNA expression plasmid containing pPGK1-eBFP2 described in (Nissim L, et al. 2014) was modified to contain a p2a-linked hygromycin resistance gene (hygroR) to build the plasmid U6p-gRNA-pPGK1-EBFP2-p2a-hygroR. Different stgRNAs were engineered in to the SacI/XbaI digested U6p-gRNA-pPGK1-EBFP2-p2a-hygroR plasmid via Gibson assembly. The gRNA derived plasmids were then cloned in to the PacI/EcoRI digested 3rd generation lentiviral plasmid FUGw (Addgene #14883) via Gibson assembly.

Reverse-Tet-transactivator (rTta3) and pTRE was amplified from Tet-On plasmid systems (Clontech, Ltd). rTta3, along with p2a-linked Zeocin resistance gene (zeoR) were cloned in to BamHI/EcoRI digested FUGw via Gibson Assembly to build hUBCp-rtTA3-p2a-ZeoR.

pTRE was cloned with mKate2 (Evrogen) and p2a-linked puromycin resistance gene (puroR) via Gibson assembly in to PacI/EcoRI digested FUGw to build pTRE-mKate2-puroR.

9xNF-κBRE containing 9 copies of the NF-κB response element (RE) was synthesized by Integrated DNA Technologies (IDT). 9xNF-κBRE, minimal MLP promoter, mKate2 (Evrogen) and p2a-linked puromycin resistance gene were cloned via Gibson assembly in to PacI/EcoRI digested FUGw to build 9xNF-κBREp-mKate2-puroR.

Cell Lines

Stable cell lines expressing the wild-type and various modified stgRNAs (mod1 through mod5) were built by lentiviral transduction of HEK293T cells followed by selection with hygromycin. LV particles were produced by transfecting 200,000 HEK293T cells with 1 μg of lentiviral backbone containing plasmid 0.5 μg of pCMV-VSV-G (Addgene #8454) and 0.5 μg of pCMV-dR8.2 (Addgene #8455). The cell culture supernatant containing LV particles was collected 48 hrs post transfection, filtered with a 0.2 mM Cellulose acetate filter and was used to infect HEK293T cells supplemented with 8 mg/mL polybrene. Successfully transduced cells were obtained by selection with hygromycin at 300 μg/mL for four days.

Stable cell lines expressing rTta3 (reverse tetracycline inducible transactivator) were built by lentiviral transduction of HEK293T cells followed by selection with Zeocin at 100 ug/mL for four days. LV particle production and transduction was as described above. After subsequent transduction of the rTta3 expressing cell line with LVs encoding pTRE-mKate2-puroR, cells were induced with 1 μg/mL doxycycline for a day and selected with 3 μg/mL puromycin for four days to build a stable Dox inducible cell line expressing Cas9.

Similarly HEK 293T cells transduced with LVs encoding 9xNF-κBREp-Cas9-puroR were induced with 50 ng/mL TNFα for a day and selected with 3 μg/mL puromycin for four days to build a stable, TNFα inducible cell line expressing Cas9.

Experimental Design and Assays

Once stable cell lines containing different variants of the stgRNAs have been built, they were transfected in six-well plates with CMVp-Cas9-3xNLS or a plasmid expressing mYFP. After 96 hours of incubation at 37° C., genomic DNA was extracted using the QuickExtract DNA Extraction solution (Epicentre). Genomic PCRs were performed in 50 μL reactions with the following primers

JP1710-GCAGAGATCCAGTTTGGGGGGTTCCGCGCAC (SEQ ID NO:6) and

JP1711-CCCGGTAGAATTCCTCGACGTCTAATGCCAAC (SEQ ID NO:7) at 65° C. 30s, 25s/Cycle extension at 72° C., 29 cycles. Purified PCR DNA was then used in T7 Endonuclease I (T7E1) assays. 400 ng of per DNA was used per 20 μL T7E1 reaction mixture (NEB Protocols, M0302).

The targeting efficiency in FIG. 7 was calculated by estimating the fraction of DNA cleaved by quantifying the image intensity of the SYBR-stained DNA gels. The values reported as targeting efficiency were computed as

%=100×(1−(1−fraction cleaved))̂(½)

For time course experiment in FIG. 10 and FIG. 11, a master transfection of either CMVp-Cas9-3xNLS or a plasmid expressing mYFP was performed on stable cell lines expressing stgRNA or wild-type gRNA with 20 nt SDS. 200,000 cell aliquots were then plated in to separate wells of a six well plate to be assayed at different time points as illustrated in FIG. 9.

Genomic DNA was extracted from cells using QuickExtract. Barcoded PCRs were pooled and sequenced on the MIT BioMicroCenter (MIT BMC) MiSeq platform. Sequencing reads were processed using a custom written C/C++ code and were aligned to the reference stgRNA sequence using a custom written implementation of the Needleman-Wunsch algorithm. After sequences have been aligned the percentage of indels and point mutations was calculated in Matlab and plotted in FIG. 10 and FIG. 11.

T7 Endonuclease I (T7 E1) Assays and Sanger Sequencing

Genomic DNA from respective cell lines containing the sagRNA or the sgRNA loci was extracted using the QuickExtract DNA extraction solution (Epicentre). Genomic pers were performed using the KAPA-HiFi polymerase (KAPA biosystems) using the primers JP1710-GCAGAGATCCAGTTTGGGGGGTTCCGCGCAC (SEQ ID NO: 6) and JP1711-CCCGGTAGAATTCCTCGACGTCTAATGCCAAC (SEQ ID NO: 7) at 65° C. 30s, 25s/Cycle extension at 72 C, 29 cycles. Purified per DNA was then used in T7 Endonuclease I (T7E1) assays. Specifically, 400 ng of per DNA was used per 20 uL T7E1 reaction mixture (NEB Protocols, M0302). The hybridization protocol used for per DNA in T7E1 assays is indicated in the Table 1. For Sanger sequencing, PCR products from mutated genomic DNA were cloned in to the KpnI/NheI sites of construct 13 and transformed in to E. Coli (DH5a, NEB). Single colonies of bacteria were sequenced using the RCA method (Genewiz, Inc).

Cell Culture, Transfections and Lentiviral Infections

Cell culture and transfections were done as described earlier. Lentiviruses were packaged using the FUGw backbone (Addgene #25870) in HEK-293T cells. Filtered lentiviruses were used to infect respective cell lines in the presence of polybrene (8 ug/mL). Successful lentiviral integration was confirmed by using lentiviral plasmid constructs constitutively expressing fluorescent proteins to serve as infection markers.

Clonal Cell Lines and DNA Constructs

A lentiviral plasmid construct expressing spCas9, codon optimized for expression in human cells fused to the puromycin resistance with a p2a linker was built from the taCas9 plasmid (construct 12, SEQ ID NO: 19, Table 2). The UBCp-Cas9 cell line was constructed by infecting early passage HEK-293T cells (ATCC CRL-11268) with high titre lentiviral particles encoding the above plasmid and selecting for clonal populations grown in the presence of puromycin (7 ug/mL). The inflammation recording cell line was built by infecting HEK-293T cells with higher titer lentiviral particles encoding NFκB responsive Cas9 expressing construct (construct 33, SEQ ID NO: 40, Table 2). Transduced cells were induced with 1 ng/mL TNF-α for three days followed by selection with 3 ug/mL puromycin. Inflammation recording cells were then clonally isolated in the absence of TNF-α Cell lines used to test stgRNA activity were built by infecting HEK293T cells with lentiviral particles encoding constructs 1 through 6 (SEQ ID NOs: 8-13, Table 2) and selecting for successfully transduced cells with 300 ug/mL hygromycin.

Flow Cytometry, Microscopy and Sanger Sequencing

Before analysis and sorting, cells were with PBS and re-suspended in PBS+2% FBS. Cells were sorted using Beckmann Coulter MoFlo cell sorter at MIT Koch Institute's flow Cytometry core. Flow cytometry analysis was performed with Becton Dickinson LSRFortessa. Fluorescent microscopic images of cells were produced by Thermo Scientific's EVOS cell imager. The cells were directly imaged from tissue culture plates.

Next Generation Sequencing and Alignment

Genomic DNA from respective cell lines was extracted using QuickExtract (Epicenter) and amplified using sequence specific primers containing Illumina adapter sequences P5-AATGATACGGCGACCACCGAGATCTACAC (SEQ ID NO: 41) and P7-CAAGCAGAAGACGGCATACGAGAT (SEQ ID NO: 42) as primer overhangs. Multiple PCR samples were multiplexed together and sequenced on a single flow cell using 8 bp multiplexing barcodes incorporated via reverse primers. The barcode library stgRNA samples in FIGS. 19A-19F were sequenced on the NextSeq platform while the 20nt-1 stgRNA samples in FIGS. 17A-17E, the regular sgRNA samples in FIG. 28, the mouse tumor PCR samples in FIG. 20G were sequenced on the MiSeq platform. Paired end reads were assembled using the PEAR package. Optimal sequence alignment was performed by a custom written C++ code implementing the SS-2 algorithm using affine gap costs with a gap opening penalty of 2.5 and a gap continuation penalty of 0.5. The aligned sequences were represented using a four-letter alphabet in the ‘MIXD’ format where M represents a match, I represents an insertion, X represents a mismatch and D represents a deletion. At each base-pair position, the sequence aligned base pair is represented by one of the following letters: ‘M’, ‘I’, ‘X’ or ‘D’—representing a match, insertion, mismatch or a deletion respectively (FIG. 25).

Barcoded stgRNA Sequence Evolution and Transition Probabilities

As a first step, barcode vs. aligned stgRNA sequence (in the ‘MIXD’ format) associations were built by aligning each individual NextSeq read to the reference DNA sequence. Only the 16 bp barcodes that were represented in all of the time points were considered for further analysis. To compute the transition probabilities, barcode and stgRNA sequence variant associations that were generated for each time point (FIG. 27) were used. Every possible two-wise combination of sequence variants associated with the same barcoded locus but consecutive time points were evaluated for a parent-daughter association. For every sequence variant in a future time point (a daughter), a sequence variant from amongst all of the sequence variants in the immediately preceding time point that has the minimum hamming distance to the daughter sequence variant was assigned a parent. Since the presence of an intact PAM is an absolute requirement for the self-targeting capability of stgRNAs, only the sequence variants that contained an intact PAM were considered as potential parents. Many parent-daughter associations were computed across all the barcodes and time points resulting in a frequency score for each parent-daughter association. Finally, the frequencies were normalized to sum to one to result in a probability transition matrix.

Design of Longer stgRNAs

Longer stgRNAs were designed using the ViennaRNA package. Specifically, the RNAfold software there-in was used to generate SDSes that retain the native structure of the guide RNA handle and no secondary structures in the SDS encoding region as the minimum free energy structure.

In Vivo Inflammation Model

Female BALB/c-nu/+ mice were obtained from the rodent breeding colony at Charles River Laboratory. They were specific pathogen free and maintained on sterilized water and animal food. Engineered HEK293T cells were suspended in matrigel (Corning, N.Y.) in 1:1 ratio with cell growth medium. 2×106 cells were implanted subcutaneously at the flank region of the mice. Where indicated, mice were injected intraperitoneally with LPS (from Escherichia coli serotype 0111:B4, prepared by from sterile ready-made solution) (Sigma Chemical Co., St. Louis, Mo.) dissolved in 0.1 ml PBS.

TABLE 1
Number of 16 bp barcodes represented across
all the time points for each stgRNA

| Plasmid library | Number of unique 16 bp barcodes |
|---|---|
| 20nt-1 | 18,675 |
| 20nt-2 | 25,876 |
| 30nt-1 | 44,457 |
| 30nt-2 | 14,408 |
| 40nt-1 | 21,027 |
| 40nt-2 | 16,506 |

TABLE 2
List of DNA constructs used in this study

| Construct name | DNA sequence |
|---|---|
| Construct 1- | TGTACAAAAAAGCAGGCTTTAAAGGAACCAATTCAGTCGACTGGATCCGGTACCAAG |
| U6p_20nt1_wt_sgRNA | GTCGGGCAGGAAGAGGGCCTATTTCCCATGATTCCTTCATATTTGCATATACGATACA |
| F | AGGCTGTTAGAGAGATAATTAGAATTAATTTGACTGTAAACACAAAGATATTAGTAC |
| SEQ ID NO: 8 | AAAATACGTGACGTAGAAAGTAATAATTTCTTGGGTAGTTTGCAGTTTTAAAATTATG |
| | TTTTAAAATGGACTATCATATGCTTACCGTAACTTGAAAGTATTTCGATTTCTTGGCTT |
| | TATATATCTTGTGGAAAGGACGGAACACCGTAAGTCGGAGTACTGTCCTGTTTTAGAG |
| | CTAGAAATAGCAAGTTAAAATAAGGCTAGTCCGTTATCAACTTGAAAAAGTGGCACC |
| | GAGTCGGTGCTTTTTTT |
| Construct 2- | TGTACAAAAAAGCAGGCTTTAAAGGAACCAATTCAGTCGACTGGATCCGGTACCAAG |
| U6p_20nt1_mod1_sgRNA | GTCGGGCAGGAAGAGGGCCTATTTCCCATGATTCCTTCATATTTGCATATACGATACA |
| SEQ ID NO: 9 | AGGCTGTTAGAGAGATAATTAGAATTAATTTGACTGTAAACACAAAGATATTAGTAC |
| | AAAATACGTGACGTAGAAAGTAATAATTTCTTGGGTAGTTTGCAGTTTTAAAATTATG |
| | TTTTAAAATGGACTATCATATGCTTACCGTAACTTGAAAGTATTTCGATTTCTTGGCTT |
| | TATATATCTTGTGGAAAGGACGAAACACCGTAAGTCGGAGTACTGTCCTGGGTTAGA |
| | GCTAGAAATAGCAAGTTAAAATAAGGCTAGTCCGTTATCAACTTGAAAAAGTGGCAC |
| | CGAGTCGGTGCTTTTTT |
| Construct 3- | TGTACAAAAAAGCAGGCTTTAAAGGAACCAATTCAGTCGACTGGATCCGGTACCAAG |
| U6p_20nt1_mod2_sgRNA | GTCGGGCAGGAAGAGGGCCTATTTCCCATGATTCCTTCATATTTGCATATACGATACA |
| (stgRNA) | AGGCTGTTAGAGAGATAATTAGAATTAATTTGACTGTAAACACAAAGATATTAGTAC |
| SEQ ID NO: 10 | AAAATACGTGACGTAGAAAGTAATAATTTCTTGGGTAGTTTGCAGTTTTAAAATTATG |
| | TTTTAAAATGGACTATCATATGCTTACCGTAACTTGAAAGTATTTCGATTTCTTGGCTT |
| | TATATATCTTGTGGAAAGGACGAAACACCGTAAGTCGGAGTACTGTCCTGGGTTAGA |
| | GCTAGAAATAGCAAGTTAACCTAAGGCTAGTCCGTTATCAACTTGAAAAAGTGGCAC |
| | CGAGTCGGTGCTTTTTT |
| Construct 4- | TGTACAAAAAAGCAGGCTTTAAAGGAACCAATTCAGTCGACTGGATCCGGTACCAAG |
| U6p_20nt1_mod3_sgRNA | GTCGGGCAGGAAGAGGGCCTATTTCCCATGATTCCTTCATATTTGCATATACGATACA |
| SEQ ID NO: 11 | AGGCTGTTAGAGAGATAATTAGAATTAATTTGACTGTAAACACAAAGATATTAGTAC |
| | AAAATACGTGACGTAGAAAGTAATAATTTCTTGGGTAGTTTGCAGTTTTAAAATTATG |
| | TTTTAAAATGGACTATCATATGCTTACCGTAACTTGAAAGTATTTCGATTTCTTGGCTT |
| | TATATATCTTGTGGAAAGGACGAAACACCGTAAGTCGGAGTACTGTCCTCGGTTAGAG |
| | CTAGAAATAGCAAGTTAACCGAAGGCTAGTCCGTTATCAACTTGAAAAAGTGGCACC |
| | GAGTCGGTGCTTTTTT |
| Construct 5- | TGTACAAAAAAGCAGGCTTTAAAGGAACCAATTCAGTCGACTGGATCCGGTACCAAG |
| U6p_20nt1_mod4_sgRNA | GTCGGGCAGGAAGAGGGCCTATTTCCCATGATTCCTTCATATTTGCATATACGATACA |
| SEQ ID NO: 12 | AGGCTGTTAGAGAGATAATTAGAATTAATTTGACTGTAAACACAAAGATATTAGTAC |
| | AAAATACGTGACGTAGAAAGTAATAATTTCTTGGGTAGTTTGCAGTTTTAAAATTATG |
| | TTTTAAAATGGACTATCATATGCTTACCGTAACTTGAAAGTATTTCGATTTCTTGGCTT |
| | TATATATCTTGTGGAAAGGACGAAACACCGTAAGTCGGAGTACTGTCCTCGGTTTTAG |
| | AGCTAGAAATAGCAAGTTAAAATAAGGCTAGTCCGTTATCAACTTGAAAAAGTGGCA |
| | CCGAGTCGGTGCTTTTTT |
| Construct 6- | TGTACAAAAAAGCAGGCTTTAAAGGAACCAATTCAGTCGACTGGATCCGGTACCAAG |
| U6p_20nt1_mod5_sgRNA | GTCGGGCAGGAAGAGGGCCTATTTCCCATGATTCCTTCATATTTGCATATACGATACA |
| SEQ ID NO: 13 | AGGCTGTTAGAGAGATAATTAGAATTAATTTGACTGTAAACACAAAGATATTAGTAC |
| | AAAATACGTGACGTAGAAAGTAATAATTTCTTGGGTAGTTTGCAGTTTTAAAATTATG |
| | TTTTAAAATGGACTATCATATGCTTACCGTAACTTGAAAGTATTTCGATTTCTTGGCTT |
| | TATATATCTTGTGGAAAGGACGAAACACCGTAAGTCGGAGTACTGTCCTGGGTTTTAG |
| | AGCTAGAAATAGCAAGTTAAAATAAGGCTAGTCCGTTATCAACTTGAAAAAGTGGCA |
| | CCGAGTCGGTGCTTTTTT |
| Construct 7- | TAGTTATTAATAGTAATCAATTACGGGGTCATTAGTTCATAGCCCATATATGGAGTTC |
| CMVp_Cas9_3xNLS_HSVpA | CGCGTTACATAACTTACGGTAAATGGCCCGCCTGGCTGACCGCCCAACGACCCCCGCC |
| SEQ ID NO: 14 | CATTGACGTCAATAATGACGTATGTTCCCATAGTAACGCCAATAGGGACTTTCCATTG |
| | ACGTCAATGGGTGGAGTATTTACGGTAAACTGCCCACTTGGCAGTACATCAAGTGTAT |
| | CATATGCCAAGTACGCCCCCTATTGACGTCAATGACGGTAAATGGCCCGCCTGGCATT |
| | ATGCCCAGTACATGACCTTATGGGACTTTCCTACTTGGCAGTACATCTACGTATTAGTC |
| | ATCGCTATTACCATGGTGATGCGGTTTTGGCAGTACATCAATGGGCGTGGATAGCGGT |
| | TTGACTCACGGGGATTTCCAAGTCTCCACCCCATTGACGTCAATGGGAGTTTGTTTTG |
| | GCACCAAAATCAACGGGACTTTCCAAAATGTCGTAACAACTCCGCCCCATTGACGCA |
| | AATGGGCGGTAGGCGTGTACGGTGGGAGGTCTATATAAGCAGAGCTGGTTTATGAAC |
| | CGTCAGATCCGAGCTCATCACCGGTGCGCTGCCACCATGGACAAGAAGTACAGCATC |
| | GGCCTGGACATCGGCACCAACTCTGTGGGCTGGGCCGTGATCACCGACGAGTACAAG |
| | GTGCCCAGCAAGAAATTCAAGGTGCTGGGCAACACCGACCGGCACAGCATCAAGAAG |
| | AACCTGATCGGAGCCCTGCTGTTCGACAGCGGCGAAACAGCCGAGGCCACCCGGCTG |
| | AAGAGAACCGCCAGAAGAAGATACACCAGACGGAAGAACCGGATCTGCTATCTGCA |
| | AGAGATCTTCAGCAACGAGATGGCCAAGGTGGACGACAGCTTCTTCCACAGACTGGA |
| | AGAGTCCTTCTTGGTGGAAGAGGATAAGAAGCACGAGCGGCACACCCCATCTTCGGCAA |
| | CATCGTGGACGAGGTGGCCTACCACGAGAAGTACCCCACCATCTACCACCTGAGAAA |
| | GAAACTGGTGGACAGCACCGACAAGGCCGACCTGCGGCTGATCTATCTGGCCCTGGC |
| | CCACATGATCAAGTTCCGGGGCCACTTCCTGATCGAGGGCGACCTGAACCCCGACAA |
| | CAGCGACGTGGACAAGCTGTTCATCCAGCTGGTGCAGACCTACAACCAGCTGTTCGA |
| | GGAAAACCCCATCAACGCCAGCGGCGTGGACGCCAAGGCCATCCTGTCTGCCAGACT |
| | GAGCAAGAGCAGACGGCTGGAAAATCTGATCGCCCAGCTGCCCGGCGAGAAGAAGA |
| | ATGGCCTGTTCGGAAACCTGATTGCCCTGAGCCTGGGCCTGACCCCCAACTTCAAGAG |
| | CAACTTCGACCTGGCCGAGGATGCCAAACTGCAGCTGAGCAAGGACACCTACGACGA |
| | CGACCTGGACAACCTGCTGGCCCAGATCGGCGACCAGTACGCCGACCTGTTTCTGGCC |
| | GCCAAGAACCTGTCCGACGCCATCCTGCTGAGCGACATCCTGAGAGTGAACACCGAG |
| | ATCACCAAGGCCCCCCTGAGCGCCTCTATGATCAAGAGATACGACGAGCACCACCAG |
| | GACCTGACCCTGCTGAAAGCTCTCGTGCGGCAGCAGCTGCCTGAGAAGTACAAAGAG |
| | ATTTTCTTCGACCAGAGCAAGAACGGCTACGCCGGCTACATTGACGGCGGAGCCAGC |
| | CAGGAAGAGTTCTACAAGTTCATCAAGCCCATCCTGGAAAAGATGGACGGCACCGAG |
| | GAACTGCTCGTGAAGCTGAACAGAGAGGACCTGCTGCGGAAGCAGCGGACCTTCGAC |
| | AACGGCAGCATGCCCCACCAGATCCACCTGGGAGAGCTGCACGCCATTCTGCGGCGG |
| | CAGGAAGATTTTTACCCATTCCTGAAGGACAACCGGGAAAAGATCGAGAAGATCCTG |
| | ACCTTCCGCATCCCCTACTACGTGGGCCCTCTGGCCAGGGGAAACAGCAGATTCGCCT |
| | GGATGACCAGAAAGAGCGAGGAAACCATCACCCCCTGGAACTTCGAGGAAGTGGTGG |
| | ACAAGGGCGCTTCCGCCCAGAGCTTCATCGAGCGGATGACCAACTTCGATAAGAACC |
| | TGCCCAACGAGAAGGTGCTGCCCAAGCACAGCCTGCTGTACGAGTACTTCACCGTGTA |
| | TAACGAGCTGACCAAAGTGAAATACGTGACCGAGGGAATGAGAAAGCCCGCCTTCCT |
| | GAGCGGCGAGCAGAAAAAGGCCATCGTGGACCTGCTGTTCAAGACCAACCGGAAAGT |
| | GACCGTGAAGCAGCTGAAAGAGGACTACTTCAAGAAAATCGAGTGCTTCGACTCCGT |
| | GGAAATCTCCGGCGTGGAAGATCGGTTCAACGCCTCCCTGGGCACATACCACGATCTG |
| | CTGAAAATTATCAAGGACAAGGACTTCCTGGACAATGAGGAAAACGAGGACATTCTG |
| | GAAGATATCGTGCTGACCCTGACACTGTTTGAGGACAGAGAGATGATCGAGGAACGG |
| | CTGAAAACCTATGCCCACCTGTTCGACGACAAAGTGATGAAGCAGCTGAAGCGGCGG |
| | AGATACACCGGCTGGGGCAGGCTGAGCCGGAAGCTGATCAACGGCATCCGGGACAAG |
| | CAGTCCGGCAAGACAATCCTGGATTTCCTGAAGTCCGACGGCTTCGCCAACAGAAACT |
| | TCATGCAGCTGATCCACGACGACAGCCTGACCTTTAAAGAGGACATCCAGAAAGCCC |
| | AGGTGTCCGGCCAGGGCGATAGCCTGCACGAGCACATTGCCAATCTGGCCGGCAGCC |
| | CCGCCATTAAGAAGGGCATCCTGCAGACAGTGAAGGTGGTGGACGAGCTCGTGAAAG |
| | TGATGGGCCGGCACAAGCCCGAGAACATCGTGATCGAAATGGCCAGAGAGAACCAG |
| | ACCACCCAGAAGGGACAGAAGAACAGCCGCGAGAGAATGAAGCGGATCGAAGAGGG |
| | CATCAAAGAGCTGGGCAGCCAGATCCTGAAAGAACACCCCGTGGAAAACACCCAGCT |
| | GCAGAACGAGAAGCTGTACCTGTACTACCTGCAGAATGGGCGGGATATGTACGTGGA |
| | CCAGGAACTGGACATCAACCGGCTGTCCGACTACGATGTGGACCATATCGTGCCTCAG |
| | AGCTTTCTGAAGGACGACTCCATCGACAACAAGGTGCTGACCAGAAGCGACAAGAAC |
| | CGGGGCAAGAGCGACAACGTGCCCTCCGAAGAGGTCGTGAAGAAGATGAAGAACTA |
| | CTGGCGGCAGCTGCTGAACGCCAAGCTGATTACCCAGAGAAAGTTCGACAATCTGAC |
| | CAAGGCCGAGAGAGGCGGCCTGAGCCAACTGGATAAGGCCGGCTTCATCAAGAGAC |
| | AGCTGGTGGAAACCCGGCAGATCACAAAGCACGTGGCACAGATCCTGGACTCCCGGA |
| | TGAACACTAAGTACGACGAGAATGACAAGCTGATCCGGGAAGTGAAAGTGATCACCC |
| | TGAAGTCCAAGCTGGTGTCCGATTTCCGGAAGGATTTCCAGTTTTACAAAGTGCGCGA |
| | GATCAACAACTACCACCACGCCCACGACGCCTACCTGAACGCCGTCGTGGGAACCGC |
| | CCTGATCAAAAAGTACCCTAAGCTGGAAAGCGAGTTCGTGTACGGCGACTACAAGGT |
| | GTACGACGTGCGGAAGATGATCGCCAAGAGCGAGCAGGAAATCGGCAAGGCTACCG |
| | CCAAGTACTTCTTCTACAGCAACATCATGAACTTTTTCAAGACCGAGATTACCCTGGC |
| | CAACGGCGAGATCCGGAAGCGGCCTCTGATCGAGACAAACGGCGAAACCGGGGAGA |
| | TCGTGTGGGATAAGGGCCGGGATTTTGCCACCGTGCGGAAAGTGCTGAGCATGCCCC |
| | AAGTGAATATCGTGAAAAAGACCGAGGTGCAGACAGGCGGCTTCAGCAAAGAGTCTA |
| | TCCTGCCCAAGAGGAACAGCGATAAGCTGATCGCCAGAAAGAAGGACTGGGACCCTA |
| | AGAAGTACGGCGGCTTCGACAGCCCCACCGTGGCCTATTCTGTGCTGGTGGTGGCCAA |
| | AGTGGAAAAGGGCAAGTCCAAGAAACTGAAGAGTGTGAAAGAGCTGCTGGGGATCA |
| | CCATCATGGAAAGAAGCAGCTTCGAGAAGAATCCCATCGACTTCTGGAAGCCAAGG |
| | GCTACAAAGAAGTGAAAAAGGACCTGATCATCAAGCTGCCTAAGTACTCCCTGTTCG |
| | AGCTGGAAAACGGCCGGAAGAGAATGCTGGCCTCTGCCGGCGAACTGCAGAAGGGA |
| | AACGAACTGGCCCTGCCCTCCAAATATGTGAACTTCCTGTACCTGGCCAGCCACTATG |
| | AGAAGCTGAAGGGCTCCCCCGAGGATAATGAGCAGAAACAGCTGTTTGTGGAACAGC |
| | ACAAGCACTACCTGGACGAGATCATCGAGCAGATCAGCGAGTTCTCCAAGAGAGTGA |
| | TCCTGGCCGACGCTAATCTGGACAAAGTGCTGTCCGCCTACAACAAGCACCGGGATA |
| | AGCCCATCAGAGAGCAGGCCGAGAATATCATCCACCTGTTTACCCTGACCAATCTGGG |
| | AGCCCCTGCCGCCTTGAAGTACTTTGACACCACCATCGACCGGAAGAGGTACACCAGC |
| | ACCAAAGAGGTGCTGGACGCCACCCTGATCCACCAGAGCATCACCGGCCTGTACGAG |
| | ACACGGATCGACCTGTCTCAGCTGGGAGGCGACAAGCGTCCTGCTGCTACTAAGAAA |
| | GCTGGTGAAGCTAAGAAAAAGAAAGCTAGCGGCAGCGGCGCCGGATCCCCAAAGAA |
| | GAAAAGGAAGGTTGAAGACCCCAAGAAAAAGAGGAAGGTGATACCCGGGTAAGCGG |
| | GACTCTGGGGTTCGAAATGACCGACCAAGCGACGCCCAACCTGCCATCACGAGATTT |
| | CGATTCCACCGCCGCCTTCTATGAAAGGTTGGGCTTCGGAATCGTTTTCCGGGACGCC |
| | GGCTGGATGATCCTCCAGCGCGGGGATCTCATGCTGGAGTTCTTCGCCCACCCTAGGG |
| | GGAGGCTAACTGAAACACGGAAGGAGACAATACCGGAAGGAACCCGCGCTATGACG |
| | GCAATAAAAAGACAGAATAAAACGCACGGTGTTGGGTCGTTTGTTCATAAACGCGGG |
| | GTTCGGTCCCAGGGCTGGCACTCTGTCGATACCCCACCGAGACGCCATTGGGGCCAAT |
| | ACGCCCGCGTTTCTTCCTTTTCCCCACCCCACCCCCCAAGTTCGGGTGAAGGCCCAGG |
| | GCTCGCAGCCAACGTCGGGGCGGCAGGCCCTGCCATAGCCTCAG |
| Construct 8- | TGTACAAAAAAGCAGGCTTTAAAGGAACCAATTCAGTCGACTGGATCCGGTACCAAG |
| U6p_30ntr_stgRNA | GTCGGGCAGGAAGAGGGCCTATTTCCCATGATTCCTTCATATTTGCATATACGATACA |
| SEQ ID NO: 15 | AGGCTGTTAGAGAGATAATTAGAATTAATTTGACTGTAAACACAAAGATATTAGTAC |
| | AAAATACGTGACGTAGAAAGTAATAATTTCTTGGGTAGTTTGCAGTTTTAAAATTATG |
| | TTTTAAAATGGACTATCATATGCTTACCGTAACTTGAAAGTATTTCGATTTCTTGGCTT |
| | TATATATCTTGTGGAAAGGACGAAACACCGCGGTCTGCGATAAGTCGGAGTACTGTCC |
| | TGGGTTAGAGCTAGAAATAGCAAGTTAACCTAAGGCTAGTCCGTTATCAACTTGAAA |
| | AAGTGGCACCGAGTCGGTGCTTTTT |
| Construct 9- | TGTACAAAAAAGCAGGCTTTAAAGGAACCAATTCAGTCGACTGGATCCGGTACCAAG |
| U6p_30nt_stgRNA | GTCGGGCAGGAAGAGGGCCTATTTCCCATGATTCCTTCATATTTGCATATACGATACA |
| SEQ ID NO: 16 | AGGCTGTTAGAGAGATAATTAGAATTAATTTGACTGTAAACACAAAGATATTAGTAC |
| | AAAATACGTGACGTAGAAAGTAATAATTTCTTGGGTAGTTTGCAGTTTTAAAATTATG |
| | TTTTAAAATGGACTATCATATGCTTACCGTAACTTGAAAGTATTTCGATTTCTTGGCTT |
| | TATATATCTTGTGGAAAGGACGAAACACCGCAAATACCTCACACACTCCCAATACATG |
| | AAGGGTTAGAGCTAGAAATAGCAAGTTAACCTAAGGCTAGTCCGTTATCAACTTGAA |
| | AAAGTGGCACCGAGTCGGTGCTTTTT |
| Construct 10- | TGTACAAAAAAGCAGGCTTTAAAGGAACCAATTCAGTCGACTGGATCCGGTACCAAG |
| U6p_40nt_stgRNA | GTCGGGCAGGAAGAGGGCCTATTTCCCATGATTCCTTCATATTTGCATATACGATACA |
| SEQ ID NO: 17 | AGGCTGTTAGAGAGATAATTAGAATTAATTTGACTGTAAACACAAAGATATTAGTAC |
| | AAAATACGTGACGTAGAAAGTAATAATTTCTTGGGTAGTTTGCAGTTTTAAAATTATG |
| | TTTTAAAATGGACTATCATATGCTTACCGTAACTTGAAAGTATTTCGATTTCTTGGCTT |
| | TATATATCTTGTGGAAAGGACGAAACACCGTCACCACATTATATCAATTACTTCTTAA |
| | ATCACACAATCAGGGTTAGAGCTAGAAATAGCAAGTTAACCTAAGGCTAGTCCGTTA |
| | TCAACTTGAAAAAGTGGCACCGAGTCGGTGCTTTTTT |
| Construct 11- | TGTACAAAAAAGCAGGCTTTAAAGGAACCAATTCAGTCGACTGGATCCGGTACCAAG |
| U6p_70nt_stgRNA | GTCGGGCAGGAAGAGGGCCTATTTCCCATGATTCCTTCATATTTGCATATACGATACA |
| SEQ ID NO: 18 | AGGCTGTTAGAGAGATAATTAGAATTAATTTGACTGTAAACACAAAGATATTAGTAC |
| | AAAATACGTGACGTAGAAAGTAATAATTTCTTGGGTAGTTTGCAGTTTTAAAATTATG |
| | TTTTAAAATGGACTATCATATGCTTACCGTAACTTGAAAGTATTTCGATTTCTTGGCTT |
| | TATATATCTTGTGGAAAGGACGGAACACCGCAAATACCTCACACACTCCCAATACATG |
| | AATCACCACATTATATCAATTACTTCTTAAATCACACAATCAGGGTTAGAGCTAGAAA |
| | TAGCAAGTTAACCTAAGGCTAGTCCGTTATCAACTTGAAAAAGTGGCACCGAGTCGGT |
| | GCTTTTTT |
| Construct 12- | GCGCCGGGTTTTGGCGCCTCCCGCGGGCGCCCCCCTCCTCACGGCGAGCGCTGCCACG |
| hUBCp_Cas9_3xNLS_p2a_puro | TCAGACGAAGGGCGCAGCGAGCGTCCTGATCCTTCCGCCCGGACGCTCAGGACAGCG |
| R | GCCCGCTGCTCATAAGACTCGGCCTTAGAACCCCAGTATCAGCAGAAGGACATTTTAG |
| SEQ ID NO: 19 | GACGGGACTTGGGTGACTCTAGGGCACTGGTTTTCTTTCCAGAGAGCGGAACAGGCG |
| | AGGAAAAGTAGTCCCTTCTCGGCGATTCTGCGGAGGGATCTCCGTGGGGCGGTGAAC |
| | GCCGATGATTATATAAGGACGCGCCGGGTGTGGCACAGCTAGTTCCGTCGCAGCCGG |
| | GATTTGGGTCGCGGTTCTTGTTTGTGGATCGCTGTGATCGTCACTTGGTGAGTAGCGG |
| | GCTGCTGGGCTGGCCGGGGCTTTCGTGGCCGCCGGGCCGCTCGGTGGGACGGAAGCG |
| | TGTGGAGAGACCGCCAAGGGCTGTAGTCTGGGTCCGCGAGCAAGGTTGCCCTGAACT |
| | GGGGGTTGGGGGGAGCGCAGCAAAATGGCGGCTGTTCCCGAGTCTTGAATGGAAGAC |
| | GCTTGTGAGGCGGGCTGTGAGGTCGTTGAAACAAGGTGGGGGGCATGGTGGGCGGCA |
| | AGAACCCAAGGTCTTGAGGCCTTCGCTAATGCGGGAAAGCTCTTATTCGGGTGAGATG |
| | GGCTGGGGCACCATCTGGGGACCCTGACGTGAAGTTTGTCACTGACTGGAGAACTCG |
| | GGTTTGTCGTCTGTTGCGGGGGCGGCAGTTATGGCGGTGCCGTTGGGCAGTGCACCCG |
| | TACCTTTGGGAGCGCGCGCCCTCGTCGTGTCGTGACGTCACCCGTTCTGTTGGCTTATA |
| | ATGCAGGGTGGGGCCACCTGCCGGTAGGTGTGCGGTAGGCTTTTCTCCGTCGCAGGAC |
| | GCAGGGTTCGGGCCTAGGGTAGGCTCTCCTGAATCGACAGGCGCCGGACCTCTGGTG |
| | AGGGGAGGGATAAGTGAGGCGTCAGTTTCTTTGGTCGGTTTTATGTACCTATCTTCTT |
| | AAGTAGCTGAAGCTCCGGTTTTGAACTATGCGCTCGGGGTTGGCGAGTGTGTTTTGTG |
| | AAGTTTTTTAGGCACCTTTTGAAATGTAATCATTTGGGTCAATATGTAATTTTCAGTGT |
| | TAGACTAGTAAATTGTCCGCTAAATTCTGGCCGTTTTTGGCTTTTTTGTTAGACGAAGC |
| | TTGGGCTGCAGGTCGACTCTAGAGGATCCCCGGGTACCGGTCGCCAACGCGTGCCACC |
| | ATGGACAAGAAGTACAGCATCGGCCTGGACATCGGCACCAACTCTGTGGGCTGGGCC |
| | GTGATCACCGACGAGTACAAGGTGCCCAGCAAGAAATTCAAGGTGCTGGGCAACACC |
| | GACCGGCACAGCATCAAGAAGAACCTGATCGGAGCCCTGCTGTTCGACAGCGGCGAA |
| | ACAGCCGAGGCCACCCGGCTGAAGAGAACCGCCAGAAGAAGATACACCAGACGGAA |
| | GAACCGGATCTGCTATCTGCAAGAGATCTTCAGCAACGAGATGGCCAAGGTGGACGA |
| | CAGCTTCTTCCACAGACTGGAAGAGTCCTTCCTGGTGGAAGAGGATAAGAAGCACGA |
| | GCGGCACCCCATCTTCGGCAACATCGTGGACGAGGTGGCCTACCACGAGAAGTACCC |
| | CACCATCTACCACCTGAGAAAGAAACTGGTGGACAGCACCGACAAGGCCGACCTGCG |
| | GCTGATCTATCTGGCCCTGGCCCACATGATCAAGTTCCGGGGCCACTTCCTGATCGAG |
| | GGCGACCTGAACCCCGACAACAGCGACGTGGACAAGCTGTTCATCCAGCTGGTGCAG |
| | ACCTACAACCAGCTGTTCGAGGAAAACCCCATCAACGCCAGCGGCGTGGACGCCAAG |
| | GCCATCCTGTCTGCCAGACTGAGCAACAGCAGACGGCTGGAAAATCTGATCGCCCAG |
| | CTGCCCGGCGAGAAGAAGAATGGCCTGTTCGGAAACCTGATTGCCCTGAGCCTGGGC |
| | CTGACCCCCAACTTCAAGAGCAACTTCGACCTGGCCGAGGATGCCAAACTGCAGCTG |
| | AGCAAGGACACCTACGACGACGACCTGGACAACCTGCTGGCCCAGATGGGCGACCAG |
| | TACGCCGACCTGTTTCTGGCCGCCAAGAACCTGTCCGACGCCATCCTGCTGAGCGACA |
| | TCCTGAGAGTGAACACCGAGATCACCAAGGCCCCCCTGAGCGCCTCTATGATCAAGA |
| | GATACGACGAGCACCACCAGGACCTGACCCTGCTGAAAGCTCTCGTGCGGCAGCAGC |
| | TGCCTGAGAAGTACAAAGAGATTTTCTTCGACCAGAGCAAGAACGGCTACGCCGGCr |
| | ACATTGACGGCGGAGCCAGCCAGGAAGAGTTCTACAAGTTCATCAAGCCCATCCTGG |
| | AAAAGATGGACGGCACCGAGGAACTGCTCGTGAAGCTGAACAGAGAGGACCTGCTGC |
| | GGAAGCAGCGGACCTTCGACAACGGCAGCATCCCCCACCAGATCCACCTGGGAGAGC |
| | TGCACGCCATTCTGCGGCGGCAGGAAGATTTTTACCCATTCCTGAAGGACAACCGGGA |
| | AAAGATCGAGAAGATCCTGACCTTCCGCATCCCCTACTACGTGGGCCCTCTGGCCAGG |
| | GGAAACAGCAGATTCGCCTGGATGACCAGAAAGAGCGAGGAAACCATCACCCCCTGG |
| | AACTTCGAGGAAGTGGTGGACAAGGGCGCTTCCGCCCAGAGCTTCATCGAGCGGATG |
| | ACCAACTTCGATAAGAACCTGCCCAACGAGAAGGTGCTGCCCAAGCACAGCCTGCTG |
| | TACGAGTACTTCACCGTGTATAACGAGCTGACCAAAGTGAAATACCTGACCGAGGGA |
| | ATGAGAAAGCCCGCCTTCCTGAGCGGCGAGCAGAAAAAGGCCATCGTGGACCTGCTG |
| | TTCAAGACCAACCGGAAAGTGACCGTGAAGCAGCTGAAAGAGGACTACTTCAAGAAA |
| | ATCGAGTGCTTCGACTCCGTGGAAATCTCCGGCGTGGAAGATCGGTTCAACGCCTCCC |
| | TGGGCACATACCACGATCTGCTGAAAATTATCAAGGACAAGGACTTCCTGCACAATG |
| | AGGAAAACGAGGACATTCTGGAAGATATCGTGCTGACCCTGACACTGTTTGAGGACA |
| | GAGAGATGATCGAGGAACGGCTGAAAACCTATGCCCACCTGTTCGACGACAAAGTGA |
| | TGAAGCAGCTGAAGCGGCGGAGATACACCGGCTGGGGCAGGCTGAGCCGGAAGCTG |
| | ATCAACGGCATCCGGGACAAGCAGTCCGGCAAGACAATCCTGGATTTCCTGAAGTCC |
| | GACGGCTTCGCCAACAGAAACTTCATGCAGCTGATCCACGACGACAGCCTGACCnTA |
| | AAGAGGACATCCAGAAAGCCCAGGTGTCCGGCCAGGGCGATAGCCTGCACGAGCACA |
| | TTGCCAATCTGGCCGGCAGCCCCGCCATTAAGAAGGGCATCCTGCAGACAGTGAAGG |
| | TGGTGGACGAGCTCGTGAAAGTGATGGGCCGGCACAAGCCCGAGAACATCGTGATCG |
| | AAATGGCCAGAGAGAACCAGACCACCCAGAAGGGACAGAAGAACAGCCGCGAGAGA |
| | ATGAAGCGGATCGAAGAGGGCATCAAAGAGCTGGGCAGCCAGATCCTGAAAGAACA |
| | CCCCGTGGAAAACACCCAGCTGCAGAACGAGAAGCTGTACCTGTACTACCTGCAGAA |
| | TGGGCGGGATATGTACGTGGACCAGGAACTGGACATCAACCGGCTGTCCGACTACGA |
| | TGTGGACCATATCGTGCCTCAGAGCTTTCTGAAGGACGACTCCATCGACAACAAGGTG |
| | CTGACCAGAAGCGACAAGAACCGGGGCAAGAGCGACAACGTGCCCTCCGAAGAGGT |
| | CGTGAAGAAGATGAAGAACTACTGGCGGCAGCTGCTGAACGCCAAGCTGATTACCCA |
| | GAGAAAGTTCGACAATCTGACCAAGGCCGAGAGAGGCGGCCTGAGCGAACTGGATA |
| | AGGCCGGCTTCATCAAGAGACAGCTGGTGGAAACCCGGCAGATCACAAAGCACGTGG |
| | CACAGATCCTGGACTCCCGGATGAACACTAAGTACGACGAGAATGACAAGCTGATCC |
| | GGGAAGTGAAAGTGATCACCCTGAAGTCCAAGCTGGTGTCCGATTTCCGGAAGGATT |
| | TCCAGTTTTACAAAGTGCGCGAGATCAACAACTACCACCACGCCCACGACGCCTACCT |
| | GAACGCCGTCGTGGGAACCGCCCTGATCAAAAAGTACCCTAAGCTGGAAAGCGAGTT |
| | CGTGTACGGCGACTACAAGGTGTACGACGTGCGGAAGATGATCGCCAAGAGCGAGCA |
| | GGAAATCGGCAAGGCTACCGCCAAGTACTTCTTCTACAGCAACATCATGAACTTTTTC |
| | AAGACCGAGATTACCCTGGCCAACGGCGAGATCCGGAAGCGGCCTCTGATCGAGACA |
| | AACGGCGAAACCGGGGAGATCGTGTGGGATAAGGGCCGGGATTTTGCCACCGTGCGG |
| | AAAGTGCTGAGCATGCCCCAAGTGAATATCGTGAAAAAGACCGAGGTGCAGACAGGC |
| | GGCTTGAGCAAAGAGTCTATGCTGCCCAAGAGGAACAGCGATAAGCTGATCGCCAGA |
| | AAGAAGGACTGGGACCCTAAGAAGTACGGCGGCTTCGACAGCCCCACCGTGGCCTAT |
| | TCTGTGCTGGTGGTGGCCAAAGTGGAAAAGGGCAAGTCCAAGAAACTGAAGAGTGTG |
| | AAAGAGCTGCTGGGGATCACCATCATGGAAAGAAGCAGCTTCGAGAAGAATCCCATC |
| | GACTTTCTGGAAGCCAAGGGCTACAAAGAAGTGAAAAAGGACCTGATCATCAAGCTG |
| | CCTAAGTACTCCCTGTTCGAGCTGGAAAACGGCCGGAAGAGAATGCTGGCCTCTGCC |
| | GGCGAACTGCAGAAGGGAAACGAACTGGCCCTGCCCTCCAAATATGTGAACTTCCTG |
| | TACCTGGCCAGCCACTATGAGAAGCTGAAGGGCTCCCCCGAGGATAATGAGCAGAAA |
| | CAGCTGTTTGTGGAACAGCACAAGCACTACCTGGACGAGATCATCGAGCAGATCAGC |
| | GAGTTCTCCAAGAGAGTGATCCTGGCCGACGCTAATCTGGACAAAGTGCTGTCCGCCT |
| | ACAACAAGCACCGGGATAAGCCCATCAGAGAGCAGGCCGAGAATATCATCCACCTGT |
| | TTACCCTGACCAATCTGGGAGCCCCTGCCGCCTTCAAGTACTTTGACACCACCATCGA |
| | CCGGAAGAGGTACACCAGCACCAAAGAGGTGCTGGACGCCACCCTGATCCACCAGAG |
| | CATCACCGGCCTGTACGAGACACGGATCGACCTGTCTCAGCTGGGAGGCGACAAGCG |
| | TCCTGCTGCTACTAAGAAAGCTGGTCAAGCTAAGAAAAAGAAAGCTAGCGGCAGCGG |
| | CGCCGGATCCCCAAAGAAGAAAAGGAAGGTTGAAGACCCCAAGAAAAAGAGGAAGG |
| | TGATAAGCGCTGGAAGCGGAGCTACTAACTTCAGCCTGCTGAAGCAGGCTGGAGACG |
| | TGGAGGAGAACCCTGGACCTACCGAGTACAAGCCCACGGTGCGCCTCGCCACCCGCG |
| | ACGACGTCCCCCGGGCCGTACGCACCCTCGCCGCCGCGTTCGCCGACTACCCCGCCAC |
| | GCGCCACACCGTCGACCCGGACCGCCACATCGAGCGGGTCACCGAGCTGCAAGAACT |
| | CTTCCTCACGCGCGTCGGGCTCGACATCGGCAAGGTGTGGGTCGCGGACGACGGCGC |
| | CGCGGTGGCGGTCTGGACCACGCCGGAGAGCGTCGAAGCGGGGGCGGTGTTCGCCGA |
| | GATCGGCCCGCGCATGGCCGAGTTGAGCGGTTCCCGGCTGGCCGCGCAGCAACAGAT |
| | GGAAGGCCTCCTGGCGCCGCACCGGCCCAAGGAGCCCGCGTGGTTCCTGGCCACCGT |
| | CGGCGTTTTCGCCCGACCACCAGGGCAAGGGTGTGGGCAGCGCCGTCGTGCTCCCCGG |
| | AGTGGAGGCGGCCGAGCGCGCCGGGGTGCCCGCCTTCCTGGAGACATCCGCGCCCCG |
| | CAACCTCCCCTTCTACGAGCGGCTCGGCTTCACCGTCACCGCCGACGTCGAGGTGCCC |
| | GAAGGACCGCGCACCTGGTGCATGACCCGCAAGCCCGGTGCCTGA |
| Construct 13- | TAGTTATTAATAGTAATCAATTACGGGGTCATTAGTTCATAGCCCATATATGGAGTC |
| CMVp_U6p_27nt1_GFP(+3)_ | CGCGTTACATAACTTACGGTAAATGGCCCGCCTGGCTGACCGCCCAACGACCCCCGCC |
| RFP(+2) | CATTGACGTCAATAATGACGTATGTTCCCATAGTAACGCCAATAGGGACTTTCCATTG |
| SEQ ID NO: 20 | ACGTCAATGGGTGGAGTTATTTACGGTTAAACTGCCCACTTGGCTAGTTACATCGTGAT |
| | CATATGCCAAGTACGCCCCCTATTGACGTCAATGACGGTAAATGGCCCGCCTGGCATT |
| | ATGCCCAGTACATGACCTTATGGGACTTTCCTACTTGGCAGTACATCTACGTATTAGTC |
| | ATCGCTATTACCATGGTGATGCGGTTTTGGCAGTACATCAATGGGCGTGGATAGCGGT |
| | TTGACTCACGGGGATTTCCAAGTCTCCACCCCATTGACGTCAATGGGAGTTTGTTTTG |
| | GCACCAAAATCAACGGGACTTTCCAAAATGTCGTAACAACTCCGCCCCATTGACGCA |
| | AATGGGCGGTAGGCGTGACGGTGGGTAGGTCTTATTATATAGCAGAGCTGGTTTAGGTA |
| | CCGTTCAGATCCTCTAGAGGATTCCCCGGGTTACCGGTCGCCACCTATGCCGAAAAGCC |
| | ACCTTGTACAAAAAAGCAGGCTTTAAAGGAACCAATTCAGTCGACTGGATCCGGTAC |
| | CAAGGTCGGGCTAGGTAAGAGGGCCTATTTTCCCTATGTATTTCCTTCTATTGCTATGTA |
| | TTACAAGGCTGTTCGAGAGATAATTTGAATTTATTTGACTGTAAACACAAAGATTATTTAG |
| | TACAAAATACGTGACGTCGAAAGTAATAATTTCTTGGGTAGTTTGCAGTTTTAAAATT |
| | ATGTTTTTTATATATGGACTTATCATATGCTTTACCGTTATACTTGATATAGGATTTTGG |
| | CTTTATATATCTTGTGGAAAGGACGAAACACCGATTCATCTCATCTATCAGAAACAAC |
| | AGGGTTGGTAGCTATAGTATAATTGCTAAGTCTAACCTTATAGATCTATACTTGCTATA |
| | AAGTGGCACCGAGTCGGTGCTTTTTTACCGGAAGCGGAGCTACTCACTTCAGCCTGCT |
| | GAAGCAGGCTGGAGACGTGGAGGAGAACCCTGGACCTG1GAGCAAGGGCGAGGAGC |
| | TGTTCACCGGGGTGGTGCCCATCCTGGTCGAGCTGGACGGCGACGTAAACGGCCACA |
| | AGTTCAGCGTGTCCGGCGAGGGCGAGGGCGATGCCACCTACGGCAAGCTGACCCTGA |
| | AGTTCATCTGCACCACCGGCAAGCTGCCCGTGCrCTGGCCCACCCTCGTGACCACCCT |
| | GACCTACGGCGTGCAGTGCTTCAGCCGCTACCCCGACCACATGAAGCAGCACGACTTC |
| | TTCAAGTCCGCCATGCCCGAAGGCTACGTCCAGGAGCGCACCATCTTCTTCAAGGACG |
| | ACGGCAACTACAAGACCCGCGCCGAGGTGAAGTTCGAGGGCGACACCCTGGTGAACC |
| | GCATCGAGCTGAAGGGCATCGACTTCAAGGAGGACGGCAACATCCTGGGGCACAAGC |
| | TGGTAGTTACTAACTTACTATACTAGCCACATACGTCTTATTATCTAGTATAGTATACG |
| | GCATCATAGGTGAACTTCAAGTATCCGCCACAACATCGTAGGACGGCAGCGTGCAGCTCG |
| | CCGACCACTTACCAGCAGAACACCCCCATCGGCGACGGCCCCGTGCTGCTGCCCGACA |
| | ACCTACTACCTTGTAGCTACCCTAGTCCGCCCTGAGCTATAAGTACCCCTGCGTATTT |
| | ACATGGTCCTGCTGGAGTTCGTGACCGCCGCCGGGATCACTCTCGGCATGGACGAGCT |
| | GTTACTAAGTTAAGGCCGGCCTAGCCACGGCTTCCCCCCTGTAGGTTGGCCGCGTACGTAT |
| | GGCTACCCTGCCCTATGTAGCTGCGCCCAGGTAGTAGCGGCTATGGTACTAGCCGCCGCT |
| | TGCGCCAGCGCTAGGATCAACGTGGGTGAGGGCAGAGGAAGTCTTCTAACTATGCGGT |
| | GACGTGGAGGAGAATCCGGGCCCTGTGAGCAAGGGCGAGGAGGATAACTCCGCCATC |
| | ATCAAGGAGTTCCTGCGCTTCAAGGTGCACATGGAGGGCTCCGTGAACGGCCACGAG |
| | TTCGAGATCGAGGGCGAGGGCGAGGGCCGCCCCTACGAGGGCACCCAGACCGCCAAG |
| | CTGAAGGTGACCAAGGGTGGCCCCCTGCCCTTCGCCTGGGACATCCTGTCCCCTCAGT |
| | TCATGTACGGCTCCAAGGCCTACGTGATAGCACCCCGCCGACATCCCCGACTACTTGAA |
| | GCTGTCCTTCCCCGAGGGCTTCAAGTGGGAGCGCGTGATGAACTTCGAGGACGGCGG |
| | CGTGGTGACCGTGACCCAGGACTCCTCTCTGCAGGACGGCGAGTTCATCTACAAGGTG |
| | AAGCTGCGCGGCACCAACTTCCCCTCCGACGGCCCCGTAATGCAGAAGAAGACCATG |
| | GGCTGGGAGGCCTCCTCCGAGCGGATGTACCCCGAGGACGGCGCCCTGAAGGGCGAG |
| | ATCAAGCAGAGGCTGAAGCTGAAGGACGGCGGCCACTACGACGCTGAGGTCAAGACC |
| | ACCTTTACTAAGGCCAAGAAGCCCGTTGCAGCTTGCCCGGCGCCTTACAACCTCAACCAAG |
| | TTGGACATCACCTCCCACAACGAGGACTACACCATCGTGGAACAGTACGAACGCGCC |
| | GAGGGCCGCCACTCCACCGGCGGCATGGACGAGCTGTACAAGTGA |
| Construct 14- | TAGTTATTAATAGTAATCAATTACGGGGTCATTAGTTCATAGCCCATATATGGAGTTC |
| CMVp_U6p_26nt1_GFP(+2)_ | CGCGTTACATAACTTACGGTAAATGGCCCGCCTGGCTGACCGCCCAACGACCCCCGCC |
| RFP(+1) | CATTGACGTCAATAATGACGTATGTTCCCATAGTAACGCCAATAGGGACTTTCCATTG |
| SEQ ID NO: 21 | ACGTCAATGGGTGGAGTATTTACGGTAAACTGCCCACTTGGCAGTACATCAAGTGTAT |
| | CATATGCCAAGTACGCCCCCTATTGACGTCAATGACGGTAAATGGCCCGCCTGGCATT |
| | ATGCCCAGTACATGACCTTATGGGACTTTCCTACTTGGCAGTACATCTACGTATTAGTC |
| | ATCGCTATTACCATGGTGATGCGGTTTTGGCAGTACATCAATGGGCGTGGATAGCGGT |
| | TTGACTCACGGGGATTTCCAAGTCTCCACCCCATTGACGTCAATGGGAGTTTGTTTTG |
| | GCACCAAAATCAACGGGACTTTCCAAAATGTCGTAACAACTCCGCCCCATTGACGCA |
| | AATGGGCGGTAGGCGTGTACGGTGGGAGGTCTATATAAGCAGAGCTGGTTTAGTGAA |
| | CCGTCAGATCCTCTAGAGGATCCCCGGGTACCGGTCGCCACCATGCCGAAAAGTGCC |
| | ACCTTGTACAAAAAAGCAGGCTTTAAAGGAACCAATTCAGTCGACTGGATCCGGTAC |
| | CAAGGTCGGGCAGGAAGAGGGCCTATTTCCCATGATTCCTTCATATTTGCATATACGA |
| | TACAAGGCTGTTCGAGAGATAATTTGAATTTATTTGACTGTAAACACAAAGATATTAG |
| | TACAAAATACGTGACGTCGAAAGTAATAATTTCTTGGGTAGTTTGCAGTTTTAAAATT |
| | ATGTTTTTAAATGGACTATCATATGCTTACCGTAACTTGAAAGTATTTCGATTTCTTGG |
| | CTTTATATATCTTGTGGAAAGGACGAAACACCGTTCATCTCATCTATCAGAAACAACA |
| | GGGTTGGAGCAAGAAATTGCAAGTCAACCTAAGGCTAGTCCGTTATCAACTTGCAAA |
| | AGTGGCACCGAGTCGGTGCTTTTTTACCGGAAGCGGAGCTACTCACTTCAGCCTGCTG |
| | AAGCAGGCTGGAGACGTGGAGGAGAACCCTGGACCTGTGAGCAAGGGCGAGGAGCT |
| | GTTCACCGGGGTGGTGCCCATCCTGGTGGAGCTGGACGGCGACGTAAACGGCCACAA |
| | GTTCAGCGTGTCCGGCGAGGGCGAGGGCGATGCCACCTACGGCAAGCTGACCCTGAA |
| | GTTCATCTGCACCACCGGCAAGCTGCCCGTGCCCTGGCCCACCCTCGTGACCACCCTG |
| | ACCTACGGCGTGCAGTGCTTCAGCCGCTACCCCGACCACATGAAGCAGCACGACTTCT |
| | TCAAGTCCGCCATGCCCGAAGGCTACGTCCAGGAGCGCACCATCTTCTTCAAGGACGA |
| | CGGCAACTACAAGACCCGCGCCGAGGTGAAGTTCGAGGGCGACACCCTGGTGAACCG |
| | CATCGAGCTGAAGGGCATCGACTTCAAGGAGGACGGCAACATCCTGGGGCACAAGCT |
| | GGAGTACAACTACAACAGCCACAACGTCTATATCATGGCCGACAAGCAGAAGAACGG |
| | CATCAAGGTGAACTTCAAGATCCGCCACAACATCGAGGACGGCAGCGTGCAGCTCGC |
| | CGACCACTACCAGCAGAACACCCCCATCGGCGACGGCCCCGTGCTGCTGCCCGACAA |
| | CCACTACCTGAGCACCCAGTCCGCCCTGAGCAAAGACCCCAACGAGAAGCGCGATCA |
| | CATGGTCCTGCTGGAGTTCGTGACCGCCGCCGGGATCACTCTCGGCATGGACGAGCTG |
| | TACAAGTAAGGCCGGCCAGCCACGGCTTCCCCCCTGAGGTGGCCGCTCAGGACGATG |
| | GCACCCTGCCCATGAGCTGCGCCCAGGAGAGCGGCATGGACAGGCACCCCGCCGCTT |
| | GCGCCAGCGCTAGGATCAACGTGGGTGAGGGCAGAGGAAGTCTTCTAACATGCGGTG |
| | ACGTGGAGGAGAATCCGGGCCCTGTGAGCAAGGGCGAGGAGGATAACTCCGCCATCA |
| | TCAAGGAGTTCCTGCGCTTCAAGGTGCACATGGAGGGCTCCGTGAACGGCCACGAGT |
| | TCGAGATCGAGGGCGAGGGCGAGGGCCGCCCCTACGAGGGCACCCAGACCGCCAAG |
| | CTGAAGGTGACCAAGGGTGGCCCCCTGCCCTTCGCCTGGGACATCCTGTCCCCTCAGT |
| | TCATGTACGGCTCCAAGGCCTACGTGAAGCACCCCGCCGACATCCCCGACTACTTGAA |
| | GCTGTCCTTCCCCGAGGGCTTCAAGTGGGAGCGCGTGATGAACTTCGAGGACGGCGG |
| | CGTGGTGACCGTGACCCAGGACTCCTCTCTGCAGGACGGCGAGTTCATCTACAAGGTG |
| | AAGCTGCGCGGCACCAACTTCCCCTCCGACGGCCCCGTAATGCAGAAGAAGACCATG |
| | GGCTGGGAGGCCTCCTCCGAGCGGATGTACCCCGAGGACGGCGCCCTGAAGGGCGAG |
| | ATCAAGCAGAGGCTGAAGCTGAAGGACGGCGGCCACTACGACGCTGAGGTCAAGACC |
| | ACCTACAAGGCCAAGAAGCCCGTGCAGCTGCCCGGCGCCTACAACGTCAACATCAAG |
| | TTGGACATCACCTCCCACAACGAGGACTACACCATCGTGGAACAGTACGAACGCGCC |
| | GAGGGCCGCCACTCCACCGGCGGCATGGACGAGCTGTACAAGTGA |
| Construct 15- | TAGTTATTAATAGTAATCAATTACGGGGTCATTAGTTCATAGCCCATATATGGAGTTC |
| CMVp_U6p_25nt1_GFP(+1)_ | CGCGTTACATAACTTACGGTAAATGGCCCGCCTGGCTGACCGCCCAACGACCCCCGCC |
| RFP(+3) | CATTGACTTCAATAATGACGTATGTTCCCATAGTAACGCCAATAGGGACTTTCCATTG |
| SEQ ID NO: 22 | ACGTCAATGGGTGGAGTATTTACGGTAAACTGCCCACTTGGCAGTACATCAAGTGTAT |
| | CATATGCCAAGTACGCCCCCTATTGACGTCAATGACGGTAAATGGCCCGCCTGGCATT |
| | ATGCCCAGTACATGACCTTATGGGACTTTCCTACTTGGCAGTACATCTACGTATTAGTC |
| | ATCGCTATTACCATGGTGATGCGGTTTTGGCAGTACATCAATGGGCGTGGATAGCGGT |
| | TTGACTCACGCCCATTTCCAAGTCTCCACCCCATTGACGTCAATGGGAGTTTGTTTTG |
| | GCACCAAAATTAACGGGACTTTTAAAATGTCGTAACAACTCCGCCCCATTGACGCA |
| | AATGGGCGGTAGGCGTGTACCGTGGGAGGTCTATATAAGCAGAGCTGCTTTAGTGAA |
| | CCGTCAGATCCTCTAGAGGATCCCCGGGTACCGGTCGCCACCATGCCGAAAAGTGCC |
| | ACCTTGTACAAAAAAGCAGGCTTTAAAGGAACCAATTCAGTGGACTGGATTTGGTAC |
| | CAAGGTCGGGCAGGAAGAGGGCCTATTTCCCATGATTCCTTCATATTTGCATATACGA |
| | TACAAGGCTGTTCGAGAGATAATTTGAATTTATTTGACTGTAAACACAAAGATATTAG |
| | TACAAAATACGTGACGTCGAAAGTAATAATTTCTTGGGTAGTTTGCAGnTTAAAATT |
| | ATGTTTTTAAATGGACTATCATATGCTTACCGTAACTTGAAAGTATTTCGATTTCTTGG |
| | CTTTATATATCTTGTGGAAAGGACGAAACACCGTCATCTCATCTATCAGAAACAACAG |
| | GGTTGGAGCAAGAAATTGCAACTCAACCTAAGGCTAGTCCCTTATCAACTTGCAAAA |
| | GTGGCACCGAGTCGGTGCTTTTTTACCGGAAGCGGAGCTACTCACTTCAGCCTGCTGA |
| | AGCAGGCTGGAGACGTGGAGGAGAACCCTGGACCTGTGAGCAAGGGCGAGGAGCTG |
| | TTCACCGGGGTGGTGCCCATCCTGGTCGAGCTGGACGGCGACGTAAACGGCCACAAG |
| | TTCAGCGTGTCCGGCGAGGGCGAGGGCGATGCCACCTACGGCAAGCTGACCCTGAAG |
| | TTCATCTGCACCACCGGCAAGCTGCCCGTGCCCTGGCCCACCCTCGTGACCACCCTGA |
| | CCTACGGCGTGCAGTGCTTCAGCCGCTACCCCGACCACATGAAGCAGCACGACTTCTT |
| | CAAGTCCGCCATGCCCGAAGGCTACGTCCAGGAGCGCACCATCTTCTTCAAGGACGA |
| | CGGCAACTACAACACCCGCGCCGAGGTGAAGTTCGAGGGCGACACCCTGGTGAACCG |
| | CATCGAGCTGAAGGGCATCGACTTCAAGGAGGACGGCAACATCCTGGGGCACAAGCT |
| | GGAGTACAACTACAACAGCCACAACGTCTATATCATGGCOGACAAGCAGAAGAACGG |
| | CATCAAGGTGAACTTCAAGATCCGCCACAACATCGAGGACGiGCAGCGTGCAGTCTCGC |
| | CGACCACTACCAGCAGAACACCCCCATCGGCGACGGCCCCGTGCTGCTGCCCGACAA |
| | CCACTACCTGAGCACCCAGTCCGCCCTGAGCAAAGACCCCAACGAGAAGCGCGATCA |
| | CATGGTCCTGCTGGAGTTCGTGACCGCCGCCGGGATCACTCTCGGCATGGACCAGCTG |
| | TACAAGTAAGGCCGGCCAGCCACGGCTTCCCCCCTGAGGTGGCCGCTCAGGACGATC |
| | GCACCCTGCCCATGAGCTGCGCCCAGGAGAGCGGCATGGACAGGCACCCCGCCGCTT |
| | GCGCCAGCGCTAGGATCAACGTGGGTGAGGGCAGAGGAAGTCTTCTAACATGCGGTG |
| | ACGTGGAGGAGAATCCGGGCCCTGTGAGCAAGGGCGAGGAGGAGAACGCCGCCATCA |
| | TCAAGGAGTTCCTGCGCTTCAAGGTGCACATGGAGGGCTCCGTGAACGGCCACGAGT |
| | TCGAGATCGAGGGCGAGGGCGAGGGCCGCCCCTACCAGGGCACCCAGACCGCCAAG |
| | CTGAAGGTGACCAAGGGTGGCCCCCTGCCCTTCGCCTGGGACATCCTGTCCCCTCAGT |
| | TCATGTACGGCTCCAAGGCCTACGTGAAGCACCCCGCCGACATCCCCGACTACTTGAA |
| | GCTGTCCTTCCCCGAGGGCTTCAAGTGGGAGCGCGTGATGAACTTCGAGGACGGCGG |
| | CGTGGTGACCGTGACCCAGGACTCCTCTCTGCAGGACGGCGAGTTCATCTACAAGGTG |
| | AAGCTGCGCGGCACCAACTTCCCCTCCGACGGCCCCGTAATGCAGAAGAAGACCATG |
| | GGCTGGGAGGCCTCCTCCGAGCGGATGTACCCCGAGGACGGCGCCCTGAAGGGCGAG |
| | ATCAAGCAGAGGCTGAAGCTGAAGGACGGCGGCCACTACGACGCTGAGGTCAAGACC |
| | ACCTACAAGGCCAAGAAGCCCGTGCAGCTGCCCGGCGCCTACAACGTCAACATCAAC |
| | TTGCACATCACCTCCCACAACCAGCACTACACCATCCTGGAACAGTACCAACGCGCC |
| | GAGGGCCGCCACTCCACCGGCGGCATGGACGAGCTGTACAAGTGA |
| Construct 16- | TGTACAAAAAAGCAGGCTTTAAAGGAACCAATTCAGTCGCTGGATCCGGTACCAAG |
| U6p_27nt1_CMVp_target_GFP | GTCGGGCAGGAAGAGGGCCTATTTCCCATGATTCCTTCATATTTGCATATACGATACA |
| (+3)_RFP(+2) | AGGCTGTTAGAGAGATAATTAGAATTAATTTGACTGTAAACACAAAGATATTAGTAC |
| SEQ ID NO: 23 | AAAATACGTGACGTAGAAAGTAATAATTTCTTGGGTAGTTTGCAGTTTTAAAATTATG |
| | TTTTAAAATGGACTATCATATGCTTACCGTAACTTGAAAGTATTTCGATTTCTTGGCTT |
| | TATATATCTTGTGGAAAGGACGAAACACCGATTCATCTCATCTATCAGAAACAACAGT |
| | TTTAGAGCTAGAAATAGCAAGTTAAAATAAGGCTAGTCCGTTATCAACTTGAAAAAG |
| | TGGCACCGAGTCGGTGCTTTTTTTCTAGACCCAGCTTTCTTGTACAAAGTTGGCATTAG |
| | ACGTCGAGGCTAGCCCAGACTTAATTAATAGTTATTAATAGTAATCAATTACGGGGTC |
| | ATTAGTTCATAGCCCATATATGGAGTTCCGCGTTACATAACTTACGGTAAATGGCCCG |
| | CCTGGCTGACCGCCCAACGACCCCCGCCCATTGACGTCAATAATGACGTATGTTCCCA |
| | TAGTAACGCCAATAGGGACTTTCCATTGACGTCAATGGGTGGAGTATTTACGGTAAAC |
| | TCCCCACTTGGCAGTACATCAAGTGTATCATATGCCAAGTACGCCCCCTATTGACGTC |
| | AATGACGGTAAATGGCCCGCCTGGCATTATGCCCAGTACATGACCTTATGGGACTTTC |
| | CTACTTGGCAGTACATCTACGTATTAGTCATCGCTATTACCATGGTGATGCGGTTTTGG |
| | CAGTACATCAATGGGCGTGGATAGCGGTTTGACTCACGGGGATTTCCAAGTCTCCACC |
| | CCATTGACGTCAATGGGAGTTTGTTTTGGCACCAAAATCAACGGGACTTTCCAAAATG |
| | TCGTAACAACTCCGCCCCATTGACGCAAATGGGCGGTAGGCGTGTACGGTGGGAGGT |
| | CTATATAAGCAGAGCTGGTTTAGTGAACCGTCAGATCCTCTAGAGGATCCCCGGGTAC |
| | CGGTCGCCACCATGCCGAAAAGTGCCACCGATTTATCTCATCTATCAGAAACAACAG |
| | GGCCGGAAGCGGAGCTACTCACTTCAGCCTGCTGAAGCAGGCTGGAGACGTGGAGGA |
| | GAACCCTGGACCTGTGAGCAAGGGCGAGGAGCTGTTCACCGGGGTGGTGCCCATCCT |
| | GGTCGAGCTGGACGGCGACGTAAACGGCCACAAGTTCAGCGTGTCCTGGCGAGGGCGA |
| | GGGCGATGCCACCTACGGCAAGCTGACCCTGAAGTTCATCTGCACCACCGGCAAGCT |
| | GCCCGTGCCCTGGCCCACCCTCGTGACCACCCTGACCTACGGCGTGCAGTGCTTCAGC |
| | CGCTACCCCGACCACATGAAGCAGCACGACTTCTTCAAGTGCGCCATGCCCGAAGGCT |
| | ACGTCCAGGAGCGCACCATCTTCTTCAAGGACGACGGCAACTACAAGACCCGCGCCG |
| | AGGTGAAGTTTGAGGGCGACACCCTGGTGAACCGCATCGAGCTGAAGGGCATCGACT |
| | TCAAGGAGGACGGCAACATCCTGGGGCACAAGCTGGAGTACAACTACAACAGCCACA |
| | ACGTCTATATCATGGCCGACAAGCAGAAGAACGGCATCAAGGTGAACTTCAAGATCC |
| | GCCACAACATCGAGGACGGCAGCGTGCAGCTCGTCGACCACTACCAGCAGAACACCC |
| | CCATCGGCGACGGCCCCGTGCTGCTGCCCGACAACCACTACCTGAGCACCCAGTCCGC |
| | CCTGAGCAAAGACCCCAACGAGAAGCGCGATCACATGGTCCTGCTGGAGTTCGTGAC |
| | CGCCGCCGGGATCACTCTCGGCATGGACGAGCTGTACAAGTAAGGCCGGCCAGCCAC |
| | GGCTTCCCCCCTGAGGTGGCCGCTCAGGACGATGGCACCCTGCCCATGAGCTGCGCCC |
| | AGGAGAGCGGCATGGACAGGCACCCCGCCGCTTGCGCCAGCGCTAGGATCAACGTGG |
| | GTGAGGGCAGAGGAAGTCTTCTAACATGCGGTGACGTGGAGGAGAATCCGGGCCCTG |
| | TGAGCAAGGGCGAGGAGGATAACTCCGCCATCATCAAGGAGTTCCTGCGCTTCAAGG |
| | TGCACATGGAGGGCTCCGTGAACGGCCACGAGTTCGAGATCGAGGGCGAGGGCGAGG |
| | GCCGCCCCTACGAGGGCACCCAGACCGCCAAGCTGAAGGTGACCAAGGGTGGCCCCC |
| | TGCCCTTCGCCTGGGACATCCTGTCCCCTCAGTTCATGTACGGCTCCAAGGCCTACGT |
| | GAAGCACCCCGCCGACATCCCCGACTACTTGAAGCTGTCCTTCCCCGAGGGCTTCAAG |
| | TGGGAGCGCGTGATGAACTTCGAGGACGGCGGCGTGGTGACCGTGACCCAGGACTCC |
| | TCTCTGCAGGACGGCGAGTTCATCTACAAGGTGAAGCTGCGCGGCACCAACTTCCCCT |
| | CCGACGGCCCCGTAATGCAGAAGAAGACCATGGGCTGGGAGGCCTCCTCCGAGCGGA |
| | TGTACCCCGAGGACGGCGCCCTGAAGGGCGAGATCAAGCAGAGGCTGAAGCTGAAG |
| | GACGGCGGCCACTACGACGCTGAGGTCAAGACCACCTACAAGGCCAAGAAGCCCGTG |
| | CAGCTGCCCGGCGCCTACAACGTCAACATCAAGTTGGACATCACCTCCCACAACGAG |
| | GACTACACCATCGTGGAACAGTACGAACGCGCCGAGGGCCGCCACTCCACCGGCGGC |
| | ATGGACGAGCTGTACAAGTGA |
| Construct 17- | TGTACAAAAAAGCAGGCTTTAAAGGAACCAATTCAGTCGACTGGATCCGGTACCAAG |
| U6p_26nt1_CMVp_target_GFP | GTCGGGCAGGAAGAGGGCCTATTTCCCATGATTCCTTCATATTTGCATATACGATACA |
| (+2)_RFP(+1) | AGGCTGTTAGAGAGATAATTAGAATTAATTTGACTGTAAACACAAAGATATTAGTAC |
| SEQ ID NO: 24 | AAAATACGTGACGTAGAAAGTAATAATTTCTTGGGTAGTTTGCAGTTTTAAAATTATG |
| | TTTTAAAATGGACTATCATATGCTTACCGTAACTTGAAAGTATTTCGATTTCTTGGCTT |
| | TATATATCTTGTGGAAAGGACGAAACACCGATTCATCTCATCTATCAGAAACAACAGT |
| | TTTAGAGCTAGAAArAGCAAGTTAAAATAAGGCTAGTCCGTTATCAACTTGAAAAAG |
| | ACGTCGAGGCTAGCCCAGACTTAATTAATAGTTATTAATAGTAATCAATTACGGGGTC |
| | ATTAGTTCATAGCCCATATATGGAGTTCCGCGTTACATAACTTACGGTAAATGGCCCG |
| | CCTGGCTGACCGCCCAACGACCCCCGCCCATTGACGTCAATAATGACGTATGTTCCCA |
| | TAGTAACGCCAATAGGGACTTTCCATTGACGTCAATGGGTGGAGTATTTACGGTAAAC |
| | TGCCCACTTGGCAGTACATCAAGTGTATCATATGCCAAGTACGCCCCCTATTGACGTC |
| | AATGACGGTAAATGGCCCGCCTGGCATTATGCCCAGTACATGACCTTATGGGACTTTC |
| | CTACTTGGCAGTACATCTACGTATTAGTCATCGCTATTACCATGGTGATGCGGTTTTGG |
| | CAGTACATCAATGGGCGTGGATAGCGGTTTGACTCACGGGGATTTCCAAGTCTCCACC |
| | CCATTGACGTCAATGGGAGTTTGTTTTGGCACCAAAATCAACGGGACTTTCCAAAATG |
| | TCGTAACAACTCCGCCCCATTGACGCAAATGGGCGGTAGGCGTGTACGGTGGGAGGT |
| | CTATATAAGCAGAGCTGGTTTAGTGAACCGTCAGATCCTCTAGAGGATCCCCGGGTAC |
| | CGGTCGCCACCATGCCGAAAAGTGCCACCGTTCATCTCATCTATCAGAAACAACAGG |
| | GCCGGAAGCGGAGCTACTCACTTCAGCCTGCTGAAGCAGGCTGGAGACGTGGAGGAG |
| | AACCCTGGACCTGTGAGCAAGGGCGAGGAGCTGTTCACCGGGGTGGTGCCCATCCTG |
| | GTCGAGCTGGACGGCGACGTAAACGGCCACAAGTTCAGCGTGTCCGGCGAGGGCGAG |
| | GGCGATGCCACCTACGGCAAGCTGACCCTGAAGTTCATCTGCACCACCGGCAAGCTG |
| | CCCGTTTTCCTGGCCCACCCTCGTGACCACCCTGACCTACGGCGTGCAGTGCTTCAGCC |
| | GCTACCCCGACCACATGAAGCAGCACGACTTCTTCAAGTCCGCCATGCCCGAAGGCTA |
| Construct 17- | TGTACAAAAAAGCAGGCTTTAAAGGAACCAATTCAGTCGACTGGATCCGGTACCAAG |
| U6p_26nt1_CMVp_target GFP | GTCGGGCAGGAAGAGGGCCTATTTCCCATGATTCCTTCATATTTGCATATACGATACA |
| (+2)_RFP(+1) | AGGCTGTTAGAGAGATAATTAGAATTAATTTGACTGTAAACACAAAGATATTAGTAC |
| SEQ ID NO: 24 | AAAATACGTGACGTAGAAAGTAATAATTTCTTGGGTAGTTTGCAGTTTTAAAATTATG |
| | TTTTAAAATGGACTATCATATGCTTACCGTAACTTGAAAGTATTTCGATTTCTTGGCTT |
| | TATATATCTTGTGGAAAGGACGAAACACCGATTCATCTCATCTATCAGAAACAACAGT |
| | TTTAGAGCTAGAAATAGCAAGTTAAAATAAGGCTAGTCCGTTATCAACTTGAAAAAG |
| | TGGCACCGAGTCGGTGCTTTTTTTCTAGACCCAGCTTTCTTGTACAAAGTTGGCATTAG |
| | ACGTCGAGGCTAGCCCAGACTTAATTAATAGTTATTAATAGTAATCAATTACGGGGTC |
| | ATTAGTTCATAGCCCATATATGGAGTTCCGCGTTACATAACTTACGGTAAATGGCCCG |
| | CCTGGCTGACCGCCCAACGACCCCCGCCCATTGACGTCAATAATGACGTATGTTCCCA |
| | TAGTAACGCCAATAGGGACTTTCCATTGACGTCAATGGGTGGAGTATTTACGGTAAAC |
| | TGCCCACTTGGCAGTACATCAAGTGTATCATATGCCAAGTACGCCCCCTATTGACGTC |
| | AATGACGGTAAATGGCCCGCCTGGCATTATGCCCAGTACATGACCTTATGGGACTTTC |
| | CTACTTGGCAGTACATCTACGTATTAGTCATCGCTATTACCATGGTGATGCGGTTTTGG |
| | CAGTACATCAATGGGCGTGGATAGCGGTTTGACTCACGGGGATTTCCAAGTCTCCACC |
| | CCATTGACGTCAATGGGAGTTTGTTTTGGCACCAAAATCAACGGGACTTTCCAAAATG |
| | TCGTAACAACTCCGCCCCATTGACGCAAATGGGCGGTAGGCGTGTACGGTGGGAGGT |
| | CTATATAAGCAGAGCTGGTTTAGTGAACCGTCAGATCCTCTAGAGGATCCCCGGGTAC |
| | CGGTCGCCACCATGCCGAAAAGTGCCACCGTTCATCTCATCTATCAGAAACAACAGG |
| | GCCGGAAGCGGAGCTACTCACTTCAGCCTGCTGAAGCAGGCTGGAGACGTGGAGGAG |
| | AACCCTGGACCTGTGAGCAAGGGCGAGGAGCTGTTCACCGGGGTGGTGCCCATCCTG |
| | GTCGAGCTGGACGGCGACGTAAACGGCCACAAGTTCAGCGTGTCCGGCGAGGGCGAG |
| | GGCGATGCCACCTACGGCAAGCTGACCCTGAAGTTCATCTGCACCACCGGCAAGCTG |
| | CCCGTGCCCTGGCCCACCCTCGTGACCACCCTGACCTACGGCGTGCAGTGCTTCAGCC |
| | GCTACCCCGACCACATGAAGCAGCACGACTTCTTCAAGTCCGCCATGCCCGAAGGCTA |
| | CGTCCAGGAGCGCACCATCTTCTTCAAGGACGACGGCAACTACAAGACCCGCGCCGA |
| | GGTGAAGTTCGAGGGCGACACCCTGGTGAACCGCATCGAGCTGAAGGGCATCGACTT |
| | CAAGGAGGACGGCAACATCCTGGGGCACAAGCTGGAGTACAACTACAACAGCCACA |
| | ACGTCTATATCATGGCCGACAAGCAGAAGAACGGCATCAAGGTGAACTTCAAGATCC |
| | GCCACAACATCGAGGACGGCAGCGTGCAGCTCGCCGACCACTACCAGCAGAACACCC |
| | CCATCGGCGACGGCCCCGTGCTGCTGCCCGACAACCACTACCTGAGCACCCAGTCCGC |
| | CCTGAGCAAAGACCCCAACGAGAAGCGCGATCACATGGTCCTGCTGGAGTTCGTGAC |
| | CGCCGCCGGGATCACTCTCGGCATGGACGAGCTGTACAAGTAAGGCCGGCCAGCCAC |
| | GGCTTCCCCCCTGAGGTGGCCGCTCAGGACGATGGCACCCTGCCCATGAGCTGCGCCC |
| | AGGAGAGCGGCATGGACAGGCACCCCGCCGCTTGCGCCAGCGCTAGGATCAACGTGG |
| | GTGAGGGCAGAGGAAGTCTTCTAACATGCGGTGACGTGGAGGAGAATCCGGGCCCTG |
| | TGAGCAAGGGCGAGGAGGATAACTCCGCCATCATCAAGGAGTTCCTGCGCTTCAAGG |
| | TGCACATGGAGGGCTCCGTGAACGGCCACGAGTTCGAGATCGAGGGCGAGGGCGAGG |
| | GCCGCCCCTACGAGGGCACCCAGACCGCCAAGCTGAAGGTGACCAAGGGTGGCCCCC |
| | TGCCCTTCGCCTGGGACATCCTGTCCCCTCAGTTCATGTACGGCTCCAAGGCCTACGT |
| | GAAGCACCCCGCCGACATCCCCGACTACTTGAAGCTGTCCTTCCCCGAGGGCTTCAAG |
| | TGGGAGCGCGTGATGAACTTCGAGGACGGCGGCGTGGTGACCGTGACCCAGGACTCC |
| | TCTCTGCAGGACGGCGAGTTCATCTACAAGGTGAAGCTGCGCGGCACCAACTTCCCCT |
| | CCGACGGCCCCGTAATGCAGAAGAAGACCATGGGCTGGGAGGCCTCCTCCGAGCGGA |
| | TGTACCCCGAGGACGGCGCCCTGAAGGGCGAGATCAAGCAGAGGCTGAAGCTGAAG |
| | GACGGCGGCCACTACGACGCTGAGGTCAAGACCACCTACAAGGCCAAGAAGCCCGTG |
| | CAGCTGCCCGGCGCCTACAACGTCAACATCAAGTTGGACATCACCTCCCACAACGAG |
| | GACTACACCATCGTGGAACAGTACGAACGCGCCGAGGGCCGCCACTCCACCGGCGGC |
| | ATGGACGAGCTGTACAAGTGA |
| Construct 18- | TGTACAAAAAAGCAGGCTTTAAAGGAACCAATTCAGTCGACTGGATCCGGTACCAAG |
| U6P_25nt1_CMVp_target_GFP | GTCGGGCAGGAAGAGGGCCTATTTCCCATGATTCCTTCATATTTGCATATACGATACA |
| (+1)_RFP(+3) | AGGCTGTTAGAGAGATAATTAGAATTAATTTGACTGTAAACACAAAGATATTAGTAC |
| SEQ ID NO: 25 | AAAATACGTGACGTAGAAAGTAATAATTTCTTGGGTAGTTTGCAGTTTTAAAATTATG |
| | TTTTAAAATGGACTATCATATGCTTACCGTAACTTGAAAGTATTTCGATTTCTTGGCTT |
| | TATATATCTTGTGGAAAGGACGAAACACCGATTCATCTCATCTATCAGAAACAACAGT |
| | TTTAGAGCTAGAAATAGCAAGTTAAAATAAGGCTAGTCCGTTATCAACTTGAAAAAG |
| | TGGCACCGAGTCGGTGCTTTTTTTCTAGACCCAGCTTTCTTGTACAAAGTTGGCATTAG |
| | ACGTCGAGGCTAGCCCAGACTTAATTAATAGTTATTAATAGTAATCAATTACGGGGTC |
| | ATTAGTTCATAGCCCATATATGGAGTTCCGCGTTACATAACTTACGGTAAATGGCCCG |
| | CCTGGCTGACCGCCCAACGACCCCCGCCCATTGACGTCAATAATGACGTATGTTCCCA |
| | TAGTAACGCCAATAGGGACTTTCCATTGACGTCAATGGGTGGAGTATTTACGGTAAAC |
| | TGCCCACTTGGCAGTACATCAAGTGTATCATATGCCAAGTACGCCCCCTATTGACGTC |
| | AATGACGGTAAATGGCCCGCCTGGCATTATGCCCAGTACATGACCTTATGGGACTTTC |
| | CTACTTGGCAGTACATCTACGTATTAGTCATCGCTATTACCATGGTGATGCGGTTTTGG |
| | CAGTACATCAATGGGCGTGGATAGCGGTTTGACTCACGGGGATTTCCAAGTCTCCACC |
| | CCATTGACGTCAATGGGAGTTTGTTTTGGCACCAAAATCAACGGGACTTTCCAAAATG |
| | TCGTAACAACTCCGCCCCATTGACGCAAATGGGCGGTAGGCGTGTACGGTGGGAGGT |
| | CTATATAAGCAGAGCTGGTTTAGTGAACCGTCAGATCCTCTAGAGGATCCCCGGGTAC |
| | CGGTCGCCACCATGCCGAAAAGTGCCACCGTCATCTCATCTATCAGAAACAACAGGG |
| | CCGGAAGCGGAGCTACTCACTTCAGCCTGCTGAAGCAGGCTGGAGACGTGGAGGAGA |
| | ACCCTGGACCTGTGAGCAAGGGCGAGGAGCTGTTCACCGGGGTGGTGCCCATCCTGG |
| | TCGAGCTGGACGGCGACGTAAACGGCCACAAGTTCAGCGTGTCCGGCGAGGGCGAGG |
| | GCGATGCCACCTACGGCAAGCTGACCCTGAAGTTCATCTGCACCACCGGCAAGCTGCC |
| | CGTGCCCTGGCCCACCCTCGTGACCACCCTGACCTACGGCGTGCAGTGCTTCAGCCGC |
| | TACCCCGACCACATGAAGCAGCACGACTTCTTCAAGTCCGCCATGCCCGAAGGCTACG |
| | TCCAGGAGCGCACCATCTTCTTCAAGGACGACGGCAACTACAAGACCCGCGCCGAGG |
| | TGAAGTTCGAGGGCGACACCCTGGTGAACCGCATCGAGCTGAAGGGCATCGACTTCA |
| | AGGAGGACGGCAACATCCTGGGGCACAAGCTGGAGTACAACTACAACAGCCACAAC |
| | GTCTATATCATGGCCGACAAGCAGAAGAACGGCATCAAGGTGAACTTCAAGATCCGC |
| | ACAACATCGAGGACGGCAGCGTGCAGCTCGCCGACCACTACCAGCAGAACACCCCC |
| | ATCGGCGACGGCCCCGTGCTGCTGCCCGACAACCACTACCTGAGCACCCAGTCCGCCC |
| | TGAGCAAAGACCCCAACGAGAAGCGCGATCACATGGTCCTGCTGGAGTTCGTGACCG |
| | CCGCCGGGATCACTCTCGGCATGGACGAGCTGTACAAGTAAGGCCGGCCAGCCACGG |
| | CTTCCCCCCTGAGGTGGCCGCTCAGGACGATGGCACCCTGCCCATGAGCTGCGCCCAG |
| | GAGAGCGGCATGGACAGGCACCCCGCCGCTTGCGCCAGCGCTAGGATCAACGTGGGT |
| | GAGGGCAGAGGAAGTCTTCTAACATGCGGTGACGTGGAGGAGAATCCGGGCCCTGTG |
| | AGCAAGGGCGAGGAGGATAACTCCGCCATCATCAAGGAGTTCCTGCGCTTCAAGGTG |
| | CACATGGAGGGCTCCGTGAACGGCCACGAGTTCGAGATCGAGGGCGAGGGCGAGGG |
| | CCGCCCCTACGAGGGCACCCAGACCGCCAAGCTGAAGGTGACCAAGGGTGGCCCCCT |
| | GCCCTTCGCCTGGGACATCCTGTCCCCTCAGTTCATGTACGGCTCCAAGGCCTACGTG |
| | AAGCACCCCGCCGACATCCCCGACTACTTGAAGCTGTCCTTCCCCGAGGGCTTCAAGT |
| | GGGAGCGCGTGATGAACTTCGAGGACGGCGGCGTGGTGACCGTGACCCAGGACTCCT |
| | CTCTGCAGGACGGCGAGTTCATCTACAAGGTGAAGCTGCGCGGCACCAACTTCCCCTC |
| | CGACGGCCCCGTAATGCAGAAGAAGACCATGGGCTGGGAGGCCTCCTCCGAGCGGAT |
| | GTACCCCGAGGACGGCGCCCTGAAGGGCGAGATCAAGCAGAGGCTGAAGCTGAAGG |
| | ACGGCGGCCACTACGACGCTGAGGTCAAGACCACCTACAAGGCCAAGAAGCCCGTGC |
| | AGCTGCCCGGCGCCTACAACGTCAACATCAAGTTGGACATCACCTCCCACAACGAGG |
| | ACTACACCATCGTGGAACAGTACGAACGCGCCGAGGGCCGCCACTCCACCGGCGGCA |
| | TGGACGAGCTGTACAAGTGA |
| Construct 19- | TGTACAAAAAAGCAGGCTTTAAAGGAACCAATTCAGTCGACTGGATCCGGTACCAAG |
| U6p_20nt1_16bbarcode_ | GTCGGGCAGGAAGAGGGCCTATTTCCCATGATTCCTTCATATTTGCATATACGATACA |
| library | AGGCTGTTAGAGAGATAATTAGAATTAATTTGACTGTAAACACAAAGATATTAGTAC |
| SEQ ID NO: 26 | AAAATACGTGACGTAGAAAGTAATAATTTCTTGGGTAGTTTGCAGTTTTAAAATTATG |
| | TTTTAAAATGGACTATCATATGCTTACCGTAACTTGAAAGTATTTCGATTTCTTGGCTT |
| | TATATATCTTGTGGAAAGGACGAAACACCGTAAGTCGGAGTACTGTCCTGGGTTAGA |
| | GCTAGAAATAGCAAGTTAACCTAAGGCTAGTCCGTTATCAACTTGAAAAAGTGGCAC |
| | CGAGTCGGTGCTTTTTTGCAAGCAGNNNNNNNNNNNNNNNNTCTAGA |
| Construct 20- | TGTACAAAAAAGCAGGCTTTAAAGGAACCAATTCAGTCGACTGGATCCGGTACCAAG |
| U6p_20nt2_16bbarcode_ | GTCGGGCAGGAAGAGGGCCTATTTCCCATGATTCCTTCATATTTGCATATACGATACA |
| library | AGGCTGTTAGAGAGATAATTAGAATTAATTTGACTGTAAACACAAAGATATTAGTAC |
| SEQ ID NO: 27 | AAAATACGTGACGTAGAAAGTAATAATTTCTTGGGTAGTTTGCAGTTTTAAAATTATG |
| | TTTTAAAATGGACTATCATATGCTTACCGTAACTTGAAAGTATTTCGATTTCTTGGCTT |
| | TATATATCTTGTGGAAAGGACGAAACACCGGTGGCTTTACCAACAGTACGGGTTAGA |
| | GCTAGAAATAGCAAGTTAACCTAAGGCTAGTCCGTTATCAACTTGAAAAAGTGGCAC |
| | CGAGTCGGTGCTTTTTTGCAAGCAGNNNNNNNNNNNNNNNNTCTAGA |
| Construct 21- | TGTACAAAAAAGCAGGCTTTAAAGGAACCAATTCAGTCGACTGGATCCGGTACCAAG |
| U6p_30nt1_16bbarcode_ | GTCGGGCAGGAAGAGGGCCTATTTCCCATGATTCCTTCATATTTGCATATACGATACA |
| library | AGGCTGTTAGAGAGATAATTAGAATTAATTTGACTGTAAACACAAAGATATTAGTAC |
| SEQ ID NO: 28 | AAAATACGTGACGTAGAAAGTAATAATTTCTTGGGTAGTTTGCAGTTTTAAAATTATG |
| | TTTTAAAATGGACTATCATATGCTTACCGTAACTTGAAAGTATTTCGATTTCTTGGCTT |
| | TATATATCTTGTGGAAAGGACGAAACACCGATTCATCTCATCTATCAGAAAATAAATA |
| | AAGGGTTAGAGCTAGAAATAGCAAGTTAACCTAAGGCTAGTCCGTTATCAACTTGAA |
| | AAAGTGGCACCGAGTCGGTGCTTTTTTGCAAGCAGNNNNNNNNNNNNNNNNTCTAGA |
| Construct 22- | TGTACAAAAAAGCAGGCTTTAAAGGAACCAATTCAGTCGACTGGATCCGGTACCAAG |
| U6p_30nt2_16bbarcode_ | GTCGGGCAGGAAGAGGGCCTATTTCCCATGATTCCTTCATATTTGCATATACGATACA |
| library | AGGCTGTTAGAGAGATAATTAGAATTAATTTGACTGTAAACACAAAGATATTAGTAC |
| SEQ ID NO: 29 | AAAATACGTGACGTAGAAAGTAATAATTTCTTGGGTAGTTTGCAGTTTTAAAATTATG |
| | TTTTAAAATGGACTATCATATGCTTACCGTAACTTGAAAGTATTTCGATTTCTTGGCTT |
| | TATATATCTTGTGGAAAGGACGAAACACCGCAAATACCTCACACACTCCCAATACATG |
| | AAGGGTTAGAGCTAGAAATAGCAAGTTAACCTAAGGCTAGTCCGTTATCAACTTGAA |
| | AAAGTGGCACCGAGTCGGTGCTTTTTTGCAAGCAGNNNNNNNNNNNNNNNNTCTAGA |
| Construct 23- | TGTACAAAAAAGCAGGCTTTAAAGGAACCAATTCAGTCGACTGGATCCGGTACCAAG |
| U6p_40nt1_16bbarcode_ | GTCGGGCAGGAAGAGGGCCTATTTCCCATGATTCCTTCATATTTGCATATACGATACA |
| library | AGGCTGTTAGAGAGATAATTAGAATTAATTTGACTGTAAACACAAAGATATTAGTAC |
| SEQ ID NO: 30 | AAAATACGTGACGTAGAAAGTAATAATTTCTTGGGTAGTTTGCAGTTTTAAAATTATG |
| | TTTTAAAATGGACTATCATATGCTTACCGTAACTTGAAAGTATTTCGATTTCTTGGCTT |
| | TATATATCTTGTGGAAAGGACGAAACACCGTCACCACATTATATCAATTACTTCTTAA |
| | ATCACACAATCAGGGTTAGAGCTAGAAATAGCAAGTTAACCTAAGGCTAGTCCGTTA |
| | TCAACTTGAAAAAGTGGCACCGAGTCGGTGCTTTTTTGCAAGCAGNNNNNNNNNNNN |
| | NNNNTCTAGA |
| Construct 24- | TGTACAAAAAAGCAGGCTTTAAAGGAACCAATTCAGTCGACTGGATCCGGTACCAAG |
| U6p_40nt2_16bbarcode_ | GTCGGGCAGGAAGAGGGCCTATTTCCCATGATTCCTTCATATTTGCATATACGATACA |
| library | AGGCTGTTAGAGAGATAATTAGAATTAATTTGACTGTAAACACAAAGATATTAGTAC |
| SEQ ID NO: 31 | AAAATACGTGACGTAGAAAGTAATAATTTCTTGGGTAGTTTGCAGTTTTAAAATTATG |
| | TTTTAAAATGGACTATCATATGCTTACCGTAACTTGAAAGTATTTCGATTTCTTGGCTT |
| | TATATATCTTGTGGAAAGGACGAAACACCGTTACAAAATACAATTAATTAAAACTAC |
| | ATCAAAACACACAGGGTTAGAGCTAGAAATAGCAAGTTAACCTAAGGCTAGTCCGTT |
| | ATCAACTTGAAAAAGTGGCACCGAGTCGGTGCTTTTTTGCAAGCAGNNNNNNNNNNN |
| | NNNNNTCTAGA |
| Construct 25- | TGTACAAAAAAGCAGGCTTTAAAGGAACCAATTCAGTCGACTGGATCCGGTACCAAG |
| U6p_20nt_sgRNA_target | GTCGGGCAGGAAGAGGGCCTATTTCCCATGATTCCTTCATATTTGCATATACGATACA |
| SEQ ID NO: 32 | AGGCTGTTAGAGAGATAATTAGAATTAATTTGACTGTAAACACAAAGATATTAGTAC |
| | AAAATACGTGACGTAGAAAGTAATAATTTCTTGGGTAGTTTGCAGTTTTAAAATTATG |
| | TTTTAAAATGGACTATCATATGCTTACCGTAACTTGAAAGTATTTCGATTTCTTGGCTT |
| | TATATATCTTGTGGAAAGGACGAAACACCGTAAGTCGGAGTACTGTCCTGTTTTAGAG |
| | CTAGAAATAGCAAGTTAAAATAAGGCTAGTCCGTTATCAACTTGAAAAAGTGGCACC |
| | GAGTCGGTGCTTTTTTTCTAGAATCGCTAAACTGCGTCGCGGAGCCTTATGGCATAGG |
| | TCGTCCGCGGAGCATTCCGGTAACGCTTATGGTCCATAGCACATTCATCGCATCCGGG |
| | CGTGCGCTCTATTTGACGATCCCTTGGCGCAGAGGTGCTGGCCACGTGCTAAATTAAA |
| | GCGGCTGCACTACTGTAAGGTCCGTCGGCCGTCGATCCACCGATTCGCGTCGTGCGTA |
| | AGTCGGAGTACTGTCCTGGGGCTAGC |
| Construct 26- | TGTACAAAAAAGCAGGCTTTAAAGGAACCAATTCAGTCGACTGGATCCGGTACCAAG |
| U6p_30nt_sgRNA_target | GTCGGGCAGGAAGAGGGCCTATTTCCCATGATTCCTTCATATTTGCATATACGATACA |
| SEQ ID NO: 33 | AGGCTGTTAGAGAGATAATTAGAATTAATTTGACTGTAAACACAAAGATATTAGTAC |
| | AAAATACGTGACGTAGAAAGTAATAATTTCTTGGGTAGTTTGCAGTTTTAAAATTATG |
| | TTTTAAAATGGACTATCATATGCTTACCGTAACTTGAAAGTATTTCGATTTCTTGGCTT |
| | TATATATCTTGTGGAAAGGACGAAACACCGCAAATACCTCACACACTCCCAATACATG |
| | AAGTTTTAGAGCTAGAAATAGCAAGTTAAAATAAGGCTAGTCCGTTATCAACTTGAA |
| | AAAGTGGCACCGAGTCGGTGCTTTTTTTCTAGAATCGCTAAACTGCGTCGCGGAGCCT |
| | TATGGCATAGTCGTCCGCGGAGCATTCCGGTAACGCTTATGGTCCATAGCACATTCAT |
| | CGCATCCGGGCGTGCGCTCTATTTGACGATCCCTTGGCGCAGAGGGCTGGCCAGTGCT |
| | AAATTAAAGCGGCTGCACTACTGTAAGGTCCGTCGGCCGTCGATCCACCGATTCGCGT |
| | CGTGCGCAAATACCTCACACACTCCCAATACATGAAGGGGCTAGC |
| Construct 27- | TGTACAAAAAAGCAGGCTTTAAAGGAACCAATTCAGTCGACTGGATCCGGTACCAAG |
| U6p_40nt_sgRNA_target | GTCGGGCAGGAAGAGGGCCTATTTCCCATGATTCCTTCATATTTGCATATACGATACA |
| SEQ ID NO: 34 | AGGCTGTTAGAGAGATAATTAGAATTAATTTGACTGTAAACACAAAGATATTAGTAC |
| | AAAATACGTGACGTAGAAAGTAATAATTTCTTGGGTAGTTTGCAGTTTTAAAATTATG |
| | TTTTAAAATGGACTATCATATGCTTACCGTAACTTGAAAGTATTTCGATTTCTTGGCTT |
| | TATATATCTTGTGGAAAGGACGAAACACCGTCACCACATTATATCAATTACTTCTTAA |
| | ATCACACAATCAGTTTTAGAGCTAGAAATAGCAAGTTAAAATAAGGCTAGTCCGTTAT |
| | CAACTTGAAAAAGTGGCACCGAGTCGGTGCTTTTTTTCTAGAATCGCTAAACTGCGTC |
| | GCGGAGCCTTATGGCATAGTCGTCCGCGGAGCATTCCGGTAACGCTTATGGTCCATAG |
| | CACATTCATCGCATCCGGGCGTGCGCTCTATTTGACGATCCCTTGGCGCAGAGGTGCT |
| | GGCCACGTGCTAAATTAAAGCGGCTGCACTACTGTAAGGTCCGTCGGCCGTCGATCCA |
| | CCGATTCGCGTCGTGCGTCACCACATTATATCAATTACTTCTTAAATCACACAATCAG |
| | GGGCTAGC |
| Construct 28- | GCGCCGGGTTTTGGCGCCTCCCGCGGGCGCCCCCCTCCTCACGGCGAGCGCTGCCACG |
| hUBCp_TetR_p2a_LacI_p2a_ | TCAGACGAAGGGCGCAGGAGCGTTCCTCATCCTTCCGCCCGGACGCTCAGGACAGCG |
| ZeoR | GCTCGCTGCTCATAAGACTCGGCCTTAGAACCCCAGTATCAGCAGAAGGACATTTTAG |
| SEQ ID NO: 35 | GACGGGACTTGGGTGACTCTAGGGCACTGGTTTTCTTTCCAGAGAGCGGAACAGGCG |
| | AGGAAAAGTAGTCCCTTCTCGGCGATTCTGCGGAGGGATCTCCGTGGGGCGGTGAAC |
| | GCCGATGATTATATAAGGACGCGCCGGGTGTGGCACAGCTAGTTCCGTCGCAGCCGG |
| | GATTTGGGTCGCGGTTCTTGTTTGTCGGATCGCTGTGATCGTCACTTGGTGAGTTGCGGG |
| | CTGCTGGGCTGGCCGGGGCTTTCGTGGCCGCCGGGCCGCTCGGTGGGACGGAAGCGT |
| | GTGGAGAGACCGCCAAGGGCTGTAGTCTGGGTCCGCGAGCAAGGTTGCCCTTGAACTG |
| | GGGGTTGGGGGGAGCGCACAAAATGGCGGCTGTTCCCGAGTCTTGAATGGAAGACGC |
| | TTGTAAGGCGGGCTGTGAGGTCGTTGAAACAAGGTGGGGGGCATGGTGGGCGGCAAG |
| | AACCCTAAGGTCTTGAGGCCTTCGCTAATGCGGGAAAGCTCTTATTCGGGTGAGATGGG |
| | CTGGGGCACCATCTGGGGACCCTGACGTGAAGTTTGTCACTGACTGGAGAACTCGGGT |
| | TTGTCGTCTGGTTGCGGGGGCGGCAGTTATGCGGTGCCGTTGGGCAGTGCACCCGTAC |
| | CTTTGGGAGCGCGCGCCTCGTCGTGTCGTGACGTCACCCGTTCTGTTGGCTTATAATGC |
| | AGGGTGGGGCCACCrGCCGGTAGGTGTGCGGTAGGCTTTTCTCCGTCGCAGGACGCA |
| | GGGTTCGGGCCTAGGGTAGGCTCTCCTGAATCGACAGGCTTCCGGACCTCTGGTGAGG |
| | GGAGGGATAAGTGAGGCGTCAGTTTCTTTGGTCGGTTTTATGTACCTATCTTCTTAAGT |
| | AGCTGAAGCTCCGGTTTTGAACTATGCGCTCGGGGTTGGCGAGTGTGTTTTGTGAAGT |
| | TTTTTAGGCACCTTTTGAAATGTAATCATTTGGGTCAATATGTAATTTTCAGTGTTAGA |
| | CTAGTAAATTGTCCGCTAAATTCTGGCCGTTTTTGGCTTTTTTGTTAGACAGGATCCCC |
| | GGGTACCGGTCGCCACCATGTTTTCGGTTGGACAAATCTAAAGTAATCAACTTTTGCACT |
| | GGAATTGCTGAACGAGGTAGGCATAGAGGGCCTCACAACGAGGAAGCTGGCCCAAA |
| | AGCTGGGCGTCGAACAGCCAACCCTGTACTGGCACGTCAAGAATAAAAGGGCTCTCC |
| | TGGACGCGCTGGCATTTGAGTTGCTCGACAGACACCATACACACTTTTGCCCCCTTGT |
| | AGGGGAATCCTGGCAGGACTTCCTGCGAAACAATGCCAAGTCATTTAGATGCGCTCT |
| | CTGTCTCATCGGGACGGTGCTAAGGTGCATCTGGGTACAAGACCCACGGAAAAGCAG |
| | TATGAGACACTGGAAAATCAACTGGCCTTTTTGTGTCAGCAGGGCTTCTCTCTCGAAA |
| | ACGCGCTTTACGCGCTGTCAGCCGTGGGTCATTTTACCCTGGGCTGCGTGCTGGAGGA |
| | CCAGGAGCATCAAGTGGCTAAGGAGGAACGGGAAACCCCTACCACCGACTCTATGCC |
| | ACCTCTCTTGCGGCAGGCAATTGAGTTGTTCGACCACCAGGGTGCCGAGCCGGCCTTC |
| | CTGTTCGGCTTGGAGCTTATCATCTGCGGCCTGGAGAAGCAGCTGAAGTGTGAGAGTG |
| | GAAGTCGTACGGGAAGCGGAGCTACTAACTTCAGCCTGCTGAAGCAGGCTGGAGACG |
| | TGGAGGAGAACCCTGGACCTAAACCAGTAACATTGTATGATGTCGCAGAGTATGCCC |
| | GTGTCTCTTATCAGACTGTTTCCAGAGTGGTGAACCAGGCCAGCCATGTTTCTGCCAA |
| | AACCAGGGAAAAAGTGGAAGCAGCCATGGCAGAGCTGAATTACATTCCCAACAGAGT |
| | GGCACAACAACTGGCAGGCAAACAGAGCTTGCTGATTGGAGTTGCCACCTCCAGTCT |
| | GGCCCTGCATGCACCATCTCAAATTGTGGCAGCCATTAAATCTAGAGCTGATCAACTG |
| | GGAGCCTCTGTGGTGGTGTCAATGGTAGAAAGAAGTGGAGTTGAAGCCTGTAAAGCT |
| | GCTGTGCACAATCTTCTGGCACAAAGAGTCAGTGGGCTGATCATTAACTATCCACTGG |
| | ATGACCAGGATGCCATTGCTGTGGAAGCTGCCTGCACTAATGTTCCAGCACTCTTTCT |
| | TGATGTCTCTGACCAGACACCCATCAACAGTATTATTTTCTCCCATGAAGATGGTACA |
| | AGACTGGGTGTGGAGCATCTGGTTGCATTGGGACACCAGCAAATTGCACTGCTTGCGG |
| | GCCCACTCAGTTCTGTCTCAGCAAGGCTGAGACTGGCTGGCTGGCATAAATATCTCAC |
| | TAGGAATCAAATTCAGCCAATAGCTGAAAGAGAAGGGGACTGGAGTGCCATGTCTGG |
| | GTTTTAACAAACCATGCAAATGCTGAATGAGGGCATTTTGTTCCCACTGCAATGCTGGT |
| | GCCAATGATCAGATGGCACTGGGTGCAATGAGAGCCATTACTGAGTCTGGGCTGAGA |
| | GTTGGTGCAGATATCTCGGTAGTGGGATACGACGATACCGAAGACAGCTCATGTTATA |
| | TCCCGCCGTTAACCACCATCAAACAGGATTTCGCCTGCTGGGGCAAACCAGCGTGGA |
| | CCGCTTGCTGCAACTCTCTCAGGGCCAGGCGGTGAAGGGCAATCAGCTGTTGCCAGTC |
| | TCACTGGTGAAGAGAAAAACCACCCTGGCACCCAATACACAAACTGCCTCTCCCCGG |
| | GCATTGGCTGATTCACTCATGCAGCTAGCAAGACAGGTTTCCAGACTGGAAAGTGGG |
| | CAGAGCAGCCTGAGGCCTCCTAAGAAGAAGAGGAAGGTTGGCTCTGGTGCAACCAAT |
| | TTCTCTCTTCTTAAACAAGCCGGTGATGTGGAGGAGAACCCCGGACCCGCCAAGTTGA |
| | CCAGTGCCGTTCCGGTGCTCACCGCGCGCGACGTCGCCGGAGCGGTCGAGTTCTGGAC |
| | CGACCGGCTCGGGTTCTCCCGGGACTTCGTGGAGGACGACTTCGCCGGTGTGGTCCGG |
| | GACGACGTGACCCTGTTCATCAGCGCGGTCCAGGACCAGGTGGTGCCGGACAACACC |
| | CTGGCCTGGGTGTGGGTGCGCGGCCTGGACGAGCTGTACGCCGAGTGGTCGGAGGTC |
| | GTGTCCACGAACTTCCGGGACGCCTCCGGGCCGGCCATGACCGAGATCGGCGAGCAG |
| | CCGTGGGGGCGGGAGTTCGCCCTGCGCGACCCGGCCGGCAACTGCGTGCACTTCGTG |
| | GCCGAGGAGCAGGACTGA |
| Construct 29- | GAATCCTATGCTTCGAACGCTGACGTCATCAACCCGCTCCAAGGAATCGCGGGCCCAG |
| 1xTetO_H1p_20nt3_stgRNA | TGTCACTAGGCGGGAACACCCAGCGCGCGTGCGCCCTGGCAGGAAGATGGCTGTGAG |
| SEQ ID NO: 36 | GGACAGGGGAGTGGCGCCCTGCAATATTTGCATGTCGCTATGTGTTCTGGGAAATCAC |
| | CATAAACGTGAAATGTCTTTGGATTTGGGAATCTTATAAGTCCCTATCAGTGATAGAG |
| | ATCCCAAGTCGCGTGTAGCGAAGCAGGGTTAGAGCTAGAAATAGCAAGTTAACCTAA |
| | GGCTAGTCCGTTATCAACTTGAAAAAGTGGCACCGAGTCGGTGCTTTTTT |
| Construct 30- | TGTACAAAAAAGCAGGCTTTAAAGGAACCAATTCAGTCGACTGGATCCGGTACCAAG |
| 3xLacO_U6p_20nt3_stgRNA | GTCGGGCAGGAAGAGGGCCTATTTCCCATGATTCCTTCATATTTGCATATACGATACA |
| SEQ ID NO: 37 | AGGCTGTTAGAGAGATAATTAGAATTAATTTGACTGTAAACACAAAGATATTAGTAC |
| | AAAAAATTGTGAGCGGATAACAATTATTTCTTGGGTAGTTTGCAGTTTTAAAATTATG |
| | TTTTAAAATGGACTATCATATGCTTACCGTAACTTGAAAGTAATTGTGAGCGCTCACA |
| | ATTATATATCTTGTGGAAAGGACGAAACACCGAGTCGCGTGTAGCGAAGCAGGGTTA |
| | GAGCTAGAAATAGCAAGTTAACCTAAGGCTAGTCCGTTATCAACTTGAAAAAGTGGC |
| | ACCGAGTCGGTGCTTTTTTCTAGACCCAGCAATTGTGAGCGCTCACAATT |
| Construct 31- | GAATCCTATGCTTCGAACGCTCACGTCATCAACCCGCTCCAAGGAATCGCGGGCCCAG |
| 1xTetO_H1p_20nt2_stgRNA_3x | TGTCACTAGGCGGGAACACCCAGCGCGCGTGCGCCCTGGCAGGAAGATGGCTGTGAG |
| LacO_U6p_20nt3_stgRNA | GGACAGGGGAGTGGCGCCCTGCAATATTTGCATGTCGCTATGTGTTCTGGGAAATCAC |
| SEQ ID NO: 38 | CATAAACGTGAAATGTCTTTGGATTTGGGAATCTTATAAGTCCCTATCAGTGATAGAG |
| | ATCCCAGTGGCTTTACCAACAGTACGGGTTAGAGCTAGAAATAGCAAGTTAACCTAA |
| | GGCTAGTCCGTTATCAACTTGAAAAAGTGGCACCGAGTCGGTGCTTTTTTTCACGAGG |
| | CGGACACTGATTGACACGGTTTGCTAGCTGTACAAAAAAGCAGGCTTTAAAGGAACC |
| | AATTCAGTCGACTGGATCCGGTACCAAGGTCGGGCAGGAAGAGGGCCTATTTCCCAT |
| | GATTCCTTCATATTTGCATATACGATACAAGGCTGTTAGAGAGATAATTAGAATTAAT |
| | TTGACTGTAAACACAAAGATATTAGTACAAAAAATTGTGAGCGGATAACAATTATTTC |
| | TTGGGTAGTTTGCAGTTTTAAAATTATGTTTTAAAATGGACTATCATATGCTTACCGTA |
| | ACTTGAAAGTAATTGTGAGCGCTCACAATTATATATCTTGTGGAAAGGACGAAACACC |
| | GAGTCGCGTGTAGCGAAGCAGGGTTAGAGCTAGAAATAGCAAGTTAACCTAAGGCTA |
| | GTCCGTTATCAACTTGAAAAAGTGGCACCGAGTCGGTGCTTTTTTCTAGACCCAGCAA |
| | TTGTGAGCGCTCACAATT |
| Construct 32- | GGGGACTTTCCGGGAATTTCCGGGGACTTTCCGGGAATTTCCGGGAATTTCCGGGGAC |
| NFKBRp_mKate2_2xNLS_p2a- | TTTCCGGGAATTTCCGGGGACTTTCCGGGAATTTCCAGATCTGGCCTCGGCGGCCAAG |
| puroR | CTTGCTAGCGGGGGGCTATAAAAGGGGGTGGGGGCGTTCGTCCTCACTCTAGTTCTGC |
| SEQ ID NO: 39 | GATCTAAGTAAGCTTGGCATTACCGGTCGCCAACGCGTGCCACCATGGTGAGCGAGCT |
| | GATTAAGGAGAACATGCACATGAAGCTGTACATGGAGGGCACCGTGAACAACCACCA |
| | CTTCAAGTGCACATCCGAGGGCGAAGGCAAGCCCTACGAGGGCACCCAGACCATGAG |
| | AATCAAGGCGGTCGAGGGCGGCCCTCTCCCCTTCGCCTTCGACATCCTGGCTACCAGC |
| | TTCATGTACGGCAGCAAAACCTTCATCAACCACACCCAGGGCATCCCCGACTTCTTTA |
| | AGCAGTCCTTCCCCGAGGGCTTCACATGGGAGAGAGTCACCACATACGAAGATGGGG |
| | GCGTGCTGACCGCTACCCAGGACACCAGCCTCCAGGACGGCTGCCTCATCTACAACGT |
| | CAAGATCAGAGGGGTGAACTTCCCATCCAACGGCCCTGTGATGCAGAAGAAAACACT |
| | CGGCTGGGAGGCCTCCACCGAGACACTGTACCCCGCTGACGGCGGCCTGGAAGGCAG |
| | AGCCGACATGGCCCTGAAGCTCGTGGGCGGGGGCCACCTGATCTGCAACCTTAAGAC |
| | CACATACAGATCCAAGAAACCCGCTAAGAACCTCAAGATGCCCGGCGTCTACTATGT |
| | GGACAGGAGACTGGAAAGAATCAAGGAGGCCGACAAAGAGACATACGTCGAGCAGC |
| | ACGAGGTGGCTGTGGCCAGATACTGCGACCTCCCTAGCAAACTGGGGCACAAACTTA |
| | ATTCCGGATCCCCAAAGAAGAAAAGGAAGGTTGAAGACCCCAAGAAAAAGAGGAAG |
| | GTGATAAGCGCTGGAAGCGGAGCTACTAACTTCAGCCTGCTGAAGCAGGCTGGAGAC |
| | GTGGAGGAGAACCCTGGACCTACCGAGTACAAGCCCACGGTGCGCCTCGCCACCCGC |
| | GACGACGTCCCCCGGGCCGTACGCACCCTCGCCGCCGCGTTCGCCGACTACCCCGCCA |
| | CGCGCCACACCGTCGACCCGGACCGCCACATCGAGCGGGTCACCGAGCTGCAAGAAC |
| | TCTTCCTCACGCGCGTCGGGCTCGACATCGGCAAGGTGTGGGTCGCGGACGACGGCG |
| | CCGCGGTGGCGGTCTGGACCACGCCGGAGAGCGTCGAAGCGGGGGCGGTGTTCGCCG |
| | AGATCGGCCCGCGCATGGCCGAGTTGAGCGGTTCCCGGCTGGCCGCGCAGCAACAGA |
| | TGGAAGGCCTCCTGGCGCCGCACCGGCCCAAGGAGCCCGCGTGGTTCCTGGCCACCG |
| | TCGGCGTCTCGCCCGACCACCAGGGCAAGGGTCTGGGCAGCGCCGTCGTGCTCCCCG |
| | GAGTGGAGGCGGCCGAGCGCGCCGGGGTGCCCGCCTTCCTGGAGACATCCGCGCCCC |
| | GCAACCTCCCCTTCTACGAGCGGCTCGGCTTCACCGTCACCGCCGACGTCGAGGTGCC |
| | CGAAGGACCGCGCACCTGGTGCATGACCCGCAAGCCCGGTGCCTGA |
| Construct 33 - | GGGGACTTTCCGGGAATTTCCGGGGACTTTCCGGGAATTTCCGGGAATTTCCGGGGAC |
| NFKBRp_Cas9_3xNLS_p2a- | TTTCCGGGAATTTCCGGGGACTTTCCGGGAATTTCCAGATCTGGCCTCGGCGGCCAAG |
| puroR | CTTGCTAGCGGGGGGCTATAAAAGGGGGTGGGGGCGTTCGTCCTCACTCTAGTTCTGC |
| SEQ ID NO: 40 | GATCTAAGTAAGCTTGGCATTACCGGTCGCCAACGCGTGCCACCATGGACAAGAAGT |
| | ACAGCATCGGCCTGGACATCGGCACCAACTCTGTGGGCTGGGCCGTGATCACCGACG |
| | AGTACAAGGTGCCCAGCAAGAAATTCAAGGTGCTGGGCAACACCGACCGGCACAGCA |
| | TCAAGAAGAACCTGATCGGAGCCCTGCTGTTCGACAGCGGCGAAACAGCCGAGGCCA |
| | CCCGGCTGAAGAGAACCGCCAGAAGAAGATACACCAGACGGAAGAACCGGATCTGC |
| | TATCTGCAAGAGATCTTCAGCAACGAGATGGCCAAGGTGGACGACAGCTTCTTCCAC |
| | AGACTGGAAGAGTCCTTCCTGGTGGAAGAGGATAAGAAGCACGAGCGGCACCCCATC |
| | TTCGGCAACATCGTGGACGAGGTGGCCTACCACGAGAAGTACCCCACCATCTACCAC |
| | CTGAGAAAGAAACTGGTGGACAGCACCGACAAGGCCGACCTGCGGCTGATCTATCTG |
| | GCCCTGGCCCACATGATCAAGTTCCGGGGCCACTTCCTGATCGAGGGCGACCTGAACC |
| | CCGACAACAGCGACGTGGACAAGCTGTTCATCCAGCTGGTGCAGACCTACAACCAGC |
| | TGTTCGAGGAAAACCCCATCAACGCCAGCGGCGTGGACGCCAAGGCCATCCTGTCTG |
| | CCAGACTGAGCAAGAGCAGACGGCTGGAAAATCTGATCGCCCAGCTGCCCGGCGAGA |
| | AGAAGAATGGCCTGTTCGGAAACCTGATTGCCCTGAGCCTGGGCCTGACCCCCAACTT |
| | CAAGAGCAACTTCGACCTGGCCGAGGATGCCAAACTGCAGCTGAGCAAGGACACCTA |
| | CGACGACGACCTGGACAACCTGCTGGCCCAGATCGGCGACCAGTACGCCGACCTGTT |
| | TCTGGCCGCCAAGAACCTGTCCGACGCCATCCTGCTGAGCGACATCCTGAGAGTGAAC |
| | ACCGAGATCACCAAGGCCCCCCTGAGCGCCTCTATGATCAAGAGATACGACGAGCAC |
| | CACCAGGACCTGACCCTGCTGAAAGCTCTCGTGCGGCAGCAGCTGCCTGAGAAGTAC |
| | AAAGAGATTTTCTTCGACCAGAGCAAGAACGGCTACGCCGGCTACATTGACGGCGGA |
| | GCCAGCCAGGAAGAGTTCTACAAGTTCATCAAGCCCATCCTGGAAAAGATGGACGGC |
| | ACCGAGGAACTGCTCGTGAAGCTGAACAGAGAGGACCTGCTGCGGAAGCAGCGGACC |
| | TTCGACAACGGCAGCATCCCCCACCAGATCCACCTGGGAGAGCTGCACGCCATTCTGC |
| | GGCGGCAGGAAGATTTTTACCCATTCCTGAAGGACAACCGGGAAAAGATCGAGAAGA |
| | TCCTGACCTTCCGCATCCCCTACTACGTGGGCCCTCTGGCCAGGGGAAACAGCAGATT |
| | CGCCTGGATGACCAGAAAGAGCGAGGAAACCATCACCCCCTGGAACTTCGAGGAAGr |
| | GGTGGACAAGGGCGCTTCCGCCCAGAGCTTCATCGAGCGGATGACCAACTTCGATAA |
| | GAACCTGCCCAACGAGAAGGTGCTGCCCAAGCACAGCCTGCTGTACGAGrACTTCAC |
| | CGTGTATAACGAGCTGACCAAAGTGAAATACGTGACCGAGGGAATGAGAAAGCCCGC |
| | CTTCCTGAGCGGCGAGCAGAAAAAGGCCATCGTGGACCTGCTGTTCAAGACCAACCG |
| | GAAAGTGACCGTGAAGCAGCTGAAAGAGGACTACTTCAAGAAAATCGAGTGCTTCGA |
| | CTCCGTGGAAATCTCCGGCGTGGAAGATCGGTTCAACGCCTCCCTGGGCACATACCAC |
| | GATCTGCTGAAAATTATCAAGGACAAGGACTTCCTGGACAATGAGGAAAACGAGGAC |
| | ATTCTGGAAGATATCGTGCTGACCCTGACACTGTTTGAGGACAGAGAGATGATCGAG |
| | GAACGGCTGAAAACCTATGCCCACCTGTTCGACGACAAAGTGATGAAGCAGCTGAAG |
| | CGGCGGAGATACACCGGCTGGGGCAGGCTGAGCCGGAAGCTGATCAACGGCATCCGG |
| | GACAAGCAGTCCGGCAAGACAATCCTGGATTTCCTGAAGTCCGACGGCTTCGCCAAC |
| | AGAAACTTCATGCAGCTGATCCACGACGACAGCCTGACCTTTAAAGAGGACATCCAG |
| | AAAGCCCAGGTGTCCGGCCAGGGCGATAGCCTGCACGAGCACATTGCCAATCTGGCC |
| | GGCAGCCCCGCCATTAAGAAGGGCATCCTGCAGACAGTGAAGGTGGTGGACGAGCTC |
| | GTGAAAGTGATGGGCCGGCACAAGCCCGAGAACATCGTGATCGAAATGGCCAGAGA |
| | GAACCAGACCACCCAGAAGGGACAGAAGAACAGCCGCGAGAGAATGAAGCGGATCG |
| | AAGAGGGCATCAAAGAGCTGGGCAGCCAGATCCTGAAAGAACACCCCGTGGAAAAC |
| | ACCCAGCTGCAGAACGAGAAGCTGTACCTGTACTACCTGCAGAATGGGCGGGATATG |
| | TACGTGGACCAGGAACTGGACATCAACCGGCTGTCCGACTACGATGTGGACCATATC |
| | GTGCCTCAGAGCTTTCTGAAGGACGACTCCATCGACAACAAGGTGCTGACCAGAAGC |
| | GACAAGAACCGGGGCAAGAGCGACAACGTGCCCTCCGAAGAGGTCGTGAAGAAGAT |
| | GAAGAACTACTGGCGGCAGCTGCTGAACGCCAAGCTGATTACCCAGAGAAAGTTCGA |
| | CAATCTGACCAAGGCCGAGAGAGGCGGCCTGAGCGAACTGGATAAGGCCGGCTTCAT |
| | CAAGAGACAGCTGGTGGAAACCCGGCAGATCACAAAGCACGTGGCACAGATCCTGGA |
| | CTCCCGGATGAACACTAAGTACGACGAGAATGACAAGCTGATCCGGGAAGTGAAAGT |
| | GATCACCCTGAAGTCCAAGCTGGTGTCCGATTTCCGGAAGGATTTCCAGTTTTACAAA |
| | GTGCGCGAGATCAACAACTACCACCACGCCCACGACGCCTACCTGAACGCCGTCGTG |
| | GGAACCGCCCTGATCAAAAAGTACCCTAAGCTGGAAAGCGAGTTCGTGTACGGCGAC |
| | TACAAGGTGTACGACGTGCGGAAGATGATCGCCAAGAGCGAGCAGGAAATCGGCAA |
| | GGCTACCGCCAAGTACTTCTTCTACAGCAACATCATGAACTTTTTCAAGACCGAGATT |
| | ACCCTGGCCAACGGCGAGATCCGGAAGCGGCCTCTGATCGAGACAAACGGCGAAACC |
| | GGGGAGATCGTGTGGGATAAGGGCCGGGATTTTGCCACCGTGCGGAAAGTGCTGAGC |
| | ATGCCCCAAGTGAATATCGTGAAAAAGACCGAGGTGCAGACAGGCGGCTTCAGCAAA |
| | GAGTCTATCCTGCCCAAGAGGAACAGCGATAAGCTGATCGCCAGAAAGAAGGACTGG |
| | GACCCTAAGAAGTACGGCGGCTTCGACAGCCCCACCGTGGCCTATTCTGTGCTGGTGG |
| | TGGCCAAAGTGGAAAAGGGCAAGTCCAAGAAACTGAAGAGTGTGAAAGAGCTGCTG |
| | GGGATCACCATCATGGAAAGAAGCAGCTTCGAGAAGAATCCCATCGACTTTCTGGAA |
| | GCCAAGGGCTACAAAGAAGTGAAAAAGGACCTGATCATCAAGCTGCCTAAGTACTCC |
| | CTGTTCGAGCTGGAAAACGGCCGGAAGAGAATGCTGGCCTCTGCCGGCGAACTGCAG |
| | AAGGGAAACGAACTGGCCCTGCCCTCCAAATATGTGAACTTCCTGTACCTGGCCAGCC |
| | ACTATGAGAAGCTGAAGGGCTCCCCCGAGGATAATGAGCAGAAACAGCTGTTTGTGG |
| | AACAGCACAAGCACTACCTGGACGAGATCATCGAGCAGATCAGCGAGTTCTCCAAGA |
| | GAGTGATCCTGGCCGACGCTAATCTGGACAAAGTGCTGTCCGCCTACAACAAGCACC |
| | GGGATAAGCCCATCAGAGAGCAGGCCGAGAATATCATCCACCTGTTTACCCTGACCA |
| | ATCTGGGAGCCCCTGCCGCCTTCAAGTACTTTGACACCACCATCGACCGGAAGAGGTA |
| | CACCAGCACCAAAGAGGTGCTGGACGCCACCCTGATCCACCAGAGCATCACCGGCCT |
| | GTACGAGACACGGATCGACCTGTCTCAGCTGGGAGGCGACAAGCGTCCTGCTGCTAC |
| | TAAGAAAGCTGGTCAAGCTAAGAAAAAGAAAGCTAGCGGCAGCGGCGCCGGATCCC |
| | CAAAGAAGAAAAGGAAGGTTGAAGACCCCAAGAAAAAGAGGAAGGTGATAAGCGCT |
| | GGAAGCGGAGCTACTAACTTCAGCCTGCTGAAGCAGGCTGGAGACGTGGAGGAGAAC |
| | CCTGGACCTACCGAGTACAAGCCCACGGTGCGCCTCGCCACCCGCGACGACGTCCCCC |
| | GGGCCGTACGCACCCTCGCCGCCGCGTTCGCCGACTACCCCGCCACGCGCCACACCGT |
| | CGACCCGGACCGCCACATCGAGCGGGTCACCGAGCTGCAAGAACTCTTCCTCACGCG |
| | CGTCGGGCTCGACATCGGCAAGGTGTGGGTCGCGGACGACGGCGCCGCGGTGGCGGT |
| | CTGGACCACGCCGGAGAGCGTCGAAGCGGGGGCGGTGTTCGCCGAGATCGGCCCGCG |
| | CATGGCCGAGTTGAGCGGTTCCCGGCTGGCCGCGCAGCAACAGATGGAAGGCCTCCT |
| | GGCGCCGCACCGGCCCAAGGAGCCCGCGTGGTTCCTGGCCACCGTCGGCGTCTCGCCC |
| | GACCACCAGGGCAAGGGTCTGGGCAGCGCCGTCGTGCTCCCCGGAGTGGAGGCGGCC |
| | GAGCGCGCCGGGGTGCCCGCCTTCCTGGAGACATCCGCGCCCCGCAACCTCCCCTTCT |
| | ACGAGCGGCTCGGCTTCACCGTCACCGCCGACGTCGAGGTGCCCGAAGGACCGCGCA |
| | CCTGGTGCATGACCCGCAAGCCCGGTGCCTGA |

### REFERENCES, EACH OF WHICH IS INCORPORATED HEREIN

J. J. Collins, T. S. Gardner, C. R. Cantor, Construction of a genetic toggle switch in Escherichia coli. Nature. 403, 339-342 (2000).
J. W. Kotula et al., Programmable bacteria detect and record an environmental signal in the mammalian gut. Proc. Natl. Acad. Sci. U.S.A 111, 4838-43 (2014).
C. M. Ajo-franklin et al., Rational design of memory in eukaryotic cells service Rational design of memory in eukaryotic cells. Genes Dev. 21, 2271-2276 (2007).
D. R. Burrill et al., Synthetic memory circuits for tracking human cell fate. Genes Dev., 1486-1497 (2012).
L. Yang et al., Permanent genetic memory with >1-byte capacity. Nat Meth. 11, 1261-1266 (2014).
T. S. Ham, S. K. Lee, J. D. Keasling, A. P. Arkin, Design and construction of a double inversion recombination switch for heritable sequential genetic memory. PLoS One. 3, 1-9 (2008).
P. Siuti, J. Yazbek, T. K. Lu, Synthetic circuits integrating logic and memory in living cells. Nat. Biotechnol. 31, 448-452 (2013).
A. E. Friedland et al., Synthetic Gene Networks That Count. Science (80-.). 324, 1199-1202 (2009).
F. Farzadfard, T. K. Lu, Genomically encoded analog memory with precise in vivo DNA writing in living cell populations. Science (80-.). 346, 1256272 (2014).
L. Cong et al., Multiplex Genome Engineering Using CRISPR/Cas Systems. Science (80-.). 339, 819-823 (2013).
P. Mali et al., RNA-Guided Human Genome Engineering via Cas9. Science (80-.). 339, 823-826 (2013).
M. Jinek et al., RNA-programmed genome editing in human cells. Elife. 2, e00471-e00471 (2013).
S. H. Sternberg, S. Redding, M. Jinek, E. C. Greene, J. A. Doudna, DNA interrogation by the CRISPR RNA-guided endonuclease Cas9. Nature. 507, 62-67 (2014).
C. Anders, O. Niewoehner, A. Duerst, M. Jinek, Structural basis of PAM-dependent target DNA recognition by the Cas9 endonuclease. Nature. 513, 569-573 (2014).
B. Pardo, B. Gómez-González, A. Aguilera, DNA Repair in Mammalian Cells. Cell. Mol. Life Sci. 66, 1039-1056 (2009). M. T. Certo et al., Tracking genome engineering outcome at individual DNA breakpoints. Nat Meth. 8, 671-676 (2011).
B. J. Aubrey et al., An Inducible Lentiviral Guide RNA Platform Enables the Identification of Tumor-Essential Genes and Tumor-Promoting Mutations In Vivo. Cell Rep. 10, 1422-1432 (2015).
M. J. Herold, J. van den Brandt, J. Seibler, H. M. Reichardt, Inducible and reversible gene silencing by stable integration of an shRNA-encoding lentivirus in transgenic rats. Proc. Natl. Acad. Sci. U.S.A 105, 18507-18512 (2008).
Y. Paik et al., Toll-like receptor 4 mediates inflammatory signaling by bacterial lipopolysaccharide in human hepatic stellate cells. Hepatology. 37, 1043-1055 (2003).
D. J. Van Antwerp, S. J. Martin, T. Kafri, D. R. Green, I. M. Verma, Suppression of TNF-α-Induced Apoptosis by NF-κB. Science (80-.). 274, 787-789 (1996).
M. H. Bemelmans, D. J. Gouma, W. A. Buurman, LPS-induced sTNF-receptor release in vivo in a murine model. Investigation of the role of tumor necrosis factor, IL-1, leukemia inhibiting factor, and IFN-gamma. J. Immunol. 151, 5554-5562 (1993).
B. Bozkurt et al., Pathophysiologically Relevant Concentrations of Tumor Necrosis Factor-Promote Progressive Left Ventricular Dysfunction and Remodeling in Rats. Circulation. 97, 1382-1391 (1998).
B. Levine, J. Kalman, L. Mayer, H. M. Fillit, M. Packer, Elevated Circulating Levels of Tumor Necrosis Factor in Severe Chronic Heart Failure. N. Engl. J. Med. 323, 236-241 (1990).
T. L. Whiteside, The tumor microenvironment and its role in promoting tumor growth. Oncogene. 27, 5904-5912 (2008).
A. P. McMahon, P. W. Ingham, C. J. Tabin, B. T.-C. T. in D. Biology, Ed. (Academic Press, 2003; http://www.sciencedirect.com/science/article/pii/S0070215303530022), vol. Volume 53, pp. 1-114.
J. Taipale, P. A. Beachy, The Hedgehog and Wnt signalling pathways in cancer. Nature. 411, 349-354 (2001).
D. E. Cohen, D. Melton, Turning straw into gold: directing cell fate for regenerative medicine. Nat Rev Genet. 12, 243-252 (2011).
A. Wodarz, R. Nusse, MECHANISMS OF WNT SIGNALING IN DEVELOPMENT. Annu. Rev. Cell Dev. Biol. 14, 59-88 (1998).
A. S. Dhillon, S. Hagan, O. Rath, W. Kolch, MAP kinase signalling pathways in cancer. Oncogene. 26, 3279-3290.
M. Srivastava et al., An Inhibitor of Nonhomologous End-Joining Abrogates Double-Strand Break Repair and Impedes Cancer Progression. Cell. 151, 1474-1487 (2012).
J. J. J. Leahy et al., Identification of a highly potent and selective DNA-dependent protein kinase (DNA-PK) inhibitor (NU7441) by screening of chromenone libraries. Bioorg. Med. Chem. Lett. 14, 6083-6087 (2004).
M. Rouleau, A. Patel, M. J. Hendzel, S. H. Kaufmann, G. G. Poirier, PARP inhibition: PARP1 and beyond. Nat Rev Cancer. 10, 293-301 (2010).
B. P. Kleinstiver et al., Monomeric site-specific nucleases for genome editing. 109 (2012), doi:10.1073/pnas.1117984109.
M. Minczuk, M. A. Papworth, J. C. Miller, M. P. Murphy, A. Klug, Development of a single-chain, quasi-dimeric zinc-finger nuclease for the selective degradation of mutated human mitochondrial DNA. Nucleic Acids Res. 36, 3926-3938 (2008).
R. J. Klose, A. P. Bird, Genomic DNA methylation: the mark and its mediators. Trends Biochem. Sci. 31, 89-97 (2006).
M. L. Maeder et al., Targeted DNA demethylation and activation of endogenous genes using programmable TALE-TET1 fusion proteins. Nat Biotech. 31, 1137-1142 (2013).
A. C. Komor, Y. B. Kim, M. S. Packer, J. A. Zuris, D. R. Liu, Programmable editing of a target base in genomic DNA without double-stranded DNA cleavage. Nature. advance on (2016) (available at http://dx.doi.org/10.1038/nature17946).
J. H. Lee et al., Fluorescent in situ sequencing (FISSEQ) of RNA for gene expression profiling in intact cells and tissues. Nat. Protoc. 10, 442-458 (2015).
J. H. Lee et al., Highly multiplexed subcellular RNA sequencing in situ. Science. 343, 1360-1363 (2014).
L. Nissim, S. D. Perli, A. Fridkin, P. Perez-Pinera, T. K. Lu, Multiplexed and programmable regulation of gene networks with an integrated RNA and CRISPR/Cas toolkit in human cells. Mol. Cell. 54, 698-710 (2014).
C. Lois, E. J. Hong, S. Pease, E. J. Brown, D. Baltimore, Germline transmission and tissue-specific expression of transgenes delivered by lentiviral vectors. Science. 295, 868-872 (2002).
J. Zhang, K. Kobert, T. Flouri, A. Stamatakis, PEAR: a fast and accurate Illumina Paired-End reAd mergeR. Bioinformatics. 30, 614-620 (2014).
S. F. Altschul, B. W. Erickson, Optimal sequence alignment using affine gap costs. Bull. Math. Biol. 48, 603-616.
R. Lorenz et al., ViennaRNA Package 2.0. Algorithms Mol. Biol. 6, 1-14 (2011).
Cong L, et al. Science. 2013, 15; 339(6121):819-23.
Charpentier E, et al. Nature. 2013, 7; 495(7439):50-1.
Farzadfard F, et al. ACS Synth Biol. 2013, 18; 2(10):604-13.
Nissim L, et al. Mol Cell. 2014 May 22; 54(4):698-710.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. An engineered nucleic acid comprising a promoter operably linked to a nucleotide sequence encoding a guide ribonucleic acid (gRNA) that comprises a specificity determining sequence (SDS) and a protospacer adjacent motif (PAM).

2. The engineered nucleic acid of claim 1, wherein the PAM is a wild-type PAM.

3. The engineered nucleic acid of claim 1, wherein the PAM is downstream (3′) from the SDS.

4. The engineered nucleic acid of claim 1, wherein the PAM is adjacent to the SDS.

5. The engineered nucleic acid of claim 1, wherein the nucleotide sequence of the PAM is selected from the group consisting of NGG, NNGRR(T/N), NNNNGATT, NNAGAAW, and NAAAAC.

6. The engineered nucleic acid of claim 1, wherein the length of the SDS is 15 to 75 nucleotides or 20 nucleotides.

7. (canceled)

8. The engineered nucleic acid of claim 1, wherein the promoter is inducible.

9. A cell comprising the engineered nucleic acid of claim 1, optionally wherein the engineered nucleic acid is located in the genome of the cell.

10-11. (canceled)

12. An episomal vector comprising the engineered nucleic acid of claim 1.

13. A cell comprising the episomal vector of claim 12.

14. A method comprising introducing into a cell the engineered nucleic acid of claim 1.

15. (canceled)

16. A method comprising introducing into a cell the episomal vector of claim 12.

17. (canceled)

18. A self-contained analog memory device, comprising:
an engineered nucleic acid comprising an inducible promoter operably linked to a nucleotide sequence encoding a guide ribonucleic acid (gRNA) that comprises a specificity determining sequence (SDS) and a protospacer adjacent motif (PAM).

19. The device of claim 18, wherein the inducible promoter is regulated by a cell signaling protein, optionally wherein the cell signaling protein is a cytokine.

20. (canceled)

21. A cell comprising:
the device of claim 18; and
Cas9 nuclease.

22. The cell of claim 21, wherein the cell is a mammalian cell, optionally wherein the mammalian cell is a human cell.

23. (canceled)

24. The cell of claim 21, wherein the Cas9 is a catalytically inactive dCas9.

25. The cell of claim 21, wherein the Cas9 is fused to a DNA modifying protein domain.

26. A method comprising maintaining the cell of claim 21 under conditions that result in recording of molecular stimuli in the form of DNA mutations in the cell.

27. A method comprising delivering the cell of claim 21 to a subject, optionally wherein the subject is a human subject, and optionally wherein the subject has an inflammatory condition.

28-29. (canceled)
